(19) United States
(12) Patent Application Publication
(10) Pub. No.: US 2021/0353612 A1
(43) Pub. Date: Nov. 18, 2021

(54) HYPOXIA-INDUCIBLE FACTOR PROLYL HYDROXYLASE INHIBITORS FOR TREATING AGING-RELATED CONDITIONS

(71) Applicant: BioAge Labs, Inc., Richmond, CA (US)

(72) Inventors: Eric Kim MORGEN, Toronto (CA); Kristen Patricia FORTNEY, Crockett, CA (US); Peng LEONG, San Francisco, CA (US); Andrew Jarai HO, Berkeley, CA (US); Justin REBO, Richmond, CA (US); Yong PAN, Albany, CA (US)

(21) Appl. No.: 17/242,612

(22) Filed: Apr. 28, 2021

Related U.S. Application Data
(60) Provisional application No. 63/153,356, filed on Feb. 24, 2021.
(60) Provisional application No. 63/136,138, filed on Jan. 11, 2021.
(60) Provisional application No. 63/127,767, filed on Dec. 18, 2020.
(60) Provisional application No. 63/062,259, filed on Aug. 6, 2020.
(60) Provisional application No. 63/017,578, filed on Apr. 29, 2020.

(51) Int. Cl. A61K 31/4545; A61P 39/00

(52) U.S. Cl. CPC A61K 31/4545; A61P 39/00

(57) ABSTRACT

The present disclosure provides a method of treating a condition associated with aging, including a disease or condition selected from anemia, including unexplained anemia of the elderly, spontaneous anemia of aging, and anemia of inflammation in the elderly, sarcopenia, frailty, tissue injury, muscle injury, and ischemic damage, the method comprising administering to an elderly subject a therapeutically effective amount of a hypoxia-inducible factor prolyl hydroxylase inhibitor.

### 1. CROSS-REFERENCE TO RELATED APPLICATIONS

This application claims priority to and the benefit of U.S. Provisional Application No. 63/153,356, filed Feb. 24, 2021; 63/136,138, filed Jan. 11, 2021; 63/127,767, filed Dec. 18, 2020; 63/062,259, filed Aug. 6, 2020; and 63/017,578, filed Apr. 29, 2020; the entire disclosures of which are hereby incorporated by reference in their entireties.

### 2. BACKGROUND

As people age, they accumulate physiologic and pathophysiologic changes; these accumulated age-related changes predispose to death from various external and internal stressors. Indices of “frailty” have been developed as composite measures of such age-related changes. As the median age of the population increases, there is an increasing need for drugs that reduce or counteract the accumulation of age-related deficits and that therefore reduce measures of frailty in elderly individuals.

### 3. SUMMARY

We applied bioinformatic and machine learning approaches to analyze human data using survival predictor models and discovered an association of baseline HIF1α pathway protein levels with future aging outcomes. In particular, we discovered that higher circulating levels of HIF1α are associated with decreased all-cause mortality (p=0.0029)—that is, greater longevity—and that higher circulating levels of HIF-PH, which triggers degradation of HIF-1α, are associated with increased all-cause mortality (p=0.0201). In addition, the analysis demonstrated that higher levels of HIF1α are associated with better future physical function, whereas higher levels of HIF-PH are associated with worse future physical function.

We then discovered that HIF-1α serum protein concentration decreases with age in human healthy aging cohorts, and that expression of known downstream target genes of HIF-1α are affected, in turn, by the decrease in HIF-1α as humans age.

Based on these discoveries, we tested an inhibitor of HIF prolyl hydroxylase, BGE-117, for effects in aged mice. BGE-117 (also known as TP0463518 and TP518) has the structure shown below:

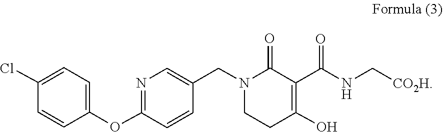 [SMILES: O=C(O)CNC(=O)CC1=C(O)CCN(Cc2ccc(Oc3ccc(Cl)cc3)nc2)C1=O]

In a first set of experiments, we demonstrated that old mice (27 months old) treated with BGE-117 exhibited a statistically significant (p<0.001) increase in voluntary activity as compared to age-matched controls, indicating a reduction in frailty and improved physical health.

In addition, we discovered that 27-month old mice treated with BGE-117 showed increased hemoglobin levels. Although BGE-117 was known to inhibit HIF-PH and increase erythropoietin (EPO) production in normal healthy human volunteers (Shinfuku et al., Am. J. Nephrol. 48(3):157-164 (2018)) and patients with chronic kidney disease, and has been shown to increase hemoglobin levels in 5/6 nephrectomized young rats (Kato et al., J. Pharmacol. Exp. Ther. 371:675-683 (2019), its effects on aged individuals with normal kidney function had not previously been reported.

We then discovered that aged mice (23-month old and 27-month old) spontaneously develop anemia of aging, and that this anemia is accompanied by elevated levels of inflammatory cytokines IL-6 and TNFα. This discovery suggested that an underlying etiology of the spontaneous anemia is anemia of inflammation. We then sought to determine whether the beneficial effects of BGE-117 would still be observed in old mice with anemia, and in particular, anemia of inflammation, or instead is limited to old mice with normal baseline hemoglobin levels and without elevated levels of inflammatory cytokines. Selecting mice in each age cohort that had high levels of inflammatory cytokines, we demonstrated that even in old mice with anemia of inflammation, BGE-117 is effective in increasing hemoglobin level.

Next, we demonstrated that another HIF-PH inhibitor, roxadustat, is also capable of increasing hemoglobin in aged animals exhibiting spontaneous inflammation and anemia of inflammation, thus demonstrating that HIF-PH inhibitors as a class are effective in treating anemia in old animals, including old animals with anemia of inflammation.

Accordingly, in a first aspect, the present disclosure provides methods of treating an aging-related morbidity, comprising: administering a therapeutically effective amount of a hypoxia-inducible factor prolyl hydroxylase (HIF-PH) inhibitor to a human subject older than 40 years old who has, or is at risk for developing, an aging-related morbidity.

In some embodiments, the aging-related morbidity is anemia of aging.

In some embodiments, the anemia of aging is anemia of inflammation in the elderly (AI).

In some embodiments, the age-related morbidity is anemia induced by acute medical events, processes, or hospital admissions.

In some embodiments, the human subject has a CRP level of greater than 2 mg/L. In some embodiments, the human subject has a CRP level of greater than 4 mg/L. In some embodiments, the human subject has a CRP level of greater than 6 mg/L. In some embodiments, the human subject has a CRP level of greater than 8 mg/L. In some embodiments, the human subject has a CRP level of greater than 10 mg/L. In some embodiments, the anemia is an unexplained anemia in the elderly (UAE).

In some embodiments, the human subject has a CRP level of less than 10 mg/L. In some embodiments, the human subject has a CRP level of less than 8 mg/L. In some embodiments, the human subject has a CRP level of less than 6 mg/L. In some embodiments, the human subject has a CRP level of less than 4 mg/L. In some embodiments, the human subject has a CRP level of less than 2 mg/L.

In some embodiments, the human subject has elevated pre-treatment levels of IL-6. In some embodiments, the human subject has a pre-treatment IL-6 level of greater than 2.5 pg/ml. In some embodiments, the human subject has a pre-treatment IL-6 level of greater than 5 pg/ml. In some embodiments, the human subject has a pre-treatment IL-6 level of greater than 10 pg/ml.

In some embodiments, the human subject has a pre-treatment TNFα level of greater than 7 pg/ml. In some embodiments, the human subject has a pre-treatment TNFα level of greater than 8 pg/ml. In some embodiments, the human subject has a pre-treatment TNFα level of greater than 9 pg/ml.

In some embodiments, the human subject has a pre-treatment TNFα level of greater than 10 pg/ml. In some embodiments, the human subject has a pre-treatment hemoglobin level below 12 g/dL. In some embodiments, the human subject has a pre-treatment hemoglobin level below 10 g/dL.

In some embodiments, the human subject has a pre-treatment hemoglobin level of less than 13 g/dL (male) or 12 g/dL (female). In some embodiments, the human subject has a pre-treatment hemoglobin level of less than 11 g/dL (male) or 10 g/dL (female). In some embodiments, the human subject has a pre-treatment hemoglobin level of less than 9 g/dL (male) or 8 g/dL (female).

In some embodiments, the human subject has an eGFR of greater than 30 ml/min.

In some embodiments, the human subject has an eGFR of greater than 50 ml/min.

In some embodiments, the human subject has normal B12 and folate levels.

In some embodiments, the human subject has serum ferritin (SF) greater than 100 and/or tumor inflammation signature (TIS) greater than 20%.

In some embodiments, the human subject does not have a renal disease.

In some embodiments, the human subject does not have a chronic kidney disease (CKD).

In some embodiments, the human subject does not have chronic kidney disease (CKD) stage 3-5.

In some embodiments, the human subject is not on hemodialysis.

In some embodiments, the human subject does not have anemia.

In some embodiments, the age-related morbidity is frailty.

In some embodiments, the age-related morbidity is fatigue.

In some embodiments, the human subject has reduced muscle strength. In some embodiments, the human subject has reduced capillary density.

In some embodiments, the human subject has reduced muscle force.

In some embodiments, the human subject has a reduction in lower limb muscle mass.

In some embodiments, the human subject has a reduction in upper limb muscle mass.

In some embodiments, the human subject has a reduction in muscle volume. In some embodiments, the muscle volume is the muscle volume of one or more upper limb muscles selected from the group consisting of: shoulder abductors, shoulder adductors, elbow flexors, elbow extensors, wrist flexors, and wrist extensors.

In some embodiments, the human subject has not been diagnosed with any disease except age-related frailty.

In some embodiments, the human subject has sarcopenia.

In some embodiments, the human subject has a reduction in capillary density.

In some embodiments, the human subject is older than 50.

In some embodiments, the human subject is older than 55.

In some embodiments, the human subject is older than 60.

In some embodiments, the human subject is older than 65.

In some embodiments, the human subject is older than 70.

In some embodiments, the human subject is older than 75.

In some embodiments, the human subject is older than 80.

In some embodiments, the human subject is older than 85.

In some embodiments, the HIF-PH inhibitor is a compound represented by the following general formula (I′):

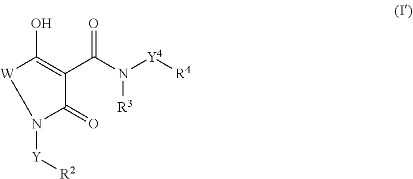

wherein in formula (I′), W represents the formula —CR¹¹R¹²CR¹³R¹⁴;
R¹¹ represents a hydrogen atom, C₁₋₄ alkyl, or phenyl;
R¹² represents a hydrogen atom, a fluoride atom or C₁₋₄ alkyl; or
R¹¹ and R¹², together with the adjacent carbon atom, form a C₃₋₈ cycloalkane or a 4- to 8-membered saturated heterocycle containing an oxygen atom;
R¹³ represents a hydrogen atom, carbamoyl, C₁₋₄ alkyl, wherein the C₁₋₄ alkyl is optionally substituted by one group selected from the group consisting of hydroxy, C₁₋₃ alkoxy, and di-C₁₋₃ alkylamino, halo-C₁₋₄ alkyl, phenyl, pyridyl, benzyl, or phenethyl;
R¹⁴ represents a hydrogen atom, C₁₋₄ alkyl, or halo-C₁₋₄ alkyl; or
R¹³ and R¹⁴, together with the adjacent carbon atom, form a C₃₋₈ cycloalkane, a 4- to 8-membered saturated heterocycle containing an oxygen atom, or a 4- to 8-membered saturated heterocycle containing a nitrogen atom, wherein the 4- to 8-membered saturated heterocycle containing a nitrogen atom is optionally substituted by one or two groups which are the same or different and are selected from the group consisting of methyl, benzyl, phenylcarbonyl, and oxo; or
R¹² and R¹³, together with the adjacent carbon atoms, form a C₃₋₈ cycloalkane;
Y represents a single bond or C₁₋₆ alkanediyl, wherein the C₁₋₆ alkanediyl is optonally substituted by one hydroxyl, and one of the carbon atoms in the C₁₋₆ alkanediyl is optionally substituted by C₃₋₆ cycloalkanel,1-diyl;
R² represents:
a hydrogen atom,
C₁₋₆ akyl,
C₃₋₈ cycloalkyl, wherein the C₃₋₈ cycloalkyl is optionally substituted by one or two groups which are the same or different and are selected from the group consisting of C₁₋₆ alkyl which is optionally substituted by one phenyl, phenyl which is optionally substituted by one group selected from the group consisting of a halogen atom and halo-C₁₋₆ alkyl, C₁₋₆ alkoxy which is optionally substituted by one group selected from the group consisting of C₃₋₈ cycloalkyl, phenyl optionally substituted by one group selected from the group consisting of a halogen atom and C₁₋₆ alkyl, and pyridyl optionally substituted by one halogen atom, C₃₋₈ cycloalkoxy, phenoxy which is optionally substituted by one group selected from the group consisting of a halogen atom, C₁₋₆ alkyl, C₃₋₈ cycloalkyl, and halo-C₁₋₆ alkyl, and pyridyloxy which is optionally substituted by one group selected from the group consisting of a halogen atom, C₁₋₆ alkyl, C₃₋₈ cycloalkyl, and halo-C₁₋₆ alkyl,
phenyl, wherein the phenyl is optionally substituted by one to three groups which are the same or different and are selected from group α3 of substituents,
naphthyl,
indanyl,
tetrahydronaphthyl,
pyrazolyl,
imidazolyl,
isoxazolyl,
oxazolyl, wherein the pyrazolyl, imidazolyl, isoxazolyl, and oxazolyl are optionally substituted by one or two groups which are the same or different and are selected from the group consisting of C₁₋₆ alkyl and phenyl which is optionally substituted by one group selected from the group consisting of a halogen atom and C₁₋₆ alkyl,
thiazoyl, wherein the thiazoyl is optionally substituted by one or two groups which are the same or different and are selected from the group consisting of C₁₋₆ alkyl, phenyl which is optionally substituted by one group selected from the group consisting of a halogen atom and C₁₋₆ alkyl, and morpholino,
pyridyl, wherein the pyridyl is optionally substituted by one or two groups which are the same or different and are selected from group α5 of substituents,
pyridazinyl,
pyrimidinyl,
pyrazinyl,
wherein the pyridazinyl, pyrimidinyl, and pyrazinyl are optionally substituted by one group selected from the group consisting of C₁₋₆ alkyl, halo-C₁₋₆ alkyl, C₃₋₈ cycloalkyl, phenyl, C₁₋₆ alkoxy which is optionally substituted by one C₃₋₈ cycloalkyl, and phenoxy which is optionally substituted by one group selected from the group consisting of a halogen atom, C₁₋₆ alkyl, and C₃₋₈ cycloalkyl,
benzothiophenyl,
quinolyl,
methylenedioxyphenyl, wherein the methylenedioxyphenyl is optionally substituted by one or two fluorine atoms,
4- to 8-membered saturated heterocyclyl containing a nitrogen atom, wherein the 4- to 8-membered saturated heterocyclyl containing a nitrogen atom is optionally substituted by one group selected from the group consisting of pyrimidinyl, phenyl-C₁₋₃ alkyl, C₃₋₈ cycloalkyl-C₁₋₃ alkylcarbonyl, and phenyl-C₁₋₃ alkoxycarbonyl, or

the following formula (I″)
—CONR⁵CH₂—R⁶ (I″), wherein in formula (I″):
R⁵ represents a hydrogen atom or C₁₋₃ alkyl, and R⁶ represents phenyl which is optionally substituted by one group selected from the group consisting of a halogen atom, C₁₋₆ alkyl, halo-C₁₋₆ alkyl, and phenyl,
group α3 of substituents consists of:
hydroxy,
cyano,
carboxy,
a halogen atom,
C₁₋₆ alkyl, wherein the C₁₋₆ alkyl is optionally substituted by one group selected from the group consisting of C₃₋₈ cycloalkyl, phenyl, C₁₋₆ alkoxy which is optionally substituted by one C₃₋₈ cycloalkyl optionally substituted by one C₁₋₆ alkyl , phenoxy which is optionally substituted by one C₁₋₆ alkyl, and pyridyloxy which is optionally substituted by one group selected from the group consisting of C₁₋₆ alkyl and halo-C₁₋₆ alkyl,
halo-C₁₋₆ alkyl,
C₃₋₈ cycloalkyl, wherein the C₃₋₈ cycloalkyl is optionally substituted by one or two halogen atoms,
C₃₋₈ cycloalkenyl, wherein the C₃₋₈ cycloalkenyl is optionally substituted by one or two halogen atoms,
phenyl, wherein the phenyl is optionally substituted by one to three groups which are the same or different and are selected from group α4 of substituents,
thienyl, wherein the thienyl is optionally substituted by one C₁₋₆ alkyl,
pyrazolyl, wherein the pyrazolyl is optionally substituted by one C₁₋₆ alkyl,
isoxazolyl,
thiazoyl, wherein the thiazoyl is optionally substituted by one or two groups which are the same or different and are selected from the group consisting of hydroxy, C₁₋₆ alkyl, and C₁₋₆ alkoxy,
pyridyl, wherein the pyridyl is optionally substituted by one group selected from the group consisting of carboxy, hydroxy, amino, a halogen atom, C₁₋₆ alkyl, halo-C₁₋₆ alkyl, C₃₋₈ cycloalkyl, C₁₋₆ alkoxy, halo-C₁₋₆ alkoxy, and C₁₋₆ alkylsulfonyl,
pyrimidinyl, wherein the pyrimidinyl is optionally substituted by one amino,
quinolyl,
C₁₋₆ alkoxy, wherein the C₁₋₆ alkoxy is optionally substituted by one group selected from the group consisting of carboxy, hydroxy, carbamoyl, C₃₋₈ cycloalkyl which is optionally substituted by one C₁₋₆ alkyl, phenyl which is optionally substituted by one group selected from the group consisting of hydroxy, a halogen atom, C₁₋₆ alkyl, halo-C₁₋₆ alkyl, C₁₋₆ alkoxy, halo-C₁₋₆ alkoxy, and di-C₁₋₆ alkylamino, pyridyl which is optionally substituted by one group selected from the group consisting of a halogen atom and C₁₋₆ alkyl, benzotriazolyl, imidazothiazoyl, di-C₁₋₆ alkylamino, oxazolyl which is optionally substituted by one or two C₁₋₆ alkyls, pyrazolyl, which is optionally substituted by one or two C₁₋₆ alkyls, thiazoyl which is optionally substituted by one C₁₋₆ alkyl, and indazolyl which is optionally substituted by one C₁₋₆ alkyl,
halo-C₁₋₆ alkoxy,
C₂₋₆ alkenyloxy,
C₃₋₈ cycloalkoxy,
phenoxy, wherein the phenoxy is optionally substituted by one or two groups which are the same or different and are selected from the group consisting of a halogen atom, C₁₋₆ alkyl, halo-C₁₋₆ alkyl, C₁₋₆ alkoxy, and halo-C₁₋₆ alkoxy,
pyridyloxy, wherein the pyridyloxy is optionally substituted by one group selected from the group consisting of a halogen atom, C₁₋₆ alkyl, halo-C₁₋₆ alkyl, and C₃₋₈ cycloalkyl,
pyrimidinyloxy,
piperazinyl, wherein the piperazinyl is optionally substituted by one C₁₋₆ alkyl,
mono-C₁₋₆ alkylaminocarbonyl, wherein the C₁₋₆ alkyl in the mono-C₁₋₆ alkylaminocarbonyl is optionally substituted by one group selected from the group consisting of carboxy, hydroxy, di-C₁₋₆ alkylamino, pyridyl, phenyl, and 2-oxopyrrolidinyl,
di-C₁₋₆ alkylaminocarbonyl, wherein the two C₁₋₆ alkyls in the di-C₁₋₆ alkylaminocarbonyl, together with the adjacent nitrogen atom, optionally form a 4- to 8-membered saturated heterocycle containing a nitrogen atom,
C₁₋₆ alkylsulfanyl, and
C₁₋₆ alkylsulfonyl;
   group α4 of substituents consists of: carboxy,
   cyano,
   hydroxy,
   sulfamoyl,
   a halogen atom,
   C₁₋₆ alkyl,
   halo-C₁₋₆ alkyl,
   C₃₋₈ cycloalkyl,
   phenyl,
   C_{1.6} alkoxy,
   halo-C₁₋₆ alkoxy,
   C₁₋₆ alkylcarbonyl,
   di-C₁₋₆ alkylaminocarbonyl,
   C₁₋₆ alkylsulfonyl,
   mono-C₁₋₆ alkylaminosulfonyl, wherein the C₁₋₆ alkyl in the mono-C₁₋₆ alkylaminosulfonyl is optionally substituted by one hydroxy, and
di-C₁₋₆ alkylaminosulfonyl;

group α5 of substituents consist of:
a halogen atom,
C₁₋₆ alkyl,
halo-C₁₋₆ alkyl,
C₁₋₆ alkoxy, wherein the C₁₋₆ alkoxy is optionally substituted by one group selected from the group consisting of C₃₋₈ cycloalkyl which is optionally substituted by one C₁₋₆ alkyl and phenyl which is optionally substituted by one group selected from the group consisting of a halogen atom and C₁₋₆ alkyl,
halo-C₁₋₆ alkoxy,
phenyl, wherein the phenyl is optionally substituted by one group selected from group α6 of substituents,
pyridyl,
phenoxy, wherein the phenoxy is optionally substituted by one or two groups which are the same or different and are selected from the group consisting of a halogen atom, cyano, C₁₋₆ alkyl, halo-C₁₋₆ alkyl, C₃₋₈ cycloalkyl, halo-C₁₋₆ alkoxy and C₁₋₆ alkoxy which is optionally substituted by one phenyl, and,
pyridyloxy, wherein the pyridyloxy is optionally substituted by one C₁₋₆ alkyl, and
phenylsulfanyl, wherein the phenylsulfanyl is optionally substituted by one halogen atom;

group α6 of substituents consists of:
a halogen atom,
C₁₋₆ alkyl,
halo-C₁₋₆ alkyl,
C₃₋₈ cycloalkyl,
C₁₋₆ alkoxy, and
halo-C₁₋₆ alkoxy;
Y⁴ represents C₁₋₄ alkanediyl;
R³ represents a hydrogen atom or methyl;
R⁴ represents —COOH, —CONHOH, or tetrazolyl;
or a pharmaceutically acceptable salt thereof.

In some embodiments, in the aforementioned general formula (I′):
Y⁴ is methanediyl,
R³ is a hydrogen atom,
R⁴ is —COOH, or a pharmaceutically acceptable salt thereof.

In some embodiments, in the aforementioned general formula (I′), the compound is represented by general formula (I′-2):

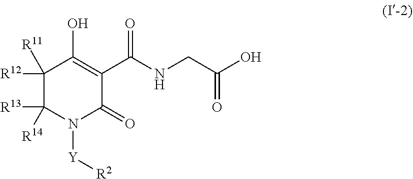

wherein in formula (I′-2):
   R¹¹ is a hydrogen atom, a fluorine atom, C₁₋₄ alkyl, or phenyl,
   R¹² is a hydrogen atom, a fluorine atom, or C₁₋₄ alkyl, or
   R¹¹ and R¹², together with the adjacent carbon atom, form a C₃₋₈ cycloalkane or a 4- to 8-membered saturated heterocycle containing an oxygen atom;
   R¹³ is a hydrogen atom, carbamoyl, C₁₋₄ alkyl, wherein the C₁₋₄ alkyl is optionally substituted by one group selected from the group consisting of hydroxy, C₁₋₃ alkoxy, and di-C₁₋₃ alkylamino, halo-C₁₋₄ alkyl, phenyl, pyridyl, benzyl, or phenethyl;
   R¹⁴ is a hydrogen atom, C₁₋₄ alkyl, or halo-C₁₋₄ alkyl, or
   R¹³ and R¹⁴, together with the adjacent carbon atom, form a C₃₋₈ cycloalkane, a 4- to 8-membered saturated heterocycle containing an oxygen atom, or a 4- to 8-membered saturated heterocycle containing a nitrogen atom, wherein the 4- to 8-membered saturated heterocycle containing a nitrogen atom is optionally substituted by one or two groups which are the same or different and are selected from the group consisting of methyl, benzyl, phenylcarbonyl, and oxo, or
   R¹² and R¹³, together with the adjacent carbon atoms, form a C₃₋₈ cycloalkane,
   or a pharmaceutically acceptable salt thereof.

In some embodiments, in the aforementioned general formula (I′-2):
Y is a single bond or C₁₋₆ alkanediyl, wherein one of the carbon atoms in the C₁₋₆ alkanediyl is optionally substituted by C₃₋₆ cycloalkane-1,1-diyl,
R² is:
   C₃₋₈ cycloalkyl, wherein the C₃₋₈ cycloalkyl is optionally substituted by one or two groups which are the same or different and are selected from the group consisting of C₁₋₆ alkyl which is optionally substituted by one phenyl, phenyl which is optionally substituted by one halo-C₁₋₆ alkyl, C₁₋₆ alkoxy which is optionally substituted by one group selected from the group consisting of C₃₋₈ cycloalkyl, phenyl optionally substituted by one group selected from the group consisting of a halogen atom and C₁₋₆ alkyl, and pyridyl optionally substituted by one halogen atom, C₃₋₈ cycloalkoxy, phenoxy which is optionally substituted by one group selected from the group consisting of a halogen atom, C₁₋₆ alkyl, C₃₋₈ cycloalkyl, and halo-C₁₋₆ alkyl, and pyridyloxy which is optionally substituted by one group selected from the group consisting of a halogen atom, C₁₋₆ alkyl, C₃₋₈ cycloalkyl, and halo-C₁₋₆ alkyl,
phenyl, wherein the phenyl is optionally substituted by one to three groups which are the same or different and are selected from the aforementioned group α3 of substituents,
   naphthyl,
   indanyl,
   tetrahydronaphthyl,
   pyrazolyl, wherein the pyrazolyl is optionally substituted by one or two groups which are the same or different and are selected from the group consisting of C₁₋₆ alkyl and phenyl which is optionally substituted by one C₁₋₆ alkyl,
   imidazolyl, wherein the imidazolyl is optionally substituted by one group selected from the group consisting of C₁₋₆ alkyl and phenyl,
   isoxazolyl, wherein the isoxazolyl is optionally substituted by one phenyl which is optionally substituted by one halogen atom,
   oxazolyl, wherein the oxazolyl is optionally substituted by one or two groups which are the same or different and are selected from the group consisting of C₁₋₆ alkyl and phenyl,
   thiazoyl, wherein the thiazoyl is optionally substituted by one group selected from the group consisting of C₁₋₆ alkyl, phenyl, and morpholino,
   pyridyl, wherein the pyridyl is optionally substituted by one or two groups which are the same or different and are selected from the aforementioned group α5 of substituents,
   pyridazinyl, wherein the pyridazinyl is optionally substituted by one C₁₋₆ alkoxy which is optionally substituted by one C₃₋₈ cycloalkyl,
   pyrimidinyl, wherein the pyrimidinyl is optionally substituted by one group selected from the group consisting of halo-C₁₋₆ alkyl, C₃₋₈ cycloalkyl, phenyl, and phenoxy which is optionally substituted by one C₁₋₆ alkyl,
   pyrazinyl, wherein the pyrazinyl is optionally substituted by one group selected from the group consisting of C₁₋₆ alkoxy which is optionally substituted by one C₃₋₈ cycloalkyl and phenoxy which is optionally substituted by one group selected from the group consisting of a halogen atom, C₁₋₆ alkyl, and C₃₋₈ cycloalkyl,
   benzothiophenyl,
   quinolyl, or
   methylenedioxyphenyl, wherein the methylenedioxyphenyl is optionally substituted by one or two fluorine atoms,
   or a pharmaceutically acceptable salt thereof.

In some embodiments, in the aforementioned general formula (I′-2):
R¹¹ is a hydrogen atom,
R¹² is a hydrogen atom,
R¹³ is a hydrogen atom,
R¹⁴ is a hydrogen atom,
Y is methanediyl,
R² is:
   phenyl, wherein the phenyl is substituted by one group selected from the group consisting of phenyl which is optionally substituted by one or two groups which are the same or different and are selected from the group consisting of carboxy, cyano, hydroxy, sulfamoyl, a halogen atom, C₁₋₆ alkyl, halo-C₁₋₆ alkyl, C₃₋₈ cycloalkyl, phenyl, C₁₋₆ alkoxy, halo-C₁₋₆ alkoxy, C₁₋₆ alkylcarbonyl, di-C₁₋₆ alkylaminocarbonyl, C₁₋₆ alkyl sulfonyl, di-C₁₋₆ alkylaminosulfonyl and mono-C₁₋₆ alkylaminosulfonyl, wherein the C₁₋₆ alkyl in the mono-C₁₋₆alkylaminosulfonyl is optionally substituted by one hydroxy, pyridyl which is optionally substituted by one group selected from the group consisting of carboxy, hydroxy, amino, a halogen atom, C₁₋₆ alkyl, halo-C₁₋₆ alkyl, C₃₋₈ cycloalkyl, C₁₋₆ alkoxy, and C₁₋₆ alkylsulfonyl, phenoxy which is optionally substituted by one or two groups which are the same or different and are selected from the group consisting of a halogen atom, C₁₋₆ alkyl, C₁₋₆ alkoxy, and halo-C₁₋₆ alkoxy, and pyridyloxy which is optionally substituted by one group selected from the group consisting of a halogen atom, C₁₋₆ alkyl, halo-C₁₋₆ alkyl, and C₃₋₈ cycloalkyl, and said substituted phenyl represented by R² may further be substituted by one halogen atom;
   pyridyl, wherein the pyridyl is substituted by one group selected from the group consisting of phenyl which is optionally substituted by one group selected from the group consisting of a halogen atom, C₁₋₆ alkyl, halo-C₁₋₆ alkyl, C₃₋₈ cycloalkyl, C₁₋₆ alkoxy, and halo-C₁₋₆ alkoxy, pyridyl, phenoxy which is optionally substituted by one or two groups which are the same or different and are selected from the group consisting of a halogen atom, cyano, C₁₋₆ alkyl, halo-C₁₋₆ alkyl, C₃₋₈ cycloalkyl, halo-C₁₋₆ alkoxy and C₁₋₆ alkoxy optionally substituted by one phenyl, and pyridyloxy which is optionally substituted by one C₁₋₆ alkyl, and said substituted pyridyl represented by R² may further be substituted by one group selected from the group consisting of a halogen atom and C₁₋₆ alkyl; or
   pyrazinyl which is substituted by one phenoxy wherein the phenoxy is optionally substituted by one group selected from the group consisting of a halogen atom, C₁₋₆ alkyl, and C₃₋₈ cycloalkyl,
or a pharmaceutically acceptable salt thereof.

In some embodiments, the compound is selected from:

N-{[4-hydroxy-2-oxo-1-(4-phenoxybenzyl)-1,2,5,6-tetrahydro-3-pyridinyl]carbonyl}glycine;

N-[(4-hydroxy-1-{1[6-(4-methylphenoxy)-3-pyridinyl]methyl}-2-oxo-1,2,5,6-tetrahydro-3-pyridinyl)carbonyl]glycine;

N-({4-hydroxy-2-oxo-1-[(6-phenoxy-3-pyridinyl)methyl]-1,2,5,6-tetrahydro-3-pyridinyl}carbonyl)glycine;

N-({1-[4-(4-fluorophenoxy)benzyl]-4-hydroxy-2-oxo-1,2,5,6-tetrahydro-3-pyridinyl}carbonyl)glycine;

N-({4-hydroxy-1-[4-(4-methylphenoxy)benzyl]-2-oxo-1,2,5,6-tetrahydro-3-pyridinyl}carbonyl)glycine;

N-[(1-{[6-(4-cyanophenoxy)-3-pyridinyl]methyl}-4-hydroxy-2-oxo-1,2,5,6-tetrahydro-3-pyridinyl)carbonyl]glycine;

N-({4-hydroxy-2-oxo-1-[4-(2-pyrimidinyloxy)benzyl]-1,2,5,6-tetrahydro-3-pyridinyl}carbonyl)glycine;

N-[(1-{[6-(4-fluorophenoxy)-3-pyridinyl]methyl}-4-hydroxy-2-oxo-1,2,5,6-tetrahydro-3-pyridinyl)carbonyl]glycine;

N-[(1{[-6-(4-chlorophenoxy)-3-pyridinyl]methyl}-4-hydroxy-2-oxo-1,2,5,6-tetrahydro-3-pyridinyl)carbonyl]glycine;

N-{[4-hydroxy-2-oxo-1-({6-[4-(trifluoromethyl)phenoxy]-3-pyridinyl}methyl)-1,2,5,6-tetrahydro-3-pyridinyl]carbonyl}glycine;

N-[(4-hydroxy-1-{[6-(3-methylphenoxy)-3-pyridinyl]methyl}-2-oxo-1,2,5,6-tetrahydro-3-pyridinyl)carbonyl]glycine;

N-[(1-{[6-(3-fluorophenoxy)-3-pyridinyl]methyl}-4-hydroxy-2-oxo-1,2,5,6-tetrahydro-3-pyridinyl)carbonyl]glycine;

N-({4-hydroxy-1-[4-(3-methylphenoxy)benzyl]-2-oxo-1,2,5,6-tetrahydro-3-pyridinyl}carbonyl)glycine;

N-({1-[4-(3-fluorophenoxy)benzyl]-4-hydroxy-2-oxo-1,2,5,6-tetrahydro-3-pyridinyl}carbonyl)glycine;

N-[1-{[5-(4-fluorophenoxy)-2-pyridinyl]methyl}-4-hydroxy-2-oxo-1,2,5,6-tetrahydro-3-pyridinyl)carbonyl]glycine;

N-[(4-hydroxy-1-{[5-(4-methylphenoxy)-2-pyridinyl]methyl}-2-oxo-1,2,5,6-tetrahydro-3-pyridinyl)carbonyl]glycine;

N-({1-[4-(4-chlorophenoxy)benzyl]-4-hydroxy-2-oxo-1,2,5,6-tetrahydro-3-pyridinyl}carbonyl)glycine;

N-[(4-hydroxy-1-{4-[(6-methyl-3-pyridinyl)oxy]benzyl}-2-oxo-1,2,5,6-tetrahydro-3-pyridinyl)carbonyl]glycine;

N-[(1-{[6-(2-fluorophenoxy)-3-pyridinyl]methyl}-4-hydroxy-2-oxo-1,2,5,6-tetrahydro-3-pyridinyl)carbonyl]glycine;

N-[(4-hydroxy-1-{[6-(2-methylphenoxy)-3-pyridinyl]methyl}-2-oxo-1,2,5,6-tetrahydro-3-pyridinyl)carbonyl]glycine;

N-({1-[4-(2-fluorophenoxy)benzyl]-4-hydroxy-2-oxo-1,2,5,6-tetrahydro-3-pyridinyl}carbonyl)glycine;

N-({4-hydroxy-1-[4-(2-methylphenoxy)benzyl]-2-oxo-1,2,5,6-tetrahydro-3-pyridinyl}carbonyl)glycine;

N-[(1-{[6-(3-chlorophenoxy)-3-pyridinyl]methyl}-4-hydroxy-2-oxo-1,2,5,6-tetrahydro-3-pyridinyl)carbonyl]glycine;

N-{[4-hydroxy-2-oxo-1-({6-[3-(trifluoromethyl)phenoxy]-3-pyridinyl}methyl)-1,2,5,6-tetrahydro-3-pyridinyl]carbonyl}glycine;

N-({4-hydroxy-1-[4-(3-methoxyphenoxy)benzyl]-2-oxo-1,2,5,6-tetrahydro-3-pyridinyl}carbonyl)glycine;

N-{[4-hydroxy-2-oxo-1-({6-[3-(trifluoromethoxy)phenoxy]-3-pyridinyl}methyl)-1,2,5,6-tetrahydro-3-pyridinyl]carbonyl}glycine;

N-[(1-{4-[(5-fluoro-2-pyridinyl)oxy]benzyl}-4-hydroxy-2-oxo-1,2,5,6-tetrahydro-3-pyridinyl)carbonyl]glycine;

N-[(1-{4-[(5-chloro-2-pyridinyl)oxy]benzyl}-4-hydroxy-2-oxo-1,2,5,6-tetrahydro-3-pyridinyl)carbonyl]glycine;

N-[1-{[(6-(4-cyclopropylphenoxy)-3-pyridinyl]methyl}-4-hydroxy-2-oxo-1,2,5,6-tetrahydro-3-pyridinyl)carbonyl]glycine;

N-[(4-hydroxy-1-{4-[(5-methyl-2-pyridinyl)oxy]benzyl}-2-oxo-1,2,5,6-tetrahydro-3-pyridinyl)carbonyl]glycine;

N-{[4-hydroxy-2-oxo-1-(4-{[5-(trifluoromethyl)-2-pyridinyl]oxy}benzyl)-1,2,5,6-tetrahydro-3-pyridinyl]carbonyl}glycine;

N-{[4-hydroxy-1-({5-methyl-6-[(6-methyl-3-pyridinyl)oxy]-3-pyridinyl}methyl)-2-oxo-1,2,5,6-tetrahydro-3-pyridinyl]carbonyl}glycine;

N-[(1-{[5-(4-chlorophenoxy)-2-pyridinyl]methyl}-4-hydroxy-2-oxo-1,2,5,6-tetrahydro-3-pyridinyl)carbonyl]glycine;

N-[(4-hydroxy-1-{[6-(3-methoxyphenoxy)-3-pyridinyl]methyl}-2-oxo-1,2,5,6-tetrahydro-3-pyridinyl)carbonyl]glycine;

N-[(1-{4-[(6-chloro-3-pyridinyl)oxy]benzyl}-4-hydroxy-2-oxo-1,2,5,6-tetrahydro-3-pyridinyl)carbonyl]glycine;

N-{[4-hydroxy-2-oxo-1-({5-[4-(trifluoromethyl)phenoxy]-2-pyridinyl}methyl)-1,2,5,6-tetrahydro-3-pyridinyl]carbonyl}glycine;

N-{[4-hydroxy-2-oxo-1-(4-{[6-(trifluoromethyl)-3-pyridinyl]oxy}benzyl)-1,2,5,6-tetrahydro-3-pyridinyl]carbonyl}glycine;

N-[(1-{[6-(3-chloro-4-methylphenoxy)-3-pyridinyl]methyl}-4-hydroxy-2-oxo-1,2,5,6-tetrahydro-3-pyridinyl)carbonyl]glycine;

N-[(1-{[6-(3-fluoro-4-methylphenoxy)-3-pyridinyl]methyl}-4-hydroxy-2-oxo-1,2,5,6-tetrahydro-3-pyridinyl)carbonyl]glycine;

N-[(1-{[6-(4-fluoro-3-methylphenoxy)-3-pyridinyl]methyl}-4-hydroxy-2-oxo-1,2,5,6-tetrahydro-3-pyridinyl)carbonyl]glycine;

N-[(1-{[6-(4-ethylphenoxy)-3-pyridinyl]methyl}-4-hydroxy-2-oxo-1,2,5,6-tetrahydro-3-pyridinyl)carbonyl]glycine;

N-[(4-hydroxy-2-oxo-1-{[6-(4-propylphenoxy)-3-pyridinyl]methyl}-1,2,5,6-tetrahydro-3pyridinyl)carbonyl]glycine;

N-[(4-hydroxy-1-{[6-(4-isopropylphenoxy)-3-pyridinyl]methyl}-2-oxo-1,2,5,6-tetrahydro-3-pyridinyl)carbonyl]glycine;

N-[(4-hydroxy-1-{[5-(4-methylphenoxy)-2-pyrazinyl]methyl}-2-oxo-1,2,5,6-tetrahydro-3-pyridinyl)carbonyl]glycine;

N-({1-[4-(3,4-dimethylphenoxy)benzyl]-4-hydroxy-2-oxo-1,2,5,6-tetrahydro-3-pyridinyl}carbonyl)glycine;

N-[(1-{[5-chloro-6-(4-methylphenoxy)-3-pyridinyl]methyl}-4-hydroxy-2-oxo-1,2,5,6-tetrahydro-3-pyridinyl)carbonyl]glycine;

N-[(1-{[5-fluoro-6-(4-methylphenoxy)-3-pyridinyl]methyl}-4-hydroxy-2-oxo-1,2,5,6-tetrahydro-3-pyridinyl)carbonyl]glycine;

N-[(1-{4-[(5-cyclopropyl-2-pyridinyl)oxy]benzyl}-4-hydroxy-2-oxo-1,2,5,6-tetrahydro-3-pyridinyl)carbonyl]glycine;

N-[(4-hydroxy-1-{[2-(4-methylphenoxy)-5-pyrimidinyl]methyl}-2-oxo-1,2,5,6-tetrahydro-3-pyridinyl)carbonyl]glycine; N-[(1-{[6-(4-chlorophenoxy)-5-methyl-3-pyridinyl]methyl}-4-hydroxy-2-oxo-1,2,5,6-tetrahydro-3-pyridinyl)carbonyl]glycine;

N-[(1-{[5-(4-chlorophenoxy)-2-pyrazinyl]methyl}-4-hydroxy-2-oxo-1,2,5,6-tetrahydro-3-pyridinyl)carbonyl]glycine; and

N-[(1-{[5-(4-cyclopropylphenoxy)-2-pyrazinyl]methyl}-4-hydroxy-2-oxo-1,2,5,6-tetrahydro-3-pyridinyl)carbonyl]glycine, or

a pharmaceutically acceptable salt thereof.

In some embodiments, the compound of formula (I′) is represented by general formula (I):

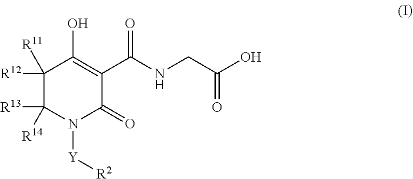

wherein in formula (I):
R¹¹ is a hydrogen atom, C₁₋₄ alkyl, or phenyl,
R¹² is a hydrogen atom or C₁₋₄ alkyl, or
R¹¹ and R¹², together with the adjacent carbon atom, form a C₃₋₈ cycloalkane or a 4- to 8-membered saturated heterocycle containing an oxygen atom;
R¹³ is a hydrogen atom, C₁₋₄ alkyl, halo-C₁₋₄ alkyl, phenyl, benzyl, or phenethyl,
R¹⁴ is a hydrogen atom or C₁₋₄ alkyl, or
R¹³and R¹⁴, together with the adjacent carbon atom, form a C₃₋₈ cycloalkane or a 4- to 8-membered saturated heterocycle containing an oxygen atom, or
R¹² and R¹³, together with the adjacent carbon atoms, form a C₃₋₈ cycloalkane;
Y is a single bond or C₁₋₆ alkanediyl, wherein one of the carbon atoms in the C₁₋₆ alkanediyl is optionally substituted by C₃₋₆ cycloalkane-1,1-diyl;
R² is:
   C₃₋₈ cycloalkyl, wherein the C₃₋₈ cycloalkyl is optionally substituted by one group selected from the group consisting of phenyl and benzyl,
   phenyl, wherein the phenyl is optionally substituted by one to three groups which are the same or different and are selected from group α1 of substituents,
      naphthyl,
      indanyl,
      tetrahydronaphthyl,
      pyrazolyl, wherein the pyrazolyl is substituted by one phenyl, which is optionally substituted by one C₁₋₆ alkyl and may further be substituted by one C₁₋₆ alkyl,
      imidazolyl, wherein the imidazolyl is substituted by one phenyl,
      isoxazolyl, wherein the isoxazolyl is substituted by one phenyl which is optionally substituted by one halogen atom,
      oxazolyl, wherein the oxazolyl is substituted by one phenyl and may further be substituted by one C₁₋₆ alkyl,
      thiazoyl, wherein the thiazoyl is substituted by one phenyl,
      pyridyl, wherein the pyridyl is substituted by one group selected from the group consisting of phenyl, phenoxy which is optionally substituted by one group selected from the group consisting of a halogen atom, cyano, C₁₋₆ alkyl, halo-C₁₋₆ alkyl, C₃₋₈ cycloalkyl, C₁₋₆ alkoxy, and halo-C₁₋₆ alkoxy), and phenylsulfanyl which is optionally substituted by one halogen atom,
      pyrimidinyl, wherein the pyrimidinyl is substituted by one group selected from the group consisting of cyclohexyl and phenyl,
      benzothiophenyl,
      quinolyl, or
      methylenedioxyphenyl, wherein the methylenedioxyphenyl is optionally
      substituted by one or two fluorine atoms;
group α1 of substituents consists of:
   a halogen atom,
   C₁₋₆ alkyl, wherein the C₁₋₆ alkyl is optionally substituted by one group selected from the group consisting of C₃₋₈ cycloalkyl, phenyl, and C₁₋₆ alkoxy which is optionally substituted by one C₃₋₈ cycloalkyl optionally substituted by one C₁₋₆ alkyl,
   halo-C₁₋₆ alkyl,
   C₃₋₈ cycloalkyl,
   phenyl, wherein the phenyl is optionally substituted by one to three groups which are the same or different and are selected from group α2 of substituents,
   thienyl,
   pyrazolyl, wherein the pyrazolyl is optionally substituted by one C₁₋₆ alkyl, isoxazolyl,
   thiazoyl, wherein the thiazoyl is optionally substituted by one or two C₁₋₆ alkyls,
   pyridyl, wherein the pyridyl is optionally substituted by one group selected from the group consisting of C₁₋₆ alkyl, halo-C₁₋₆ alkyl, C₁₋₆ alkoxy, and halo-C₁₋₆ alkoxy,
   quinolyl,
   C₁₋₆ alkoxy, wherein the C₁₋₆ alkoxy is optionally substituted by one group selected from the group consisting of C₃₋₈ cycloalkyl and phenyl which is optionally substituted by one group selected from the group consisting of a halogen atom and C₁₋₆ alkyl,
   halo-C₁₋₆ alkoxy,
   C₂₋₆ alkenyloxy,
   C₃₋₈ cycloalkoxy,
   phenoxy, wherein the phenoxy is optionally substituted by one group selected from the group consisting of a halogen atom, C₁₋₆ alkyl, halo-C₁₋₆ alkyl, C₁₋₆ alkoxy, and halo-C₁₋₆ alkoxy,
   pyridyloxy, wherein the pyridyloxy is optionally substituted by one group selected from the group consisting of a halogen atom, C₁₋₆ alkyl, and halo-C₁₋₆ alkyl, and
   C₁₋₆alkylsulfanyl;
group α2 of substituents consists of a halogen atom, cyano, hydroxy, C₁₋₆ alkyl, halo-C₁₋₆ alkyl, phenyl, C₁₋₆ alkoxy, halo-C₁₋₆ alkoxy, C₁₋₆ alkylcarbonyl, and di-C₁₋₆ alkylaminosulfonyl, or a pharmaceutically acceptable salt thereof.

In some embodiments, the compound is N-[(1{[6-(4-chlorophenoxy)-3-pyridinyl]methyl}-4-hydroxy-2-oxo-1,2,5,6-tetrahydro-3-pyridinyl)carbonyl]glycine, or a pharmaceutically acceptable salt thereof.

In some embodiments, the compound is 2-[[1-[[6-(4-chlorophenoxy)pyridin-3-yl]methyl]-4-hydroxy-6-oxo-2,3-dihydropyridine-5-carbonyl]amino]acetic acid.

In some embodiments, the compound is N-[(1-{[6-(4-cyclopropylphenoxy)-3-pyridinyl]methyl}-4-hydroxy-2-oxo-1,2,5,6-tetrahydro-3-pyridinyl)carbonyl]glycine, or a pharmaceutically acceptable salt thereof.

In some embodiments, the compound is N-[(4-hydroxy-1-{[6-(3-methylphenoxy)-3-pyridinyl]methyl}-2-oxo-1,2,5,6-tetrahydro-3-pyridinyl)carbonyl]glycine, or a pharmaceutically acceptable salt thereof.

In some embodiments, the compound is N-[(1-{[6-(3-fluorophenoxy)-3-pyridinyl]methyl}-4-hydroxy-2-oxo-1,2,5,6-tetrahydro-3-pyridinyl)carbonyl]glycine, or a pharmaceutically acceptable salt thereof.

In some embodiments, the compound is N-[(4-hydroxy-1-{4-[(6-methyl-3-pyridinyl)oxy]benzyl}-2-oxo-1,2,5,6-tetrahydro-3-pyridinyl)carbonyl]glycine, or a pharmaceutically acceptable salt thereof.

In some embodiments, the HIF-PH inhibitor is Desidustat, or a pharmaceutically acceptable salt thereof.

In some embodiments, the compound is the compound of Formula (3):

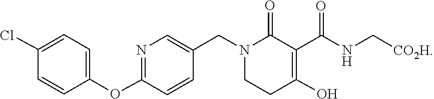 [SMILES: O=C(O)CNC(=O)CC1=C(O)CCN(Cc2ccc(Oc3ccc(Cl)cc3)nc2)C1=O]

In some embodiments, the HIF-PH inhibitor is Enarodustat, or a pharmaceutically acceptable salt thereof.

In some embodiments, the HIF-PH inhibitor is Molidustat, or a pharmaceutically acceptable salt thereof.

In some embodiments, the HIF-PH inhibitor is Roxadustat, or a pharmaceutically acceptable salt thereof.

In some embodiments, the HIF-PH inhibitor is Daprodustat, or a pharmaceutically acceptable salt thereof.

In some embodiments, the HIF-PH inhibitor is Vadadustat, or a pharmaceutically acceptable salt thereof.

In some embodiments, the HIF-PH inhibitor is 1-(6-(2,6-dimethylphenoxy)-7-fluoro-4-oxo-3,4-dihydroquinazolin-2-yl)-1H-pyrazole-4-carboxylic acid (JNJ-42905343) (JNJ-42905343).

In some embodiments, the HIF-PH inhibitor is JNJ-42041935.

In some embodiments, the condition is frailty.

In some embodiments, the condition is age associated frailty.

In some embodiments, the dose of HIF-PH inhibitor is at least 0.5 mg/kg. In some embodiments, the dose of HIF-PH inhibitor is at least 2 mg/kg. In some embodiments, the dose of HIF-PH inhibitor is at least 4 mg/kg. In some embodiments, the dose of HIF-PH inhibitor is at least 8 mg/kg. In some embodiments, the dose of HIF-PH inhibitor is at least 12 mg/kg. In some embodiments, the dose of HIF-PH inhibitor is at least 14 mg/kg. In some embodiments, the dose of HIF-PH inhibitor is at least 16 mg/kg.

In some embodiments, the dose is 0.5 mg/kg. In some embodiments, the dose is 1 mg/kg. In some embodiments, the dose is 2 mg/kg. In some embodiments, the dose is 2.5 mg/kg to 160 mg/kg.

In some embodiments, the dose of HIF-PH inhibitor is at least 0.01 mg/kg PO per day.

In some embodiments, the dose of HIF-PH inhibitor is at least 0.1 mg/kg PO per day.

In some embodiments, the dose of HIF-PH inhibitor is at least 0.05 mg/kg PO per day.

In some embodiments, the dose of HIF-PH inhibitor is at least 2 mg/kg PO per day.

In some embodiments, the dose of HIF-PH inhibitor is at least 3 mg/kg PO per day.

In some embodiments, the dose of HIF-PH inhibitor is at least 5 mg/kg PO per day.

In some embodiments, the dose of HIF-PH inhibitor is at least 10 mg/kg PO per day.

In some embodiments, the dose is 1 to 30 mg.

In some embodiments, the dose is 1 to 11 mg.

In some embodiments, the dose is 12 to 30 mg.

In some embodiments, the HIF-PH inhibitor is administered orally.

In some embodiments, the dose is administered daily.

In some embodiments, the dose is administered as a plurality of equally or unequally divided sub-doses.

### 4. BRIEF DESCRIPTION OF THE SEVERAL VIEWS OF THE DRAWINGS

These and other features, aspects, and advantages of the present disclosure will become better understood with regard to the following description, and accompanying drawings, where:

FIG. 1 shows the structure of BGE-117, referred to interchangeably herein as TP0463518.

FIGS. 2A-2B graph results from a bioinformatic survival model examining the relationship between serum levels of a given protein and future risk of all-cause mortality (i.e. longevity) in human healthy aging cohorts, with non-public clinical outcome data and proteomics data generated on archived samples. FIG. 2A shows a Kaplan-Meier curve of survival probability for humans in the top 20% (solid line) versus bottom 20% (dashed line) of HIF-1α protein levels, demonstrating that, in humans, higher circulating levels of HIF1 alpha are associated with decreased all-cause mortality (p=0.0029). FIG. 2B shows a similar model, with restricted cubic splines to generate a non-linear fit of survival hazard ratio by protein levels, where higher circulating levels of hypoxia-inducible factor-prolyl hydroxylase (HIF-PH) are associated with increased all-cause mortality (p=0.0201). The dashed lines show the 95% confidence interval for the solid line. The hazard ratio for HIFα (0.90) and HIF-PH (1.08) is generated using a Cox proportional hazards model. p-values in FIGS. 2A and 2B are calculated for these hazard ratios, based on testing the null hypothesis that the hazard ratio in each case equals 1.

FIGS. 2C-2D further elaborate the involvement of the HIF pathway in the etiology of aging-related morbidity. FIG. 2C shows that lower levels of HIF-PH (x axis) improve longevity (living ≥85 years) and physical function (good mobility ≥85 years) as depicted by increased probability of good outcome (y axis). FIG. 2D shows that higher levels of HIF1α (x axis) improve longevity (living ≥85 years) and physical function (good mobility ≥85 years) as depicted by increased probability of good outcome (y axis).

FIGS. 3A-3B show an activity wheel monitoring experimental set up for C57BL/6 mice and representative activity data. FIG. 3A shows a C57BL/6 mouse on an activity wheel in its cage; the wheel wirelessly transmits running activity data to a computer for analysis. FIG. 3B shows a running activity validation plot for evaluating frailty endpoints. C57BL/6 mice of different ages were tested in running wheel cages as in FIG. 3A, and group mean activity is plotted. The validation demonstrates robust differences in voluntary wheel activity as assessed by in-cage automated wheel monitors between young (3-5 months), middle aged (12 months), and old aged (18 months) animals.

FIG. 4A shows no significant differences in hemoglobin concentrations on day zero, before treatment, in 23 month old and 27 month old C57BL/6 mice in the experimental groups (“ctl 23 m”=23 month old control; “TP 23m”=TP-518 (BGE-117)-treated 23 month old mice; “ctl 27m”=27 month old control; “TP 27m”=TP-518 (BGE-117)-treated 27 month old mice.) FIG. 4B shows statistically significant differences (p<0.05 for “*” and p<0.01 for “***”) in hemoglobin concentrations in BGE-117 treated 27-month old mice versus 27 month old mice administered vehicle alone (control) after 14 days of treatment, with significant increase of hemoglobin levels in the BGE-117 treated group, and in hemoglobin concentrations in BGE-117 treated 23-month old mice versus 23 month old mice administered vehicle alone (control) after 14 days of treatment, with significant increase of hemoglobin levels in the BGE-117 treated group.

FIG. 5 shows that BGE-117 improves hemoglobin levels in old mice.

FIG. 6 shows that BGE-117 improves voluntary activity levels in old mice.

FIGS. 7A-7B shows differences in hemoglobin concentration between young (3-6-month old mice) and old C57BL/6 mice (23 months old or 27 months old mice). FIG. 7A presents data showing that young (3-6-month old mice) mice have a mean hemoglobin of 15.79 g/dL, and 23-month old mice have a mean hemoglobin concentration of 13.71 g/dL. FIG. 7B presents data showing that young (3-6-month old mice) mice have a mean hemoglobin concentration of 15.79 g/dL vs 27-month old mice having a mean hemoglobin concentration of 11.46 g/dL. Significance levels are noted.

FIGS. 8A-8D show inflammatory cytokine elevations in blood samples collected from the submandibular vein in old C57BL/6 mice (23 or 27-month old) compared to young (3-6-month-old) animals via Luminex 5-PLEX assays (lxsamsm-05). FIG. 8A shows very significant differences in TNFα blood levels between young and 23-month old mice. FIG. 8B shows very significant differences in TNFα blood levels between young and 27-month-old mice. FIG. 8C shows significant differences in IL-6 blood levels between young (3-6-month-old) and 23-month-old mice. FIG. 8D shows very significant differences in IL-6 blood levels between young and 27-month-old mice. LLOQ refers to the lower limit of quantification; below this limit, the results become qualitative (the relative position in relation to other data points is reliable, but the absolute concentration is not as well defined).

FIGS. 9A-9B show that BGE-117 improves hemoglobin levels in old mice with elevated levels of TNFα as compared to control (untreated) old mice with elevated levels of TNFα. 23-month-old C57BL/6 mice with elevated TNFα as assessed via Luminex 5-PLEX assays (lxsamsm-05) and as defined by having TNFα concentration at least 150% of the highest young value in our young cohort (see FIGS. 8A-8D) were used. Blood samples were taken from the submandibular vein before BGE-117 treatment, and again after two weeks of BGE-117 treatment. Hemoglobin was analyzed. FIG. 9A shows no significant change in hemoglobin levels in control animals. FIG. 9B shows significant increases in hemoglobin levels in animals treated with BGE-117, despite elevated TNFα.

FIGS. 10A-10B show that BGE-117 improves hemoglobin levels in old mice with elevated levels of IL-6 as compared to control (untreated) old mice with elevated levels of IL-6. 23 month old C57BL/6 mice with elevated IL-6 as assessed via Luminex 5-PLEX assays (lxsamsm-05) and as defined by having IL-6 concentration at least 150% of the highest young value in our young cohort (see FIGS. 8A-8D) were used. Blood samples were taken from the submandibular vein before BGE-117 treatment, and again after two weeks of BGE-117 treatment. Hemoglobin was analyzed. Highly significant improvements in hemoglobin levels were found in animals treated with BGE117 despite elevated IL-6 (FIG. 10B), whereas animals administered vehicle alone showed no changes (FIG. 10A).

FIGS. 11A-11B show that BGE-117 improves hemoglobin levels in old mice with elevated levels of TNFα as compared to control (untreated) old mice with elevated levels of TNFα. In this experiment, 27-month-old C57BL/6 mice with elevated TNFα as assessed via Luminex 5-PLEX assays (lxsamsm-05) and as defined by having TNFα concentration at least 150% of the highest young value in our young cohort (see FIGS. 8A-8D) were used. Blood samples were taken from the submandibular vein before BGE-117 treatment, and again after two weeks of BGE-117 treatment. Hemoglobin was analyzed. Significant improvements in hemoglobin levels were found in animals treated with BGE-117, despite elevated TNFα (FIG. 11B); control animals showed no significant change in hemoglobin levels (FIG. 11A).

FIGS. 12A-12B shows that BGE-117 improves hemoglobin levels in old mice with elevated levels of IL-6 as compared to control (untreated) old mice with elevated levels of IL-6. 27-month old C57BL/6 mice with elevated IL-6 as assessed via Luminex 5-PLEX assays (lxsamsm-05) and as defined by having IL-6 concentration at least 150% of the highest young value in our young cohort (see FIG. 8A-8D) were used. Blood samples were taken from the submandibular vein before BGE-117 treatment and again after two weeks of BGE117 treatment. Hemoglobin was analyzed. Significant improvements in hemoglobin levels were found in animals treated with BGE117 despite elevated IL-6 (FIG. 12B), whereas no change in hemoglobin levels was observed in control mice (FIG. 12A).

FIG. 13A-13B show that Roxadustat showed a trend, albeit not significant, of attenuating anemia (increasing hemoglobin levels) in mice with elevated TNFα as compared to control (untreated) old mice with elevated levels of TNFα. 27-month old C57BL/6 mice with elevated TNFα as assessed via Luminex and as defined by having TNFα concentration at least 150% of the highest young value in our young cohort (see FIGS. 8A-8D) were used. Blood samples were taken from the sub mandibular vein before roxadustat treatment and again after two weeks of roxadustat treatment. Hemoglobin was analyzed. No significant improvements in hemoglobin levels were found in animals treated with roxadustat, although a protective trend was observed (FIG. 13B).

FIGS. 14A-14B show that Roxadustat improves hemoglobin levels in old mice with elevated levels of IL-6 as compared control (untreated) old mice with elevated levels of IL-6. 27-month old C57BL/6 mice with elevated IL-6 as assessed via Luminex 5-PLEX assays (lxsamsm-05) and as defined by having IL-6 concentration at least 150% of the highest young value in our young cohort (see FIGS. 8A-8D) were used. Blood samples were taken from the submandibular vein before Roxadustat treatment and again after two weeks of Roxadustat treatment. Hemoglobin was analyzed. Although not statistically significant, 27-month old mice with elevated levels of IL-6 that were treated with Roxadustat showed improvement of anemia (increase in hemoglobin) (FIG. 14B), whereas untreated animals significantly declined in hemoglobin over the study period as they continued to age (p<0.05) (FIG. 14A).

FIG. 15 shows a phase-2 clinical trial design in elderly humans for BGE-117. Primary and Key endpoints include FACIT fatigue scale and hemoglobin levels.

FIG. 16 provides an overall study design flow schematic of the phase 2 clinical trial.

FIG. 17 shows that HIF-1α serum protein concentration decreases with age in human healthy aging cohorts of FIG. 2A.

FIG. 18 provides a heat map demonstrating a difference in HIF-1α signaling and gene expression between old (71-83 years old) humans and young (52-62 year old) humans from human healthy aging cohorts with clinical outcome data proprietary to those cohorts and proteomics data generated on archived samples. The difference of serum protein expression in the heat map shows that the level (e.g., upregulation or downregulation) of downstream HIF-1α genes are affected when the level of HIF-1α changes with age.

### 5. DETAILED DESCRIPTION

### 5.1. DEFINITIONS

Unless defined otherwise, all technical and scientific terms used herein have the meaning commonly understood by a person skilled in the art to which this disclosure belongs.

The terms “individual,” “host,” and “subject” are used interchangeably, and refer to an animal to be treated, including but not limited to humans and non-human primates; rodents, including rats and mice; bovines; equines; ovines; felines; and canines. “Mammal” means a member or members of any mammalian species. Non-human animal models, i.e., mammals, non-human primates, murines, lagomorpha, etc. may be used for experimental investigations.

The term “patient” refers to a human subject.

The terms “treating,” “treatment,” and grammatical variations thereof are used in the broadest sense understood in the clinical arts. Accordingly, the terms do not require cure or complete remission of disease, and encompass obtaining any clinically desired pharmacologic and/or physiologic effect, including improvement in physiologic measures associated with “normal”, non-pathologic, aging. Unless otherwise specified, “treating” and “treatment” do not encompass prophylaxis.

The phrase “therapeutically effective amount” refers to the amount of a compound that, when administered to a mammal or other subject for treating a disease, condition, or disorder, is sufficient to effect treatment of the disease, condition, or disorder. The “therapeutically effective amount” may vary depending on the compound, the disease and its severity and the age, weight, etc., of the subject to be treated.

The term “anemia of aging” refers to the development of anemia that is associated with aging. Anemia of aging includes, but is not limited to, unexplained anemia of aging (UAA), as described in Makipour et al., (Makipour et al., (2008) Unexplained Anemia in the Elderly. Semin Hematol. 45(4): pgs. 250-254), which is hereby incorporated by reference in its entirety. Anemia of aging also includes anemia of inflammation (AI) in old individuals, including anemia of inflammation (AI) in old individuals who have no diagnosed infection or cancer. In certain embodiments, anemia of aging is caused by one or more of: chronic disease, iron deficiency, Vitamin B12 deficiency, folate deficiency, gastrointestinal bleeding, and myelodysplastic syndrome. UAA is used synonymously herein with “unexplained anemia in the elderly” (UAE) and “spontaneous anemia of aging”.

The term “anemia of inflammation” refers to a type of anemia that affects people who have chronic conditions that cause inflammation, such as infections, autoimmune diseases, cancer link, and chronic kidney disease (CKD), and the like.

The term “pharmaceutically acceptable salt” refers to a salt that is acceptable for administration to a subject. Examples of pharmaceutically acceptable salts include, but are not limited to: mineral acid salts such as hydrochloride, hydrobromide, hydroiodide, phosphate, sulfate, and nitrate; sulfonic acid salts such as methanesulfonate, ethanesulfonate, benzenesulfonate, p-toluenesulfonate, and trifluoromethanesulfonate; organic acid salts such as oxalate, tartrate, citrate, maleate, succinate, acetate, trifluoroacetate, benzoate, mandelate, ascorbate, lactate, gluconate, and malate; amino acid salts such as glylcine salt, lysine salt, arginine salt, ornithine salt, glutamate, and aspartate; inorganic salts such as lithium salt, sodium salt, potassium salt, calcium salt, and magnesium salt; and salts with organic bases such as ammonium salt, triethylamine salt, diisopropylamine salt, and cyclohexylamine salt. The term “salt(s)” as used herein encompass hydrate salt(s).

Other examples of pharmaceutically salts include anions of the compounds of the present disclosure compounded with a suitable cation. For therapeutic use, salts of the compounds of the present disclosure can be pharmaceutically acceptable. However, salts of acids and bases that are non-pharmaceutically acceptable may also find use, for example, in the preparation or purification of a pharmaceutically acceptable compound.

Compounds included in the present compositions and methods that are basic in nature are capable of forming a wide variety of salts with various inorganic and organic acids. The acids that can be used to prepare pharmaceutically acceptable acid addition salts of such basic compounds are those that form non-toxic acid addition salts, i.e., salts containing pharmacologically acceptable anions, including but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts.

Compounds included in the present compositions and methods that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

The compounds of the present disclosure have an asymmetric center or asymmetric centers in certain cases, where they give rise to a variety of optical isomers. Therefore, the compounds of the present disclosure can exist as separate optical isomers (R) and (S), or as a racemate or an (RS) mixture. In the case of compounds having two or more asymmetric centers, they give rise to diastereomers due to their respective optical isomerisms. The compounds of the present disclosure encompass mixtures that comprise all these types of isomer in any proportions. For example, diastereomers can be separated by methods well known to those skilled in the art, say, fractional crystallization, and optically active forms can be obtained by techniques in organic chemistry that are well known for this purpose. In addition, the compounds of the present disclosure sometimes give rise to geometrical isomers such as cis- and trans-forms. Further in addition, the compounds of the present disclosure may have tautomerism to give rise to a variety of tautomers. The compounds of the present disclosure encompass the-above mentioned isomers, as well as mixtures comprising those isomers in any proportions.

Furthermore, if the compounds of the present disclosure or salts thereof form hydrates or solvates, these are also included in the scope of the compounds of the present disclosure or salts thereof.

Compounds included in the present compositions and methods that include a basic or acidic moiety can also form pharmaceutically acceptable salts with various amino acids. The compounds of the disclosure can contain both acidic and basic groups; for example, one amino and one carboxylic acid group. In such a case, the compound can exist as an acid addition salt, a zwitterion, or a base salt.

Ranges: throughout this disclosure, various aspects of the disclosure are presented in a range format. Ranges include the recited endpoints. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6, should be considered to have specifically disclosed subranges such as from 1 to 3, from 1 to 4, from 1 to 5, from 2 to 4, from 2 to 6, from 3 to 6, etc. as well as individual number within that range, for example, 1, 2, 3, 4, 5, 5.3, and 6. This applies regardless of the breadth of the range.

In this disclosure, “comprises,” “comprising,” “containing,” “having,” “includes,” “including”, and linguistic variants thereof have the meaning ascribed to them in U.S. Patent law, permitting the presence of additional components beyond those explicitly recited.

Unless specifically stated or apparent from context, as used herein the term “or” is understood to be inclusive.

Unless specifically stated or apparent from context, as used herein, the terms “a”, “an”, and “the” are understood to be singular or plural. That is, the articles “a” and “an” are used herein to refer to one or to more than one (i.e., to at least one) of the grammatical object of the article. By way of example, “an element” means one element or more than one element.

Unless specifically stated or otherwise apparent from context, as used herein the term “about” is understood as within range of normal tolerance in the art, for example within 2 standard deviations of the mean, and is meant to encompass variations of ±20% or ±10%, more preferably ±5%, even more preferably ±1%, and still more preferably ±0.1% from the stated value. Where a percentage is provided with respect to an amount of a component or material in a composition, the percentage should be understood to be a percentage based on weight, unless otherwise stated or understood from the context.

It should be understood that the order of steps or order for performing certain actions is immaterial so long as the present disclosure remain operable. Moreover, two or more steps or actions can be conducted simultaneously.

The terms “pharmaceutically acceptable excipient,” “pharmaceutically acceptable diluent,” “pharmaceutically acceptable carrier,” and “pharmaceutically acceptable adjuvant” are used interchangeably and refer to an excipient, diluent, carrier, or adjuvant that is useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. The phrase “pharmaceutically acceptable excipient” includes both one and more than one such excipient, diluent, carrier, and/or adjuvant.

As used herein, the term “sustained release”, “delayed release”, and “controlled release” refer to prolonged or extended release of the therapeutic agent or API of the controlled release pharmaceutical formulation. These terms may further refer to composition which provides prolonged or extended duration of action, such as pharmacokinetics (PK) parameters of a pharmaceutical composition comprising a therapeutically effective amount of the active pharmaceutical ingredient as described herein.

Generally, reference to or depiction of a certain element such as hydrogen or H is meant to include all isotopes of that element. For example, if an R group is defined to include hydrogen or H, it also includes deuterium and tritium. Compounds comprising radioisotopes such as tritium, ¹⁴C, ³²P and ³⁵S are thus within the scope of the present technology. Procedures for inserting such labels into the compounds of the present technology will be readily apparent to those skilled in the art based on the disclosure herein.

Unless the specific stereochemistry is expressly indicated, all chiral, diastereomeric, and racemic forms of a compound are intended. Thus, compounds described herein include enriched or resolved optical isomers at any or all asymmetric atoms as are apparent from the depictions. Racemic mixtures of R-enantiomer and S-enantiomer, and enantio-enriched stereomeric mixtures comprising of R- and S-enantiomers, as well as the individual optical isomers can be isolated or synthesized so as to be substantially free of their enantiomeric or diastereomeric partners, and these stereoisomers are all within the scope of the present technology.

The “halogen atom” refers to a fluorine atom, a chlorine atom, a bromine atom, and an iodine atom.

The “C₁₋₃ alkyl” refers to linear or branched alkyl having one to three carbon atoms. Specifically, methyl, ethyl, n-propyl, and isopropyl are referred to.

The “C₁₋₄ alkyl” refers to linear or branched alkyl having one to four carbon atoms. Specifically, methyl, ethyl, n-propyl, isopropyl, n-butyl, isobutyl, sec-butyl, and tert-butyl are referred to.

The “C₁₋₆ alkyl” refers to linear or branched alkyl having one to six carbon atoms, and examples include methyl, ethyl, n-propyl, isopropyl, n-butyl, isobutyl, sec-butyl, tert-butyl, n-pentyl, isopentyl, neopentyl, 2-methylbutyl, n-hexyl, isohexyl, etc.

The “halo-C₁₋₄ alkyl” refers to linear or branched alkyl having one to four carbon atoms, with substitution by a halogen atom. The number of substitutions by a halogen atom is preferably from one to three, and a preferred halogen atom is a fluorine atom. Examples include monofluoromethyl, difluoromethyl, trifluoromethyl, 1-fluoroethyl, 1,1-difluoroethyl, 2-fluoroethyl, 2-fluoro-2-methylpropyl, 2,2-difluoropropyl, 1-fluoro-2-methylpropan-2-yl, 1,1-difluoro-2-methylpropan-2-yl, etc.

The “halo-C₁₋₆ alkyl” refers to linear or branched alkyl having one to six carbon atoms, with substitution by a halogen atom. The number of substitutions by a halogen atom is preferably from one to five, and a preferred halogen atom is a fluorine atom. Examples include monofluoromethyl, difluoromethyl, trifluoromethyl, 1-fluoroethyl, 1,1-difluoroethyl, 1,1,2,2,2-pentafluoroethyl, 2-fluoroethyl, 2-fluoro-2-methylpropyl, 2,2-difluoropropyl, 1-fluoro-2-methylpropan-2-yl, 1,1-difluoro-2-methylpropan-2-yl, 1-fluoropentyl, 1-fluorohexyl, etc.

The “C₃₋₆ cycloalkane” refers to cyclic alkane having three to six carbon atoms. Examples include cyclopropane, cyclobutane, cyclopentane, and cyclohexane.

The “C₃₋₈ cycloalkane” refers to cyclic alkane having three to eight carbon atoms. Examples include cyclopropane, cyclobutane, cyclopentane, cyclohexane, cycloheptane, and cyclooctane.

The “C₃₋₈ cycloalkyl” refers to cyclic alkyl having three to eight carbon atoms. Examples include cyclopropyl, cyclobutyl, cyclopentyl, cyclohexyl, cycloheptyl, and cyclooctyl.

The “C₃₋₈ cycloalkenyl” refers to cyclic alkenyl having three to eight carbon atoms. Examples include cyclopropenyl, cyclobutenyl, cyclopentenyl, cyclohexenyl, cycloheptenyl, and cyclooctenyl.

The “4- to 8-membered saturated heterocycle containing an oxygen atom” refers to a 4- to 8-membered monocylic saturated heterocycle containing one oxygen atom in the ring. Examples include oxetane, tetrahydrofuran, tetrahydropyran, etc.

The “4- to 8-membered saturated heterocycle containing a nitrogen atom” refers to a 4 to 8-membered monocylic saturated heterocycle containing one nitrogen atom in the ring. Examples include azetidine, pyrrolidine, piperidine, etc.

The “4- to 8-membered saturated heterocyclyl containing a nitrogen atom” refers to a 4 to 8-membered monocytic saturated heterocyclic group containing one nitrogen atom in the ring. Examples include azetidinyl, pyrrolidinyl, piperidinyl, etc.

The “C₁₋₃ alkoxy” refers to linear or branched alkoxy having one to three carbon atoms. Specifically, methoxy, ethoxy, n-propoxy, and isopropoxy are referred to.

The “C₁₋₆ alkoxy” refers to linear or branched alkoxy having one to six carbon atoms. Examples include methoxy, ethoxy, n-propoxy, isopropoxy, n-butoxy, isobutoxy, sec-butoxy, tert-butoxy, n-pentyloxy, isopentyloxy, neopentyloxy, 2-methylbutoxy, n-hexyloxy, isohexyloxy, etc.

The “halo-C₁₋₆ alkoxy” refers to linear or branched alkoxy having one to six carbon atoms, with substitution by a halogen atom. The number of substitutions by a halogen atom is preferably from one to five, and a preferred halogen atom is a fluorine atom. Examples include monofluoromethoxy, difluoromethoxy, trifluoromethoxy, 1-fluoroethoxy, 1,1-difluoroethoxy, 1,1,2,2-tetrafluoroethoxy, 2-fluoroethoxy, 2,2,2-trifluoroethoxy, 3,3,3-trifluoropropoxy, 1,3-difluoropropan-2-yloxy, 2-fluoro-2-methylpropoxy, 2,2-difluoropropoxy, 1-fluoro-2-methylpropan-2-yloxy, 1,1-difluoro-2-methylpropan-2-yloxy, 4,4,4-trifluorobutoxy, etc.

The “C₁₋₆ alkenyloxy” refers to a group of such a structure that oxy is bound to linear or branched alkenyl having two to six carbon atoms. Examples include ethenyloxy, (E)-prop-1-en-1-yloxy, (Z)-prop-1-en-1-yloxy, prop-2-en-1-yloxy, (Z)-but-2-en-1-yloxy, (Z)-pent-3-en-1-yloxy, (Z)-hex-4-en-1-yloxy, (Z)-hept-5-en-1-yloxy, and (Z)-oct-6-en-1-yloxy, etc.

The “C₃₋₈ cycloalkoxy” refers to cyclic alkoxy having three to eight carbon atoms. Examples include cyclopropoxy, cyclobutoxy, cyclopentyloxy, cyclohexyloxy, cycloheptyloxy, and cyclooctyloxy.

The “di-C₁₋₃ alkylamino” refers to amino having the aforementioned “C₁₋₃ alkyl” as two substituents which are the same or different. Examples include dimethylamino, diethylamino, di(n-propyl)amino, di(isopropyl)amino, ethylmethylamino, methyl(n-propyl)amino, etc.

The “di-C₁₋₆alkylamino” refers to amino having the aforementioned “C₁₋₆ alkyl” as two substituents which are the same or different. Examples include dimethylamino, diethylamino, di(n-propyl)amino, di(isopropyl)amino, ethylmethylamino, methyl(n-propyl)amino, etc.

The “C₁₋₆ alkylcarbonyl” refers to a group of such a structure that carbonyl is bound to the aforementioned “C₁₋₆ alkyl”. Examples include methylcarbonyl, ethylcarbonyl, n-propylcarbonyl, isopropylcarbonyl, n-butylcarbonyl, isobutylcarbonyl, sec-butylcarbonyl, tert-butylcarbony, n-pentylcarbonyl, isopentylcarbonyl, neopentylcarbonyl, 2-methylbutylcarbonyl, n-hexylcarbonyl, isohexylcarbonyl, etc.

The “mono-C₁₋₆ alkylaminocarbonyl” refers to a group of such a structure that carbonyl is bound to amino having the aforementioned “C₁₋₆ alkyl” as a single substituent. Examples include methylaminocarbonyl, ethylaminocarbonyl, n-propylaminocarbonyl, isopropylaminocarbonyl, n-butylaminocarbonyl, isobutylaminocarbonyl, sec-butylaminocarbonyl, tert-butylaminocarbonyl, n-pentylaminocarbonyl, n-hexylaminocarbonyl, etc.

The “di-C₁₋₆ alkylaminocarbonyl” refers to a group of such a structure that carbonyl is bound to amino having the aforementioned “C₁₋₆ alkyl” as two substituents which are the same or different. Examples include dimethylaminocarbonyl, di(n-propyl)aminocarbonyl, di(isopropyl)aminocarbonyl, ethylmethylaminocarbonyl, methyl(n-propyl)aminocarbonyl, etc.

The two C₁₋₆ alkyls in the di-C₁₋₆ alkylaminocarbonyl, together with the adjacent nitrogen atom, may optionally form a 4- to 8-membered saturated heterocycle containing a nitrogen atom.

The “C₁₋₆ alkylsulfanyl” refers to a group of such a structure that sulfanyl is bound to the aforementioned “C₁₋₆ alkyl”. Examples include methylsulfanyl, ethylsulfanyl, n-propylsulfanyl, isopropylsulfanyl, isobutylsulfanyl, n-hexylsulfanyl, etc.

The “C₁₋₆ alkylsulfonyl” is a group of such a structure that sulfonyl is bound to the aforementioned “C₁₋₆ alkyl”. Examples include methylsulfonyl, ethylsulfonyl, n-propylsulfonyl, isopropylsulfonyl, isobutylsulfonyl, n-hexyl sulfonyl, etc.

The “mono-C₁₋₆ alkylaminosulfonyl” refers to a group of such a structure that sulfonyl is bound to amino having the aforementioned “C₁₋₆ alkyl” as a single substituent. Examples include methylaminosulfonyl, ethylaminosulfonyl, n-propylaminosulfonyl, isopropylaminosulfonyl, n-butylaminosulfonyl, isobutylaminosulfonyl, sec-butylaminosulfonyl, tert-butylaminosulfonyl, n-pentylaminosulfonyl, n-hexylaminosulfonyl, etc.

The “di-C₁₋₆ alkylaminosulfonyl” refers to a group of such a structure that sulfonyl is bound to amino having the aforementioned “C₁₋₆ alkyl” as two substituents which are the same or different. Examples include dimethylaminosulfonyl, diethylaminosulfonyl, di(n-propyl)aminosulfonyl, di(isopropyl)aminosulfonyl, ethylmethylaminosulfonyl, methyl(n-propyl)aminosulfonyl, isopropyl(methyl)aminosulfonyl, etc.

The “C₁₋₄ alkanediyl” refers to a divalent hydrocarbon group of such a structure that one hydrogen atom has been removed from an alkyl group having one to four carbon atoms. Examples include methanediyl, ethane-1,1-diyl, ethane-1,2-diyl, propane-1,1-diyl, propane-1,2-diyl, propane-1,3-diyl, propane-2,2-diyl, butane-1,4-diyl, 2-methylpropane-1,2-diyl, etc. Among these, methanediyl, ethane-1,1-diyl, ethane-1,2-diyl, propane-1,1-diyl, propane-1,2-diyl, propane-1,3-diyl, and propane-2,2-diyl are C₁₋₃ alkanediyls.

The “C₁₋₆ alkanediyl” refers to a divalent hydrocarbon group of such a structure that one hydrogen atom has been removed from an alkyl group having one to six carbon atoms. Examples include methanediyl, ethane-1,1-diyl, ethane-1,2-diyl, propane-1,1-diyl, propane-1,2-diyl, propane-1,3-diyl, propane-2,2-diyl, butane-1,4-diyl, 2-methylpropane-1,2-diyl, pentane-1,5-diyl, hexane-1,6-diyl, etc.

The “C₃₋₆ cycloalkane-1,1-diyl” refers to a divalent cyclic hydrocarbon group of such a structure that one hydrogen atom has been removed from a cycloalkyl group having three to six carbon atoms. Examples include cyclopropane-1,1-diyl, cyclobutane-1,1-diyl, cyclopentane-1,1-diyl, and cyclohexane-1,1-diyl.

The “phenyl-C₁₋₃ alkyl” refers to the aforementioned “C₁₋₃ alkyl” having a phenyl group as a substituent. Examples include benzyl, phenethyl, and phenylpropyl.

The “C₃₋₈ cycloalkyl-C₁₋₃ alkylcarbonyl” refers to a group of such a structure that the aforementioned cycloalkyl group having three to eight carbon atoms binds a carbonyl group via the aforementioned C₁₋₃ alkyl. Examples include cyclopropylmethylcarbonyl, cyclopropylethylcarbonyl, cyclobutylmethylcarbonyl, cyclopentylmethylcarbonyl, cyclohexylmethylcarbonyl, etc.

The “phenyl-C₁₋₃ alkoxycarbonyl” refers to a group of such a structure that a phenyl group binds a carbonyl group via the aforementioned C₁₋₃ alkoxy. Examples include phenylmethoxycarbonyl, phenylethoxycarbonyl, and phenylpropoxycarbonyl

On the following pages, the compounds of the present disclosure are described in greater detail but it should be understood that the present disclosure is by no means limited to the following illustrations.

As described herein, the text refers to various embodiments of the present compounds, compositions, and methods. The various embodiments described are meant to provide a variety of illustrative examples and should not be construed as descriptions of alternative species. Rather, it should be noted that the descriptions of various embodiments provided herein may be of overlapping scope. The embodiments discussed herein are merely illustrative and are not meant to limit the scope of the present technology.

### 5.2. SURVIVAL PREDICTOR MODEL

Aspects of the present disclosure include a bioinformatics model that generally relates to building of survival predictor models that output a survival metric. Such survival metrics may relate to survival related observables, such as survival expectancy and/or risk of death. In various embodiments, survival predictor models may be built by selecting observables that relate to survival periods (“aging indicator”). Such aging indicators may comprise variables that correlate with all-cause mortality, such as certain clinical factors. In some embodiments, survival predictor models utilize one or a plurality of survival biomarkers together with one or more aging indicators to generate a survival metric.

A survival predictor model of the present disclosure is to examine the relationship between serum levels of HIF1α and HIF-PH, and future risk of all-cause mortality in human healthy aging cohorts, with clinical outcome data proprietary to those cohorts and proteomics data generated on archived samples, based on survival modeling. Additionally, the relationship between HIF1α or HIF-PH levels and mobility decline events (e.g., a decrease in ability of walking, stair-climbing, or transferring activities as shown by self-reported difficulty of these activities) is examined using a Cox proportional hazards model, with a hazard ratio and associated p-value generated for each of HIF1α and HIF-PH.

### 5.3. METHODS OF TREATING AGE-RELATED CONDITIONS

We applied bioinformatic and machine learning approaches to analyze human data using survival predictor models and discovered an association of baseline HIF1α pathway protein levels with future aging outcomes. In particular, we discovered that higher circulating levels of HIF1α are associated with decreased all-cause mortality (p=0.0029)—that is, greater longevity—and that higher circulating levels of HIF-PH, which triggers degradation of HIF-1α, are associated with increased all-cause mortality (p=0.0201). In addition, the analysis demonstrated that higher levels of HIF1α are associated with better future physical function, whereas higher levels of HIF-PH are associated with worse future physical function.

We then discovered that HIF-1α serum protein concentration decreases with age in human healthy aging cohorts, and that expression of known downstream target genes of HIF-1α are affected, in turn, by the decrease in HIF-1α as humans age.

Based on these discoveries, we tested an inhibitor of HIF prolyl hydroxylase, BGE-117, for effects in aged mice. BGE-117 (also known as TP0463518 and TP518) has the structure shown below:

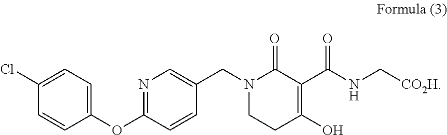 [SMILES: O=C(O)CNC(=O)C1=C(O)CCN(Cc2ccc(Oc3ccc(Cl)cc3)nc2)C1=O]

In a first set of experiments, we demonstrated that aged mice (27 months old) treated with BGE-117 exhibited a statistically significant (p<0.001) increase in voluntary activity as compared to age-matched controls, indicating a reduction in frailty and improved physical health.

In addition, we discovered that 27-month old mice treated with BGE-117 showed increased hemoglobin levels. Although BGE-117 was known to inhibit HIF-PH and increase erythropoietin (EPO) production in normal healthy human volunteers (Shinfuku et al., Am. J. Nephrol. 48(3):157-164 (2018)) and patients with chronic kidney disease, and has been shown to increase hemoglobin levels in 5/6 nephrectomized young rats (Kato et al., J. Pharmacol. Exp. Ther. 371:675-683 (2019), its effects on aged individuals with normal kidney function had not previously been reported.

We then discovered that aged mice (23-month old and 27-month old) spontaneously develop anemia of aging, and that this anemia is accompanied by elevated levels of inflammatory cytokines IL-6 and TNFα. This discovery suggested that an underlying etiology of the spontaneous anemia is anemia of inflammation. We then sought to determine whether the beneficial effects of BGE-117 would still be observed in aged mice with anemia, and in particular, anemia of inflammation, or instead is limited to aged mice with normal baseline hemoglobin levels and without elevated levels of inflammatory cytokines. Selecting mice in each age cohort that had high levels of inflammatory cytokines, we demonstrated that even in aged mice with anemia of inflammation, BGE-117 is effective in increasing hemoglobin level.

Next, we demonstrated that another HIF-PH inhibitor, roxadustat, is also capable of increasing hemoglobin in aged animals exhibiting spontaneous inflammation and anemia of inflammation, thus demonstrating that HIF-PH inhibitors as a class are effective in treating anemia in aged animals, including aged animals with anemia of inflammation.

Accordingly, in a first aspect, the present disclosure provides a method of treating an aging-related morbidity, comprising: administering a therapeutically effective amount of a hypoxia-inducible factor prolyl hydroxylase (HIF-PH) inhibitor to a human subject older than 40 years old who has, or is at risk for developing, an aging-related morbidity. In various embodiments, the diseases or conditions are selected from the group consisting of: anemia, anemia of inflammation (AI), including anemia with chronic kidney disease; anemia of aging, sarcopenia, frailty, muscle injury, and ischemic damage. In some embodiments, the disease or condition is one for which tissue regeneration or wound healing or therapeutic. In some embodiments, the disease or condition is fibrosis.

The HIF-PH inhibitor can inhibit HIF-PH directly or indirectly, competitively or non-competitively. As further described below, in some embodiments the inhibitor of HIF-PH is a compound described in U.S. Pat. No. 9,422,240, which is hereby incorporated by reference in its entirety.

### 5.3.1. Subject to Be Treated

5.3.1.1 Subject Age

In some embodiments, the human subject (patient) is older than 40 years of age. In certain embodiments, the patient is older than 50 years of age. In certain embodiments, the patient is older than 51, 52, 53, 54, 55, 56, 57, 58, 59, 60, 61, 62, 63, 64, 65, 66, 67, 68, 69, 70, 71, 72, 73, 74, or 75 years of age. In certain embodiments, the patient is older than 76, 77, 78, 79, 80, 81, 82, 82, 84, 85, 86, 87, 88, 89 or 90 years of age. In various embodiments, the patient is 40-50 years old, 50-60 years old, 52-62 years old, 63-70 years old, 60-70 years old, 70-80 years old, or 80-90 years old. In certain embodiments, the patient is 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 years old.

5.3.1.2 Pre-Treatment Muscle Force and Volume

In some embodiments, before treatment with an HIF-PH inhibitor in the methods described herein (pre-treatment), the subject has a reduction in muscle force associated with aging as compared to muscle force of a human subject that is younger than 40 years old.

In some embodiments, the patient has a reduction in lower limb muscle mass associated with aging as compared to lower limb muscle mass of a human subject that is younger than 40 years old.

In some embodiments, the patient has a reduction in upper limb muscle mass associated with aging as compared to upper limb muscle mass of a human subject that is younger than 40 years old.

In some embodiments, the patient has a reduction in muscle volume associated with aging as compared to muscle volume of a human subject that is younger than 40 years old.

In some embodiments, the muscle volume is the muscle volume of one or more upper limb muscles selected from the group consisting of: shoulder abductors, shoulder adductors, elbow flexors, elbow extensors, wrist flexors, and wrist extensors.

5.3.1.3 Pre-Treatment Capillary Density

In some embodiments, before treatment with an HIF-PH inhibitor in the methods described herein (pre-treatment), the subject has a reduction in capillary density as compared to muscle force of a human subject that is younger than 40 years old.

In some embodiments, the patient has a reduction in capillary density as compared to muscle force of a human subject that is younger than 50 years old.

In some embodiments, the patient has a reduction in capillary density as compared to muscle force of a human subject that is younger than 60 years old.

5.3.1.4 Age-Related Conditions

In some embodiments, before treatment with an HIF-PH inhibitor in the methods described herein (pre-treatment), the subject does not have a renal disease. In certain embodiments, the patient does not have a chronic kidney disease (CKD). In particular embodiments, the patient does not have CKD stage 3-5. In particular embodiments, the patient is not on hemodialysis.

In some embodiments, the patient has a renal disease. In certain embodiments, the patient has a chronic kidney disease. In particular embodiments, the patient has CKD stage 3-5. In certain embodiments, the patient is on hemodialysis. In certain embodiments, the patient is not on hemodialysis.

In some embodiments, the patient does not have anemia.

In some embodiments, the patient has age-related AI.

In some embodiments, the patient has anemia. In particular embodiments, the anemia is associated with chronic kidney disease. In particular embodiments, the patient has anemia of aging. Anemia of aging includes, but is not limited to, unexplained anemia of aging (UAA), and AI, e.g., as described in Makipour, et al., (Makipour et al., (2008) Unexplained Anemia in the Elderly. Semin Hematol. 45(4): pgs. 250-254), which is hereby incorporated by reference in its entirety.

In some embodiments, the patient has transfusion-dependent age-associated anemia. Transfusion-dependent age-associated anemia can be described in, for example, Beyer et al., (Beyer et al., (2010) Anemia and transfusions in geriatric patients: a time for evaluation. Hematology. 15(2): pgs. 116-121), which is hereby incorporated by reference in its entirety.

In particular embodiments, unexplained anemia of aging can include, but is not limited to, one or more of: age-associated decline in renal endocrine function resulting in a reduced erythropoietin response; age-related reduction in androgen levels, accounting for a decline in hemoglobin level of up to 1 g/dL; age-related increase in inflammatory markers, such as anemia associated to age-associated cytokine dysregulation (e.g., IL-6 and TNFα) by an increase in elevated cytokine levels by mechanisms for inflammation such as inhibition of erythropoietin and induction of hepcidin; age-associated contribution of hematopoietic stem cell proliferative capacity; and early myelodysplasia (MDS) presenting as anemia without associated white blood cell or platelet features.

In some embodiments, the age-related morbidity is frailty. In certain embodiments, the patient has sarcopenia.

In some embodiments, the patient has not been diagnosed with any disease except age-related frailty.

In some embodiments, the age-related morbidity is fatigue. In certain embodiments, fatigue is measured using a patient recorded outcome (PRO). In certain embodiments, fatigue is measured using a FACIT fatigue scale.

In some embodiments, the age-related morbidity is tissue injury. In certain embodiments, the patient has a muscle injury.

In some embodiments, the patient has a muscle-aging-related condition. In certain embodiments, the muscle-aging related condition is hip fracture/hip fracture functional recovery. In certain embodiments, the muscle-aging-related condition is Intensive-Care-Unit syndrome. In certain embodiments, the muscle-aging related condition is Intensive Care Unit-acquired weakness. Additional muscle-aging related conditions include, but are not limited to, muscle-aging related conditions described in Beaupre et al., (2014), and Inoue et al., 2019 (Beaupre et al., (2014) Maximising functional recovery following hip fracture in frail seniors. Best Pract. Res. Clin. Rheumatol. 27(6): pgs. 771-788; Inoue et al., (2019) Post-intensive care syndrome: its pathophysiology, prevention, and future directions. Acute Med. Surg. 6(3): pgs. 233-246), each of which are hereby incorporated by reference in their entireties.

In certain embodiments, administering a HIF-PH inhibitor to the patient having a tissue injury promotes tissue regeneration in the patient. In certain embodiments, administering a HIF-PH inhibitor to a patient having a tissue injury promotes wound healing in the patient.

In some embodiments, the age-related condition is ischemic damage.

5.3.1.5 Pre-Treatment Characteristics

In some embodiments, before treatment with an HIF-PH inhibitor in the methods described herein (pre-treatment), the subject has one or more signs and/or symptoms of an age-related morbidity.

5.3.1.5.1 Pre-Treatment CRP and IL-6 Levels

In some embodiments, the patient has an IL-6-mediated inflammation.

In some embodiments, the patient has elevated pre-treatment levels of C-reactive protein (CRP).

In some embodiments, the patient has a pre-treatment CRP level at least 2 mg/L. In some embodiments, the patient has a pre-treatment CRP level at least 2 mg/L, 2.5 mg/L, 3 mg/L, 3.5 mg/L, 4 mg/L, 4.5 mg/L, or 5 mg/L. In some embodiments, the patient has pre-treatment CRP levels at least 7.5 mg/L, 10 mg/L, 12.5 mg/L, or 15 mg/L. In various embodiments, the patient has a pre-treatment CRP level at least 2 mg/L. In various embodiments, the patient has a pre-treatment CRP level at least 2.5 mg/L. In various embodiments, the patient has a pre-treatment CRP level at least 5 mg/L. In various embodiments, the patient has a pre-treatment CRP level at least 7.5 mg/L. In various embodiments, the patient has a pre-treatment CRP level at least 10 mg/L. In various embodiments, the patient has a pre-treatment CRP level at least 12.5 mg/L. In various embodiments, the patient has a pre-treatment CRP level at least 15 mg/L.

In some embodiments of the methods described herein, the patient has elevated pre-treatment serum levels of IL-6.

In some embodiments, the patient has a pre-treatment IL-6 level of at least 2 pg/ml. In various embodiments, the patient has a pre-treatment IL-6 level of at least 2 pg/ml, at least 3 pg/ml, at least 4 pg/ml, at least 5 pg/ml, at least 6 pg/ml, at least 7 pg/ml, at least 8 pg/ml, at least 9 pg/ml, at least 10 pg/ml, at least 11 pg/ml, at least 12 pg/ml, at least 13 pg/ml, at least 14 pg/ml, or at least 15 pg/ml. In certain embodiments, the patient has a pre-treatment IL-6 level of at least 2 pg/ml. In certain embodiments, the patient has a pre-treatment IL-6 level of at least 2.5 pg/ml. In certain embodiments, the patient has a pre-treatment IL-6 level of at least 4 pg/ml. In certain embodiments, the patient has a pre-treatment IL-6 level of at least 5 pg/ml. In certain embodiments, the patient has a pre-treatment IL-6 level of at least 7.5 pg/ml. In certain embodiments, the patient has a pre-treatment IL-6 level of at least 10 pg/ml. In certain embodiments, the patient has a pre-treatment IL-6 level of at least 12.5 pg/ml. In certain embodiments, the patient has a pre-treatment IL-6 level of at least 15 pg/ml.

In some embodiments, the patient has elevated pre-treatment levels of CRP and elevated pre-treatment IL-6 levels. In certain embodiments, the patient has a pre-treatment IL-6 level of at least 2 pg/ml and a pre-treatment CRP level at least 2 mg/L. In certain embodiments, the patient has a pre-treatment IL-6 level of at least 2 pg/ml and a pre-treatment CRP level at least 2.5 mg/L. In certain embodiments, the patient has a pre-treatment IL-6 level of at least 2 pg/ml and a pre-treatment CRP level at least 5 mg/L. In certain embodiments, the patient has a pre-treatment IL-6 level of at least 2 pg/ml and a pre-treatment CRP level at least 10 mg/L. In certain embodiments, the patient has a pre-treatment serum IL-6 level of at least 4 pg/ml and a pre-treatment CRP level at least 2 mg/L. In certain embodiments, the patient has a pre-treatment IL-6 level of at least 4 pg/ml and a pre-treatment CRP level at least 2.5 mg/L. In certain embodiments, the patient has a pre-treatment IL-6 level of at least 4 pg/ml and a pre-treatment CRP level at least 5 mg/L. In certain embodiments, the patient has a pre-treatment IL-6 level of at least 4 pg/ml and a pre-treatment CRP level at least 10 mg/L. In certain embodiments, the patient has a pre-treatment IL-6 level of at least 5 pg/ml and a pre-treatment CRP level at least 2 mg/L. In certain embodiments, the patient has a pre-treatment IL-6 level of at least 5 pg/ml and a pre-treatment CRP level at least 2.5 mg/L. In certain embodiments, the patient has a pre-treatment IL-6 level of at least 5 pg/ml and a pre-treatment CRP level at least 5 mg/L. In certain embodiments, the patient has a pre-treatment IL-6 level of at least 5 pg/ml and a pre-treatment CRP level at least 10 mg/L. In certain embodiments, the patient has a pre-treatment IL-6 level of at least 10 pg/ml and a pre-treatment CRP level at least 2 mg/L. In certain embodiments, the patient has a pre-treatment IL-6 level of at least 10 pg/ml and a pre-treatment CRP level at least 2.5 mg/L. In certain embodiments, the patient has a pre-treatment IL-6 level of at least 10 pg/ml and a pre-treatment CRP level at least 5 mg/L. In certain embodiments, the patient has a pre-treatment IL-6 level of at least 10 pg/ml and a pre-treatment CRP level at least 10 mg/L.

5.3.1.5.2 Pre-Treatment TNFα Levels

In some embodiments, the patient has TNFα-mediated inflammation.

In some embodiments, the patient has a pre-treatment TNFα level of at least 5 pg/ml. In various embodiments, the patient has a pre-treatment TNFα level of at least 5 pg/ml, at least 5.5 5 pg/ml, at least 6 pg/ml, at least 6.5 pg/ml, at least 7 pg/ml, at least 7.5 pg/ml, at least 8 pg/ml, at least 8.5 pg/ml, at least 9 pg/ml, at least 9.5 pg/ml, at least 10 pg/ml, at least 10.5 pg/ml, at least 11 pg/ml, at least 11.5 pg/ml, at least 12 pg/ml, at least 12.5 pg/ml, at least 13 pg/ml, at least 13.5 pg/ml, at least 14 pg/ml, at least 14.5 pg/ml, or at least 15 pg/ml.

In various embodiments, the patient has a a pre-treatment TNFα level of 5 pg/ml. In various embodiments, the patient has a a pre-treatment TNFα level of 5.5 pg/ml. In various embodiments, the patient has a a pre-treatment TNFα level of 6 pg/ml. In various embodiments, the patient has a a pre-treatment TNFα level of 6.5 pg/ml. In various embodiments, the patient has a a pre-treatment TNFα level of 7 pg/ml. In various embodiments, the patient has a a pre-treatment TNFα level of 7.5 pg/ml. In various embodiments, the patient has a pre-treatment TNFα level of 8 pg/ml. In various embodiments, the patient has a a pre-treatment TNFα level of 8.5 pg/ml. In various embodiments, the patient has a a pre-treatment TNFα level of 9 pg/ml. In various embodiments, the patient has a a pre-treatment TNFα level of 9.5 pg/ml. In various embodiments, the patient has a a pre-treatment TNFα level of 10 pg/ml.

5.3.1.5.3 Pre-Treatment Estimated Glomerular Filtration Rate (eGFR) Levels

In some embodiments, the patient has a pre-treatment estimated glomerular filtration rate (eGFR) of greater than 30 ml/min. In various embodiments, the patient has a pretreatment eGFR of greater than 35, 40, 45, 50, 55, 60, 65, 70, 75, or 80 ml/min. In various embodiments, the patient has an eGFR of greater than 40 ml/min. In various embodiments, the patient has an eGFR of greater than 50 ml/min. In various embodiments, the patient has an eGFR of greater than 60.

5.3.1.5.4 Pre-Treatment Serum Ferritin (SF) Levels and Tumor Inflammation Signature (TIS)

In some embodiments, the patient has pre-treatment serum ferritin (SF) levels greater than 100. In various embodiments, the patient has pre-treatment SF levels greater than 105, 110, 115, 120, 125, 130, 135, 140, 145, 150, 155, 160, 165, 170, 175, 180, 185, 190, 195, or 200. In various embodiments, the patient has pre-treatment SF levels of 100, 105, 110, 115, 120, 125, 130, 135, 140, 145, 150, 155, 160, 165, 170, 175, 180, 185, 190, 195, or 200.

In some embodiments, the patient has pre-treatment Tumor Inflammation Signature (TIS) of greater than 20%. In various embodiments, the patient has pre-treatment TIS of greater than 25%, 30%, 35%, 40%, 45%, 50%, 55%, 60%, 65%, or 70%. In various embodiments, the patient has pre-treatment TIS levels of 20%, 25%, 30%, 35%, 40%, 45%, 50%, 55%, 60%, 65%, or 70%.

In various embodiments, the patient has pre-treatment serum ferritin (SF) levels greater than 100, 105, 110, 115, 120, 125, 130, 135, 140, 145, 150, 155, 160, 165, 170, 175, 180, 185, 190, 195, or 200; and pre-treatment TIS of greater than 25%, 30%, 35%, 40%, 45%, 50%, 55%, 60%, 65%, or 70%.

5.3.1.5.5 Pre-Treatment Folate and B12 Levels

In some embodiments, the patient has normal folate and/or B12 levels.

In various embodiments, the patient has pre-treatment folate levels of red blood cells ranging from 2 to 10 ng/mL. In various embodiments, the patient has pre-treatment folate levels of blood plasma ranging from 140 to 960 ng/mL

In various embodiments, the patient has pre-treatment B12 levels ranging from 200 to 900 ng/ml.

5.3.1.5.6 Pre-Treatment Hb Levels

In certain embodiments, the patient has a pre-treatment hemoglobin level above 12 g/dL. In various embodiments, the patient has a pre-treatment hemoglobin level above 12 g/dL, above 12.1 g/dL, above 12.2 g/dL, above 12.3 g/dL, above 12.4 g/dL, above 12.5 g/dL, above 12.6 g/dL, above 12.7 g/dL, above 12.8 g/dL, above 12.9 g/dL, or above 13.0 g/dL. In various embodiments, the patient has a pre-treatment hemoglobin level above 13 g/dL, above 14 g/dL, above 15 g/dL, above 16 g/dL, above 17 g/dL, above 18 g/dL, above 19 g/dL, above 20 g/dL, above 21 g/dL, above 22 g/dL, above 23 g/dL, above 24 g/dL, above 25 g/dL, above 26 g/dL, above 27 g/dL, above 28 g/dL, above 29 g/dL, or above 30 g/dL. In certain embodiments, the patient has a pre-treatment hemoglobin level below 12 g/dL, below 11 g/dL, below 10 g/dL, below 9 g/dL, below 8 g/dL, below 7 g/dL, below 6 g/dL, below 5 g/dL, below 4 g/dL, below 2 g/dL, or below 1 g/dL.

In some embodiments, the patient is unable to have a blood transfusion. In certain embodiments, the patient is unable to have a blood transfusion when otherwise clinically indicated due to contraindications. In some embodiments, the patient is unable to have a blood transfusion, wherein the patient has a pre-treatment hemoglobin level below 12 g/dL, or below 10 g/dL. In some embodiments, the patient is unable to have a blood transfusion, wherein the patient has a pre-treatment hemoglobin level below 8 g/dL, below 7 g/dL, below 6 g/dL, below 5 g/dL, below 4 g/dL, below 2 g/dL, or below 1 g/dL.

### 5.3.2. Post-Treatment Endpoints

In the methods described herein, following treatment (post-treatment) with an HIF-PH inhibitor, the patient has a reduction in one or more signs and/or symptoms of an age-related morbidity.

5.3.2.1 Reduction of C-Reactive Protein (CRP)

In some embodiments, the administration of an effective amount of the HIF-PH inhibitor reduces the patient's serum CRP levels below pre-treatment levels.

In some embodiments, the post-treatment CRP level is no more than 45 mg/L. In certain embodiments, the post-treatment CRP level is no more than 40 mg/L. In certain embodiments, the post-treatment CRP level is no more than 30 mg/L. In certain embodiments, the post-treatment CRP level is no more than 20 mg/L. In certain embodiments, the post-treatment CRP level is no more than 10 mg/L. In certain embodiments, the post-treatment CRP level is no more than 5 mg/L. In certain embodiments, the post-treatment CRP level is no more than 2.5 mg/L. In certain embodiments, the post-treatment CRP level is no more than 2 mg/L. In certain embodiments, the post-treatment CRP level is no more than 1 mg/L.

In some embodiments, the CRP level is decreased by at least 10% as compared to pre-treatment levels. In various embodiments, the CRP level is decreased by at least 20%, 30%, 40%, 50%, 60%, 70%, 80%, or 90% as compared to pre-treatment levels. In certain embodiments, the CRP level is decreased by at least 20% as compared to pre-treatment levels. In certain embodiments, the CRP level is decreased by at least 30% as compared to pre-treatment levels. In certain embodiments, the CRP level is decreased by at least 40% as compared to pre-treatment levels. In certain embodiments, the CRP level is decreased by at least 50% as compared to pre-treatment levels. In certain embodiments, the CRP level is decreased by at least 60% as compared to pre-treatment levels. In certain embodiments, the CRP level is decreased by at least 70% as compared to pre-treatment levels. In certain embodiments, the CRP level is decreased by at least 80% as compared to pre-treatment levels. In certain embodiments, the CRP level is decreased by at least 90% as compared to pre-treatment levels.

5.3.2.2 Induction in Hb Levels

In some embodiments, the administration of an effective amount of the HIF-PH inhibitor increases the patient's serum Hb levels above pre-treatment levels.

In some embodiments, the post-treatment Hb level is greater than 10 g/dl. In some embodiments, the post-treatment Hb level is greater than 10.5 g/dl. In certain embodiments, the post-treatment Hb level is greater than 11 g/dl. In some embodiments, the post-treatment Hb level is greater than 11.5 g/dl. In certain embodiments, the post-treatment Hb level is greater than 12 g/dl. In some embodiments, the post-treatment Hb level is greater than 12.5 g/dl. In certain embodiments, the post-treatment Hb level is greater than 13 g/dl. In some embodiments, the post-treatment Hb level is greater than 13.5 g/dl. In certain embodiments, the post-treatment Hb level is greater than 14 g/dl. In some embodiments, the post-treatment Hb level is greater than 14.5 g/dl. In certain embodiments, the post-treatment Hb level is greater than 15 g/dl. In some embodiments, the post-treatment Hb level is greater than 15.5 g/dl. In certain embodiments, the post-treatment Hb level is greater than 16 g/dl. In some embodiments, the post-treatment Hb level is greater than 16.5 g/dl. In certain embodiments, the post-treatment Hb level is greater than 17 g/dl. In some embodiments, the post-treatment Hb level is greater than 17.5 g/dl. In certain embodiments, the post-treatment Hb level is greater than 18 g/dl. In some embodiments, the post-treatment Hb level is greater than 18.5 g/dl. In certain embodiments, the post-treatment Hb level is greater than 19 g/dl. In some embodiments, the post-treatment Hb level is greater than 19.5 g/dl. In certain embodiments, the post-treatment Hb level is greater than 20 g/dl.

In some embodiments, the Hb level is increased by at least 10% as compared to pre-treatment levels. In various embodiments, the Hb level is increased by at least 20%, 30%, 40%, 50%, 60%, 70%, 80%, or 90% as compared to pre-treatment levels. In certain embodiments, the Hb level is increased by at least 20% as compared to pre-treatment levels. In certain embodiments, the Hb level is increased by at least 30% as compared to pre-treatment levels. In certain embodiments, the Hb level is increased by at least 40% as compared to pre-treatment levels. In certain embodiments, the Hb level is increased by at least 50% as compared to pre-treatment levels. In certain embodiments, the Hb level is increased by at least 60% as compared to pre-treatment levels. In certain embodiments, the Hb level is increased by at least 70% as compared to pre-treatment levels. In certain embodiments, the Hb level is increased by at least 80% as compared to pre-treatment levels. In certain embodiments, the Hb level is increased by at least 90% as compared to pre-treatment levels.

### 5.3.3. HIF-Inhibitors of Formula (I′), Formula (I″), Formula (I′-2), and Formula (I)

In some embodiments, the HIF-PH inhibitor is a compound represented by the following general formula (I′):

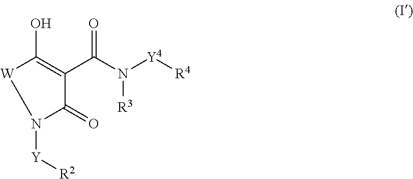

wherein in formula (I′), W represents the formula —CR¹¹R¹²CR¹³R¹⁴;
R¹¹ represents a hydrogen atom, C₁₋₄ alkyl, or phenyl;
R¹² represents a hydrogen atom, a fluoride atom or C₁₋₄ alkyl; or
R¹¹ and R¹², together with the adjacent carbon atom, form a C₃₋₈ cycloalkane or a 4- to 8-membered saturated heterocycle containing an oxygen atom;
R¹³ represents a hydrogen atom, carbamoyl, C₁₋₄ alkyl, wherein the C₁₋₄ alkyl is optionally substituted by one group selected from the group consisting of hydroxy, C₁₋₃ alkoxy, and di-C₁₋₃ alkylamino, halo-C₁₋₄ alkyl, phenyl, pyridyl, benzyl, or phenethyl;
R¹⁴ represents a hydrogen atom, C₁₋₄ alkyl, or halo-C₁₋₄ alkyl; or
R¹³ and R¹⁴, together with the adjacent carbon atom, form a C₃₋₈ cycloalkane, a 4- to 8-membered saturated heterocycle containing an oxygen atom, or a 4- to 8-membered saturated heterocycle containing a nitrogen atom, wherein the 4- to 8-membered saturated heterocycle containing a nitrogen atom is optionally substituted by one or two groups which are the same or different and are selected from the group consisting of methyl, benzyl, phenylcarbonyl, and oxo; or
R¹² and R¹³, together with the adjacent carbon atoms, form a C₃₋₈ cycloalkane;
Y represents a single bond or C₁₋₆ alkanediyl, wherein the C₁₋₆ alkanediyl is optonally substituted by one hydroxyl, and one of the carbon atoms in the C₁₋₆ alkanediyl is optionally substituted by C₃₋₆ cycloalkanel,1-diyl;
R² represents:
a hydrogen atom,
C₁₋₆ akyl,
C₃₋₈ cycloalkyl, wherein the C₃₋₈ cycloalkyl is optionally substituted by one or two groups which are the same or different and are selected from the group consisting of C₁₋₆ alkyl which is optionally substituted by one phenyl, phenyl which is optionally substituted by one group selected from the group consisting of a halogen atom and halo-C₁₋₆ alkyl, C₁₋₆ alkoxy which is optionally substituted by one group selected from the group consisting of C₃₋₈ cycloalkyl, phenyl optionally substituted by one group selected from the group consisting of a halogen atom and C₁₋₆ alkyl, and pyridyl optionally substituted by one halogen atom, C₃₋₈ cycloalkoxy, phenoxy which is optionally substituted by one group selected from the group consisting of a halogen atom, C₁₋₆ alkyl, C₃₋₈ cycloalkyl, and halo-C₁₋₆ alkyl, and pyridyloxy which is optionally substituted by one group selected from the group consisting of a halogen atom, C₁₋₆ alkyl, C₃₋₈ cycloalkyl, and halo-C₁₋₆ alkyl,
phenyl, wherein the phenyl is optionally substituted by one to three groups which are the same or different and are selected from group α3 of substituents,
naphthyl,
indanyl,
tetrahydronaphthyl,
pyrazolyl,
imidazolyl,
isoxazolyl,
oxazolyl, wherein the pyrazolyl, imidazolyl, isoxazolyl, and oxazolyl are optionally substituted by one or two groups which are the same or different and are selected from the group consisting of C₁₋₆ alkyl and phenyl which is optionally substituted by one group selected from the group consisting of a halogen atom and C₁₋₆ alkyl,
thiazoyl, wherein the thiazoyl is optionally substituted by one or two groups which are the same or different and are selected from the group consisting of C₁₋₆ alkyl, phenyl which is optionally substituted by one group selected from the group consisting of a halogen atom and C₁₋₆ alkyl, and morpholino,
pyridyl, wherein the pyridyl is optionally substituted by one or two groups which are the same or different and are selected from group α5 of substituents,
pyridazinyl,
pyrimidinyl,
pyrazinyl,
wherein the pyridazinyl, pyrimidinyl, and pyrazinyl are optionally substituted by one group selected from the group consisting of C₁₋₆ alkyl, halo-C₁₋₆ alkyl, C₃₋₈ cycloalkyl, phenyl, C₁₋₆ alkoxy which is optionally substituted by one C₃₋₈ cycloalkyl, and phenoxy which is optionally substituted by one group selected from the group consisting of a halogen atom, C₁₋₆ alkyl, and C₃₋₈ cycloalkyl,
benzothiophenyl,
quinolyl,
methylenedioxyphenyl, wherein the methylenedioxyphenyl is optionally substituted by one or two fluorine atoms,
4- to 8-membered saturated heterocyclyl containing a nitrogen atom, wherein the 4- to 8-membered saturated heterocyclyl containing a nitrogen atom is optionally substituted by one group selected from the group consisting of pyrimidinyl, phenyl-C₁₋₃ alkyl, C₃₋₈ cycloalkyl-C₁₋₃ alkylcarbonyl, and phenyl-C₁₋₃ alkoxycarbonyl, or

the following formula (I″)
—CONR⁵CH₂—R⁶ (I″), wherein in formula (I″):
   R⁵ represents a hydrogen atom or C₁₋₃ alkyl, and R⁶ represents phenyl which is optionally substituted by one group selected from the group consisting of a halogen atom, C₁₋₆ alkyl, halo-C₁₋₆ alkyl, and phenyl,
   group α3 of substituents consists of:
   hydroxy,
   cyano,
   carboxy,
   a halogen atom,
   C₁₋₆ alkyl, wherein the C₁₋₆ alkyl is optionally substituted by one group selected from the group consisting of C₃₋₈ cycloalkyl, phenyl, C₁₋₆ alkoxy which is optionally substituted by one C₃₋₈ cycloalkyl optionally substituted by one C₁₋₆ alkyl , phenoxy which is optionally substituted by one C₁₋₆ alkyl, and pyridyloxy which is optionally substituted by one group selected from the group consisting of C₁₋₆ alkyl and halo-C₁₋₆ alkyl,
   halo-C₁₋₆ alkyl,
   C₃₋₈ cycloalkyl, wherein the C₃₋₈ cycloalkyl is optionally substituted by one or two halogen atoms,
   C₃₋₈ cycloalkenyl, wherein the C₃₋₈ cycloalkenyl is optionally substituted by one or two halogen atoms,
   phenyl, wherein the phenyl is optionally substituted by one to three groups which are the same or different and are selected from group α4 of substituents,
   thienyl, wherein the thienyl is optionally substituted by one C₁₋₆ alkyl,
   pyrazolyl, wherein the pyrazolyl is optionally substituted by one C₁₋₆ alkyl,
   isoxazolyl,
   thiazoyl, wherein the thiazoyl is optionally substituted by one or two groups which are the same or different and are selected from the group consisting of hydroxy, C₁₋₆ alkyl, and C₁₋₆ alkoxy,
   pyridyl, wherein the pyridyl is optionally substituted by one group selected from the group consisting of carboxy, hydroxy, amino, a halogen atom, C₁₋₆ alkyl, halo-C₁₋₆ alkyl, C₃₋₈ cycloalkyl, C₁₋₆ alkoxy, halo-C₁₋₆ alkoxy, and C₁₋₆ alkylsulfonyl,
   pyrimidinyl, wherein the pyrimidinyl is optionally substituted by one
   amino,
   quinolyl,
   C₁₋₆ alkoxy, wherein the C₁₋₆ alkoxy is optionally substituted by one group selected from the group consisting of carboxy, hydroxy, carbamoyl, C₃₋₈ cycloalkyl which is optionally substituted by one C₁₋₆ alkyl, phenyl which is optionally substituted by one group selected from the group consisting of hydroxy, a halogen atom, C₁₋₆ alkyl, halo-C₁₋₆ alkyl, C₁₋₆ alkoxy, halo-C₁₋₆ alkoxy, and di-C₁₋₆ alkylamino, pyridyl which is optionally substituted by one group selected from the group consisting of a halogen atom and C₁₋₆ alkyl, benzotriazolyl, imidazothiazoyl, di-C₁₋₆ alkylamino, oxazolyl which is optionally substituted by one or two C₁₋₆ alkyls, pyrazolyl, which is optionally substituted by one or two C₁₋₆ alkyls, thiazoyl which is optionally substituted by one C₁₋₆ alkyl, and indazolyl which is optionally substituted by one C₁₋₆ alkyl,
   halo-C₁₋₆ alkoxy,
   C₂₋₆ alkenyloxy,
   C₃₋₈ cycloalkoxy,
   phenoxy, wherein the phenoxy is optionally substituted by one or two groups which are the same or different and are selected from the group consisting of a halogen atom, C₁₋₆ alkyl, halo-C₁₋₆ alkyl, C₁₋₆ alkoxy, and halo-C₁₋₆ alkoxy,
   pyridyloxy, wherein the pyridyloxy is optionally substituted by one group selected from the group consisting of a halogen atom, C₁₋₆ alkyl, halo-C₁₋₆ alkyl, and C₃₋₈ cycloalkyl,
   pyrimidinyloxy,
   piperazinyl, wherein the piperazinyl is optionally substituted by one C₁₋₆ alkyl,
   mono-C₁₋₆ alkylaminocarbonyl, wherein the C₁₋₆ alkyl in the mono-C₁₋₆ alkylaminocarbonyl is optionally substituted by one group selected from the group consisting of carboxy, hydroxy, di-C₁₋₆ alkylamino, pyridyl, phenyl, and 2-oxopyrrolidinyl,
   di-C₁₋₆ alkylaminocarbonyl, wherein the two C₁₋₆ alkyls in the di-C₁₋₆ alkylaminocarbonyl, together with the adjacent nitrogen atom, optionally form a 4- to 8-membered saturated heterocycle containing a nitrogen atom,
   C₁₋₆ alkylsulfanyl, and
   C₁₋₆ alkylsulfonyl;

group α4 of substituents consists of:
carboxy,
   cyano,
   hydroxy,
   sulfamoyl,
   a halogen atom,
   C₁₋₆ alkyl,
   halo-C₁₋₆ alkyl,
   C₃₋₈ cycloalkyl,
   phenyl,
   C_{1.6} alkoxy,
   halo-C₁₋₆ alkoxy,
   C₁₋₆ alkylcarbonyl,
   di-C₁₋₆ alkylaminocarbonyl,
   C₁₋₆ alkylsulfonyl,
   mono-C₁₋₆ alkylaminosulfonyl, wherein the C₁₋₆ alkyl in the mono-C₁₋₆ alkylaminosulfonyl is optionally substituted by one hydroxy, and
   di-C₁₋₆ alkylaminosulfonyl;
group α5 of substituents consist of:
   a halogen atom,
   C₁₋₆ alkyl,
   halo-C₁₋₆ alkyl,
   C₁₋₆ alkoxy, wherein the C₁₋₆ alkoxy is optionally substituted by one group selected from the group consisting of C₃₋₈ cycloalkyl which is optionally substituted by one C₁₋₆ alkyl and phenyl which is optionally substituted by one group selected from the group consisting of a halogen atom and C₁₋₆ alkyl,
   halo-C₁₋₆ alkoxy,
   phenyl, wherein the phenyl is optionally substituted by one group selected from group α6 of substituents,
   pyridyl,
   phenoxy, wherein the phenoxy is optionally substituted by one or two groups which are the same or different and are selected from the group consisting of a halogen atom, cyano, C₁₋₆ alkyl, halo-C₁₋₆ alkyl, C₃₋₈ cycloalkyl, halo-C₁₋₆ alkoxy and C₁₋₆ alkoxy which is optionally substituted by one phenyl, and,
   pyridyloxy, wherein the pyridyloxy is optionally substituted by one C₁₋₆ alkyl, and
   phenylsulfanyl, wherein the phenylsulfanyl is optionally substituted by one halogen atom;
group α6 of substituents consists of:
   a halogen atom,
   C₁₋₆ alkyl,
   halo-C₁₋₆ alkyl,
   C₃₋₈ cycloalkyl,
   C₁₋₆ alkoxy, and
   halo-C₁₋₆ alkoxy;
   Y⁴ represents C₁₋₄ alkanediyl;
   R³ represents a hydrogen atom or methyl;
   R⁴ represents —COOH, —CONHOH, or tetrazolyl;
   or a pharmaceutically acceptable salt thereof.

In certain embodiments, in the compound in formula (I′):
Y⁴ is methanediyl,
R³ is a hydrogen atom,
R⁴ is —COOH, or a pharmaceutically acceptable salt thereof.

In certain embodiments, the compound is represented by general formula (I′-2):

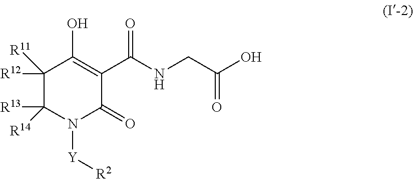

wherein in formula (I′-2):
R¹¹ is a hydrogen atom, a fluorine atom, C₁₋₄ alkyl, or phenyl,
R¹² is a hydrogen atom, a fluorine atom, or C₁₋₄ alkyl, or
R¹¹ and R¹², together with the adjacent carbon atom, form a C₃₋₈ cycloalkane or a 4- to 8-membered saturated heterocycle containing an oxygen atom;
R¹³ is a hydrogen atom, carbamoyl, C₁₋₄ alkyl, wherein the C₁₋₄ alkyl is optionally substituted by one group selected from the group consisting of hydroxy, C₁₋₃ alkoxy, and di-C₁₋₃ alkylamino, halo-C₁₋₄ alkyl, phenyl, pyridyl, benzyl, or phenethyl;
R¹⁴ is a hydrogen atom, C₁₋₄ alkyl, or halo-C₁₋₄ alkyl, or
R¹³ and R¹⁴, together with the adjacent carbon atom, form a C₃₋₈ cycloalkane, a 4- to 8-membered saturated heterocycle containing an oxygen atom, or a 4- to 8-membered saturated heterocycle containing a nitrogen atom, wherein the 4- to 8-membered saturated heterocycle containing a nitrogen atom is optionally substituted by one or two groups which are the same or different and are selected from the group consisting of methyl, benzyl, phenylcarbonyl, and oxo, or
R¹² and R¹³, together with the adjacent carbon atoms, form a C₃₋₈ cycloalkane,
or a pharmaceutically acceptable salt thereof.

In certain embodiments, in the compound of formula (I′-2):
Y is a single bond or C₁₋₆ alkanediyl, wherein one of the carbon atoms in the C₁₋₆ alkanediyl is optionally substituted by C₃₋₆ cycloalkane-1,1-diyl,
R² is:
   C₃₋₈ cycloalkyl, wherein the C₃₋₈ cycloalkyl is optionally substituted by one or two groups which are the same or different and are selected from the group consisting of C₁₋₆ alkyl which is optionally substituted by one phenyl, phenyl which is optionally substituted by one halo-C₁₋₆ alkyl, C₁₋₆ alkoxy which is optionally substituted by one group selected from the group consisting of C₃₋₈ cycloalkyl, phenyl optionally substituted by one group selected from the group consisting of a halogen atom and C₁₋₆ alkyl, and pyridyl optionally substituted by one halogen atom, C₃₋₈ cycloalkoxy, phenoxy which is optionally substituted by one group selected from the group consisting of a halogen atom, C₁₋₆ alkyl, C₃₋₈ cycloalkyl, and halo-C₁₋₆ alkyl, and pyridyloxy which is optionally substituted by one group selected from the group consisting of a halogen atom, C₁₋₆ alkyl, C₃₋₈ cycloalkyl, and halo-C₁₋₆ alkyl,
phenyl, wherein the phenyl is optionally substituted by one to three groups which are the same or different and are selected from the aforementioned group α3 of substituents,
   naphthyl,
   indanyl,
   tetrahydronaphthyl,
   pyrazolyl, wherein the pyrazolyl is optionally substituted by one or two groups which are the same or different and are selected from the group consisting of C₁₋₆ alkyl and phenyl which is optionally substituted by one C₁₋₆ alkyl,
   imidazolyl, wherein the imidazolyl is optionally substituted by one group selected from the group consisting of C₁₋₆ alkyl and phenyl,
   isoxazolyl, wherein the isoxazolyl is optionally substituted by one phenyl which is optionally substituted by one halogen atom,
   oxazolyl, wherein the oxazolyl is optionally substituted by one or two groups which are the same or different and are selected from the group consisting of C₁₋₆ alkyl and phenyl,
   thiazoyl, wherein the thiazoyl is optionally substituted by one group selected from the group consisting of C₁₋₆ alkyl, phenyl, and morpholino,
   pyridyl, wherein the pyridyl is optionally substituted by one or two groups which are the same or different and are selected from the aforementioned group α5 of substituents,
   pyridazinyl, wherein the pyridazinyl is optionally substituted by one C₁₋₆ alkoxy which is optionally substituted by one C₃₋₈ cycloalkyl,
   pyrimidinyl, wherein the pyrimidinyl is optionally substituted by one group selected from the group consisting of halo-C₁₋₆ alkyl, C₃₋₈ cycloalkyl, phenyl, and phenoxy which is optionally substituted by one C₁₋₆ alkyl,
   pyrazinyl, wherein the pyrazinyl is optionally substituted by one group selected from the group consisting of C₁₋₆ alkoxy which is optionally substituted by one C₃₋₈ cycloalkyl and phenoxy which is optionally substituted by one group selected from the group consisting of a halogen atom, C₁₋₆ alkyl, and C₃₋₈ cycloalkyl,
   benzothiophenyl,
   quinolyl, or
   methylenedioxyphenyl, wherein the methylenedioxyphenyl is optionally substituted by one or two fluorine atoms,
   or a pharmaceutically acceptable salt thereof.

In certain embodiments, in the compound of formula (I′-2):
R¹¹ is a hydrogen atom,
R¹² is a hydrogen atom,
R¹³ is a hydrogen atom,
R¹⁴ is a hydrogen atom,
Y is methanediyl,
R² is:
   phenyl, wherein the phenyl is substituted by one group selected from the group consisting of phenyl which is optionally substituted by one or two groups which are the same or different and are selected from the group consisting of carboxy, cyano, hydroxy, sulfamoyl, a halogen atom, C₁₋₆ alkyl, halo-C₁₋₆ alkyl, C₃₋₈ cycloalkyl, phenyl, C₁₋₆ alkoxy, halo-C₁₋₆ alkoxy, C₁₋₆ alkylcarbonyl, di-C₁₋₆ alkylaminocarbonyl, C₁₋₆ alkyl sulfonyl, di-C₁₋₆ alkylaminosulfonyl and mono-C₁₋₆ alkylaminosulfonyl, wherein the C₁₋₆ alkyl in the mono-C₁₋₆alkylaminosulfonyl is optionally substituted by one hydroxy, pyridyl which is optionally substituted by one group selected from the group consisting of carboxy, hydroxy, amino, a halogen atom, C₁₋₆ alkyl, halo-C₁₋₆ alkyl, C₃₋₈ cycloalkyl, C₁₋₆ alkoxy, and C₁₋₆ alkylsulfonyl, phenoxy which is optionally substituted by one or two groups which are the same or different and are selected from the group consisting of a halogen atom, C₁₋₆ alkyl, C₁₋₆ alkoxy, and halo-C₁₋₆ alkoxy, and pyridyloxy which is optionally substituted by one group selected from the group consisting of a halogen atom, C₁₋₆ alkyl, halo-C₁₋₆ alkyl, and C₃₋₈ cycloalkyl, and said substituted phenyl represented by R² may further be substituted by one halogen atom;
   pyridyl, wherein the pyridyl is substituted by one group selected from the group consisting of phenyl which is optionally substituted by one group selected from the group consisting of a halogen atom, C₁₋₆ alkyl, halo-C₁₋₆ alkyl, C₃₋₈ cycloalkyl, C₁₋₆ alkoxy, and halo-C₁₋₆ alkoxy, pyridyl, phenoxy which is optionally substituted by one or two groups which are the same or different and are selected from the group consisting of a halogen atom, cyano, C₁₋₆ alkyl, halo-C₁₋₆ alkyl, C₃₋₈ cycloalkyl, halo-C₁₋₆ alkoxy and C₁₋₆ alkoxy optionally substituted by one phenyl, and pyridyloxy which is optionally substituted by one C₁₋₆ alkyl, and said substituted pyridyl represented by R² may further be substituted by one group selected from the group consisting of a halogen atom and C₁₋₆ alkyl; or
   pyrazinyl which is substituted by one phenoxy wherein the phenoxy is optionally substituted by one group selected from the group consisting of a halogen atom, C₁₋₆ alkyl, and C₃₋₈ cycloalkyl,
   or a pharmaceutically acceptable salt thereof.

In a particular embodiment, the compound is N-{[4-hydroxy-2-oxo-1-(4-phenoxybenzyl)-1,2,5,6-tetrahydro-3-pyridinyl]carbonyl}glycine, or a pharmaceutically acceptable salt thereof.

In a particular embodiment, the compound is N-[(4-hydroxy-1-{1[6-(4-methylphenoxy)-3-pyridinyl]methyl}-2-oxo-1,2,5,6-tetrahydro-3-pyridinyl)carbonyl]glycine, or a pharmaceutically acceptable salt thereof.

In a particular embodiment, the compound is N-({4-hydroxy-2-oxo-1-[(6-phenoxy-3-pyridinyl)methyl]-1,2,5,6-tetrahydro-3-pyridinyl}carbonyl)glycine, or a pharmaceutically acceptable salt thereof.

In a particular embodiment, the compound is N-({1-[4-(4-fluorophenoxy)benzyl]-4-hydroxy-2-oxo-1,2,5,6-tetrahydro-3-pyridinyl}carbonyl)glycine, or a pharmaceutically acceptable salt thereof.

In a particular embodiment, the compound is N-({4-hydroxy-1-[4-(4-methylphenoxy)benzyl]-2-oxo-1,2,5,6-tetrahydro-3-pyridinyl}carbonyl)glycine, or a pharmaceutically acceptable salt thereof.

In a particular embodiment, the compound is N-[(1-{[6-(4-cyanophenoxy)-3-pyridinyl]methyl}-4-hydroxy-2-oxo-1,2,5,6-tetrahydro-3-pyridinyl)carbonyl]glycine;

In a particular embodiment, the compound is N-({4-hydroxy-2-oxo-1-[4-(2-pyrimidinyloxy)benzyl]-1,2,5,6-tetrahydro-3-pyridinyl}carbonyl)glycine, or a pharmaceutically acceptable salt thereof.

In a particular embodiment, the compound is N-[(1-{[6-(4-fluorophenoxy)-3-pyridinyl]methyl}-4-hydroxy-2-oxo-1,2,5,6-tetrahydro-3-pyridinyl)carbonyl]glycine;

In a particular embodiment, the compound is N-[(1{[-6-(4-chlorophenoxy)-3-pyridinyl]methyl}-4-hydroxy-2-oxo-1,2,5,6-tetrahydro-3-pyridinyl)carbonyl]glycine, or a pharmaceutically acceptable salt thereof.

In a particular embodiment, the compound is N-{[4-hydroxy-2-oxo-1-({6-[4-(trifluoromethyl)phenoxy]-3-pyridinyl}methyl)-1,2,5,6-tetrahydro-3-pyridinyl]carbonyl}glycine, or a pharmaceutically acceptable salt thereof.

In a particular embodiment, the compound is N-[(4-hydroxy-1-{[6-(3-methylphenoxy)-3-pyridinyl]methyl}-2-oxo-1,2,5,6-tetrahydro-3-pyridinyl)carbonyl]glycine, or a pharmaceutically acceptable salt thereof.

In a particular embodiment, the compound is N-[(1-{[6-(3-fluorophenoxy)-3-pyridinyl]methyl}-4-hydroxy-2-oxo-1,2,5,6-tetrahydro-3-pyridinyl)carbonyl]glycine, or a pharmaceutically acceptable salt thereof.

In a particular embodiment, the compound is N-({4-hydroxy-1-[4-(3-methylphenoxy)benzyl]-2-oxo-1,2,5,6-tetrahydro-3-pyridinyl}carbonyl)glycine, or a pharmaceutically acceptable salt thereof.

In a particular embodiment, the compound is N-({1-[4-(3-fluorophenoxy)benzyl]-4-hydroxy-2-oxo-1,2,5,6-tetrahydro-3-pyridinyl}carbonyl)glycine;

In a particular embodiment, the compound is N-[1-{[5-(4-fluorophenoxy)-2-pyridinyl]methyl}-4-hydroxy-2-oxo-1,2,5,6-tetrahydro-3-pyridinyl)carbonyl]glycine, or a pharmaceutically acceptable salt thereof.

In a particular embodiment, the compound is N-[(4-hydroxy-1-{[5-(4-methylphenoxy)-2-pyridinyl]methyl}-2-oxo-1,2,5,6-tetrahydro-3-pyridinyl)carbonyl]glycine, or a pharmaceutically acceptable salt thereof.

In a particular embodiment, the compound is N-({1-[4-(4-chlorophenoxy)benzyl]-4-hydroxy-2-oxo-1,2,5,6-tetrahydro-3-pyridinyl}carbonyl)glycine, or a pharmaceutically acceptable salt thereof.

In a particular embodiment, the compound is N-[(4-hydroxy-1-{4-[(6-methyl-3-pyridinyl)oxy]benzyl}-2-oxo-1,2,5,6-tetrahydro-3-pyridinyl)carbonyl]glycine, or a pharmaceutically acceptable salt thereof.

In a particular embodiment, the compound is N-[(1-{[6-(2-fluorophenoxy)-3-pyridinyl]methyl}-4-hydroxy-2-oxo-1,2,5,6-tetrahydro-3-pyridinyl)carbonyl]glycine;

In a particular embodiment, the compound is N-[(4-hydroxy-1-{[6-(2-methylphenoxy)-3-pyridinyl]methyl}-2-oxo-1,2,5,6-tetrahydro-3-pyridinyl)carbonyl]glycine, or a pharmaceutically acceptable salt thereof.

In a particular embodiment, the compound is N-({1-[4-(2-fluorophenoxy)benzyl]-4-hydroxy-2-oxo-1,2,5,6-tetrahydro-3-pyridinyl}carbonyl)glycine, or a pharmaceutically acceptable salt thereof.

In a particular embodiment, the compound is N-({4-hydroxy-1-[4-(2-methylphenoxy)benzyl]-2-oxo-1,2,5,6-tetrahydro-3-pyridinyl}carbonyl)glycine, or a pharmaceutically acceptable salt thereof.

In a particular embodiment, the compound is N-[(1-{[6-(3-chlorophenoxy)-3-pyridinyl]methyl}-4-hydroxy-2-oxo-1,2,5,6-tetrahydro-3-pyridinyl)carbonyl]glycine, or a pharmaceutically acceptable salt thereof.

In a particular embodiment, the compound is N-{[4-hydroxy-2-oxo-1-({6-[3-(trifluoromethyl)phenoxy]-3-pyridinyl}methyl)-1,2,5,6-tetrahydro-3-pyridinyl]carbonyl}glycine, or a pharmaceutically acceptable salt thereof.

In a particular embodiment, the compound is N-({4-hydroxy-1-[4-(3-methoxyphenoxy)benzyl]-2-oxo-1,2,5,6-tetrahydro-3-pyridinyl}carbonyl)glycine, or a pharmaceutically acceptable salt thereof.

In a particular embodiment, the compound is N-{[4-hydroxy-2-oxo-1-({6-[3-(trifluoromethoxy)phenoxy]-3-pyridinyl}methyl)-1,2,5,6-tetrahydro-3-pyridinyl]carbonyl}glycine, or a pharmaceutically acceptable salt thereof.

In a particular embodiment, the compound is N-[(1-{4-[(5-fluoro-2-pyridinyl)oxy]benzyl}-4-hydroxy-2-oxo-1,2,5,6-tetrahydro-3-pyridinyl)carbonyl]glycine, or a pharmaceutically acceptable salt thereof.

In a particular embodiment, the compound is N-[(1-{4-[(5-chloro-2-pyridinyl)oxy]benzyl}-4-hydroxy-2-oxo-1,2,5,6-tetrahydro-3-pyridinyl)carbonyl]glycine, or a pharmaceutically acceptable salt thereof.

In a particular embodiment, the compound is N-[1-{[(6-(4-cyclopropylphenoxy)-3-pyridinyl]methyl}-4-hydroxy-2-oxo-1,2,5,6-tetrahydro-3-pyridinyl)carbonyl]glycine, or a pharmaceutically acceptable salt thereof.

In a particular embodiment, the compound is N-[(4-hydroxy-1-{4-[(5-methyl-2-pyridinyl)oxy]benzyl}-2-oxo-1,2,5,6-tetrahydro-3-pyridinyl)carbonyl]glycine, or a pharmaceutically acceptable salt thereof.

In a particular embodiment, the compound is N-{[4-hydroxy-2-oxo-1-(4-{[5-(trifluoromethyl)-2-pyridinyl]oxy}benzyl)-1,2,5,6-tetrahydro-3-pyridinyl]carbonyl}glycine, or a pharmaceutically acceptable salt thereof.

In a particular embodiment, the compound is N-{[4-hydroxy-1-({5-methyl-6-[(6-methyl-3-pyridinyl)oxy]-3-pyridinyl}methyl)-2-oxo-1,2,5,6-tetrahydro-3-pyridinyl]carbonyl}glycine, or a pharmaceutically acceptable salt thereof.

In a particular embodiment, the compound is N-[(1-{[5-(4-chlorophenoxy)-2-pyridinyl]methyl}-4-hydroxy-2-oxo-1,2,5,6-tetrahydro-3-pyridinyl)carbonyl]glycine, or a pharmaceutically acceptable salt thereof.

In a particular embodiment, the compound is N-[(4-hydroxy-1-{[6-(3-methoxyphenoxy)-3-pyridinyl]methyl}-2-oxo-1,2,5,6-tetrahydro-3-pyridinyl)carbonyl]glycine;

In a particular embodiment, the compound is N-[(1-{4-[(6-chloro-3-pyridinyl)oxy]benzyl}-4-hydroxy-2-oxo-1,2,5,6-tetrahydro-3-pyridinyl)carbonyl]glycine, or a pharmaceutically acceptable salt thereof.

In a particular embodiment, the compound is N-{[4-hydroxy-2-oxo-1-({5-[4-(trifluoromethyl)phenoxy]-2-pyridinyl}methyl)-1,2,5,6-tetrahydro-3-pyridinyl]carbonyl}glycine;

In a particular embodiment, the compound is N-{[4-hydroxy-2-oxo-1-(4-{[6-(trifluoromethyl)-3-pyridinyl]oxy}benzyl)-1,2,5,6-tetrahydro-3-pyridinyl]carbonyl}glycine, or a pharmaceutically acceptable salt thereof.

In a particular embodiment, the compound is N-[(1-{[6-(3-chloro-4-methylphenoxy)-3-pyridinyl]methyl}-4-hydroxy-2-oxo-1,2,5,6-tetrahydro-3-pyridinyl)carbonyl]glycine, or a pharmaceutically acceptable salt thereof.

In a particular embodiment, the compound is N-[(1-{[6-(3-fluoro-4-methylphenoxy)-3-pyridinyl]methyl}-4-hydroxy-2-oxo-1,2,5,6-tetrahydro-3-pyridinyl)carbonyl]glycine, or a pharmaceutically acceptable salt thereof.

In a particular embodiment, the compound is N-[(1-{[6-(4-fluoro-3-methylphenoxy)-3-pyridinyl]methyl}-4-hydroxy-2-oxo-1,2,5,6-tetrahydro-3-pyridinyl)carbonyl]glycine, or a pharmaceutically acceptable salt thereof.

In a particular embodiment, the compound is N-[(1-{[6-(4-ethylphenoxy)-3-pyridinyl]methyl}-4-hydroxy-2-oxo-1,2,5,6-tetrahydro-3-pyridinyl)carbonyl]glycine, or a pharmaceutically acceptable salt thereof.

In a particular embodiment, the compound is N-[(4-hydroxy-2-oxo-1-{[6-(4-propylphenoxy)-3-pyridinyl]methyl}-1,2,5,6-tetrahydro-3pyridinyl)carbonyl]glycine;

In a particular embodiment, the compound is N-[(4-hydroxy-1-{[6-(4-isopropylphenoxy)-3-pyridinyl]methyl}-2-oxo-1,2,5,6-tetrahydro-3-pyridinyl)carbonyl]glycine, or a pharmaceutically acceptable salt thereof.

In a particular embodiment, the compound is N-[(4-hydroxy-1-{[5-(4-methylphenoxy)-2-pyrazinyl]methyl}-2-oxo-1,2,5,6-tetrahydro-3-pyridinyl)carbonyl]glycine, or a pharmaceutically acceptable salt thereof.

In a particular embodiment, the compound is N-({1-[4-(3,4-dimethylphenoxy)benzyl]-4-hydroxy-2-oxo-1,2,5,6-tetrahydro-3-pyridinyl}carbonyl)glycine, or a pharmaceutically acceptable salt thereof.

In a particular embodiment, the compound is N-[(1-{[5-chloro-6-(4-methylphenoxy)-3-pyridinyl]methyl}-4-hydroxy-2-oxo-1,2,5,6-tetrahydro-3-pyridinyl)carbonyl]glycine, or a pharmaceutically acceptable salt thereof.

In a particular embodiment, the compound is N-[(1-{[5-fluoro-6-(4-methylphenoxy)-3-pyridinyl]methyl}-4-hydroxy-2-oxo-1,2,5,6-tetrahydro-3-pyridinyl)carbonyl]glycine, or a pharmaceutically acceptable salt thereof.

In a particular embodiment, the compound is N-[(1-{4-[(5-cyclopropyl-2-pyridinyl)oxy]benzyl}-4-hydroxy-2-oxo-1,2,5,6-tetrahydro-3-pyridinyl)carbonyl]glycine, or a pharmaceutically acceptable salt thereof.

In a particular embodiment, the compound is N-[(4-hydroxy-1-{[2-(4-methylphenoxy)-5-pyrimidinyl]methyl}-2-oxo-1,2,5,6-tetrahydro-3-pyridinyl)carbonyl]glycine; In a particular embodiment, the compound is N-[(1-{[6-(4-chlorophenoxy)-5-methyl-3-pyridinyl]methyl}-4-hydroxy-2-oxo-1,2,5,6-tetrahydro-3-pyridinyl)carbonyl]glycine, or a pharmaceutically acceptable salt thereof.

In a particular embodiment, the compound is N-[(1-{[5-(4-chlorophenoxy)-2-pyrazinyl]methyl}-4-hydroxy-2-oxo-1,2,5,6-tetrahydro-3-pyridinyl)carbonyl]glycine, or a pharmaceutically acceptable salt thereof.

In a particular embodiment, the compound is N-[(1-{[5-(4-cyclopropylphenoxy)-2-pyrazinyl]methyl}-4-hydroxy-2-oxo-1,2,5,6-tetrahydro-3-pyridinyl)carbonyl]glycine, or a pharmaceutically acceptable salt thereof.

In certain embodiments, the compound is represented by general formula (I):

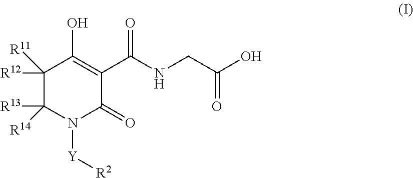

wherein in formula (I):
R¹¹ is a hydrogen atom, C₁₋₄ alkyl, or phenyl,
R¹² is a hydrogen atom or C₁₋₄ alkyl, or
R¹¹ and R¹², together with the adjacent carbon atom, form a C₃₋₈ cycloalkane or a 4- to 8-membered saturated heterocycle containing an oxygen atom;
R¹³ is a hydrogen atom, C₁₋₄ alkyl, halo-C₁₋₄ alkyl, phenyl, benzyl, or phenethyl,
R¹⁴ is a hydrogen atom or C₁₋₄ alkyl, or
R¹³and R¹⁴, together with the adjacent carbon atom, form a C₃₋₈ cycloalkane or a 4- to 8-membered saturated heterocycle containing an oxygen atom, or
R¹² and R¹³, together with the adjacent carbon atoms, form a C₃₋₈ cycloalkane;
Y is a single bond or C₁₋₆ alkanediyl, wherein one of the carbon atoms in the C₁₋₆ alkanediyl is optionally substituted by C₃₋₆ cycloalkane-1,1-diyl;
R² is:
   C₃₋₈ cycloalkyl, wherein the C₃₋₈ cycloalkyl is optionally substituted by one group selected from the group consisting of phenyl and benzyl,
   phenyl, wherein the phenyl is optionally substituted by one to three groups which are the same or different and are selected from group α1 of substituents,
      naphthyl,
      indanyl,
      tetrahydronaphthyl,
      pyrazolyl, wherein the pyrazolyl is substituted by one phenyl, which is optionally substituted by one C₁₋₆ alkyl and may further be substituted by one C₁₋₆ alkyl,
      imidazolyl, wherein the imidazolyl is substituted by one phenyl,
      isoxazolyl, wherein the isoxazolyl is substituted by one phenyl which is optionally substituted by one halogen atom,
      oxazolyl, wherein the oxazolyl is substituted by one phenyl and may further be substituted by one C₁₋₆ alkyl,
      thiazoyl, wherein the thiazoyl is substituted by one phenyl,
      pyridyl, wherein the pyridyl is substituted by one group selected from the group consisting of phenyl, phenoxy which is optionally substituted by one group selected from the group consisting of a halogen atom, cyano, C₁₋₆ alkyl, halo-C₁₋₆ alkyl, C₃₋₈ cycloalkyl, C₁₋₆ alkoxy, and halo-C₁₋₆ alkoxy), and phenylsulfanyl which is optionally substituted by one halogen atom,
      pyrimidinyl, wherein the pyrimidinyl is substituted by one group selected from the group consisting of cyclohexyl and phenyl,
      benzothiophenyl,
      quinolyl, or
      methylenedioxyphenyl, wherein the methylenedioxyphenyl is optionally
      substituted by one or two fluorine atoms;
group α1 of substituents consists of:
   a halogen atom,
   C₁₋₆ alkyl, wherein the C₁₋₆ alkyl is optionally substituted by one group selected from the group consisting of C₃₋₈ cycloalkyl, phenyl, and C₁₋₆ alkoxy which is optionally substituted by one C₃₋₈ cycloalkyl optionally substituted by one C₁₋₆ alkyl,
   halo-C₁₋₆ alkyl,
   C₃₋₈ cycloalkyl,
   phenyl, wherein the phenyl is optionally substituted by one to three groups which are the same or different and are selected from group α2 of substituents,
   thienyl,
   pyrazolyl, wherein the pyrazolyl is optionally substituted by one C₁₋₆ alkyl,
   isoxazolyl,
   thiazoyl, wherein the thiazoyl is optionally substituted by one or two C₁₋₆ alkyls,
   pyridyl, wherein the pyridyl is optionally substituted by one group selected from the group consisting of C₁₋₆ alkyl, halo-C₁₋₆ alkyl, C₁₋₆ alkoxy, and halo-C₁₋₆ alkoxy,
   quinolyl,
   C₁₋₆ alkoxy, wherein the C₁₋₆ alkoxy is optionally substituted by one group selected from the group consisting of C₃₋₈ cycloalkyl and phenyl which is optionally substituted by one group selected from the group consisting of a halogen atom and C₁₋₆ alkyl,
   halo-C₁₋₆ alkoxy,
   C₂₋₆ alkenyloxy,
   C₃₋₈ cycloalkoxy,
   phenoxy, wherein the phenoxy is optionally substituted by one group selected from the group consisting of a halogen atom, C₁₋₆ alkyl, halo-C₁₋₆ alkyl, C₁₋₆ alkoxy, and halo-C₁₋₆ alkoxy,
   pyridyloxy, wherein the pyridyloxy is optionally substituted by one group selected from the group consisting of a halogen atom, C₁₋₆ alkyl, and halo-C₁₋₆ alkyl, and
   C₁₋₆alkylsulfanyl;
group α2 of substituents consists of a halogen atom, cyano, hydroxy, C₁₋₆ alkyl, halo-C₁₋₆ alkyl, phenyl, C₁₋₆ alkoxy, halo-C₁₋₆ alkoxy, C₁₋₆ alkylcarbonyl, and di-C₁₋₆ alkylaminosulfonyl, or a pharmaceutically acceptable salt thereof.

In a particular embodiment, the compound is N-[(1{[6-(4-chlorophenoxy)-3-pyridinyl]methyl}-4-hydroxy-2-oxo-1,2,5,6-tetrahydro-3-pyridinyl)carbonyl]glycine, or a pharmaceutically acceptable salt thereof. The compound “N-[(1{[6-(4-chlorophenoxy)-3-pyridinyl]methyl}-4-hydroxy-2-oxo-1,2,5,6-tetrahydro-3-pyridinyl)carbonyl]glycine”, as used herein, is used interchangeably with “BGE-117” and “TP-518”.

In a particular embodiment, the compound is N-[(1-{[6-(4-cyclopropylphenoxy)-3-pyridinyl]methyl}-4-hydroxy-2-oxo-1,2,5,6-tetrahydro-3-pyridinyl)carbonyl]glycine, or a pharmaceutically acceptable salt thereof.

In a particular embodiment, the compound is N-[(4-hydroxy-1-{[6-(3-methylphenoxy)-3-pyridinyl]methyl}-2-oxo-1,2,5,6-tetrahydro-3-pyridinyl)carbonyl]glycine, or a pharmaceutically acceptable salt thereof.

In a particular embodiment, the compound is N-[(1-{[6-(3-fluorophenoxy)-3-pyridinyl]methyl}-4-hydroxy-2-oxo-1,2,5,6-tetrahydro-3-pyridinyl)carbonyl]glycine, or a pharmaceutically acceptable salt thereof.

In a particular embodiment, the compound is N-[(4-hydroxy-1-{4-[(6-methyl-3-pyridinyl)oxy]benzyl}-2-oxo-1,2,5,6-tetrahydro-3-pyridinyl)carbonyl]glycine, or a pharmaceutically acceptable salt thereof.

In a particular embodiment, the HIF-PH inhibitor is Desidustat, or a pharmaceutically acceptable salt thereof.

In a particular embodiment, the HIF-PH inhibitor is Enarodustat, or a pharmaceutically acceptable salt thereof.

In a particular embodiment, the HIF-PH inhibitor is Molidustat, or a pharmaceutically acceptable salt thereof.

In a particular embodiment, the HIF-PH inhibitor is Roxadustat, or a pharmaceutically acceptable salt thereof.

In a particular embodiment, the HIF-PH inhibitor is Daprodustat, or a pharmaceutically acceptable salt thereof.

In a particular embodiment, the HIF-PH inhibitor is Vadadustat, or a pharmaceutically acceptable salt thereof.

In a particular embodiment, the HIF-PH inhibitor is 1-(6-(2,6-dimethylphenoxy)-7-fluoro-4-oxo-3,4-dihydroquinazolin-2-yl)-1H-pyrazole-4-carboxylic acid (JNJ-42905343).

In a particular embodiment, the HIF-PH inhibitor is JNJ-42041935.

In some embodiments, the dose of HIF-PH inhibitor is at least 0.5 mg/kg PO per day.

In some embodiments, the dose of HIF-PH inhibitor is at least 2 mg/kg PO per day.

In some embodiments, the dose of HIF-PH inhibitor is at least 4 mg/kg PO per day.

In some embodiments, wherein the dose of HIF-PH inhibitor is at least 8 mg/kg PO per day.

In some embodiments, wherein the dose of HIF-PH inhibitor is at least 12 mg/kg PO per day.

In some embodiments, wherein the dose of HIF-PH inhibitor is at least 14 mg/kg PO per day.

In some embodiments, wherein the dose of HIF-PH inhibitor is at least 16 mg/kg PO QD.

In some embodiments, the dose is 0.5 mg/kg.

In some embodiments, the dose is 1 mg/kg.

In some embodiments, the dose is 2 mg/kg.

In some embodiments, the dose is 2.5 to 160 mg/kg.

In some embodiments, the dose is 1 to 30 mg.

In some embodiments, the HIF-PH inhibitor is administered orally.

In some embodiments, the dose is administered daily.

In some embodiments, the dose is administered as a plurality of equally or unequally divided sub-doses.

In some embodiments, the compound is the compound of Formula (3):

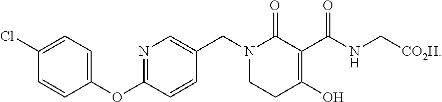 [SMILES: O=C(O)CNC(=O)C1=C(O)CCN(Cc2ccc(Oc3ccc(Cl)cc3)nc2)C1=O]

### 5.3.4. Other HIF-PH Inhibitors

In some embodiments, the HIF-PH inhibitor is a known HIF-PH inhibitor.

In some embodiments, the HIF-PH inhibitor is Enarodustat, or a pharmaceutically acceptable salt thereof. In certain embodiments, the HIF-PH inhibitor is a derivative of Enarodustat.

In some embodiments, the HIF-PH inhibitor is Molidustat, or a pharmaceutically acceptable salt thereof. In certain embodiments, the HIF-PH inhibitor is a derivative of Molidustat.

In some embodiments, the HIF-PH inhibitor is Roxadustat, or a pharmaceutically acceptable salt thereof. In certain embodiments, the HIF-PH inhibitor is a derivative of Roxadustat.

In some embodiments, the HIF-PH inhibitor is Daprodustat, or a pharmaceutically acceptable salt thereof. In certain embodiments, the HIF-PH inhibitor is a derivative of Daprodustat.

In some embodiments, the HIF-PH inhibitor is Vadadustat, or a pharmaceutically acceptable salt thereof. In certain embodiments, the HIF-PH inhibitor is a derivative of Vadadustat.

In some embodiments, the HIF-PH inhibitor is Desidustat, or a pharmaceutically acceptable salt thereof. In certain embodiments, the HIF-PH inhibitor is a derivative of Desidustat.

In some embodiments, the HIF-PH inhibitor is dimethyloxalylglycine.

In some embodiments, the HIF-PH inhibitor is IOX2, having the compound of Formula (IV):

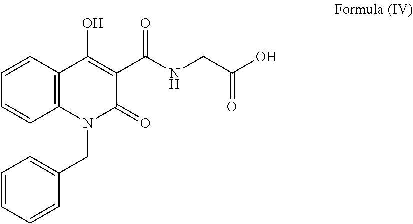 [SMILES: O=C(O)CNC(=O)c1c(O)c2ccccc2n(Cc2ccccc2)c1=O]

In some embodiments, the compound is (N-[[1,2-Dihydro-4-hydroxy-2-oxo-1-(phenylmethyl)-3-quinolinyl]carbonyl]-glycine, N-[[4-Hydroxy-2-oxo-1-(phenylmethyl)-1,2-dihydro-3-quinolinyl]carbonyl]glycine).

In some embodiments, the HIF-PH inhibitor is IOX3, having the compound of formula (V):

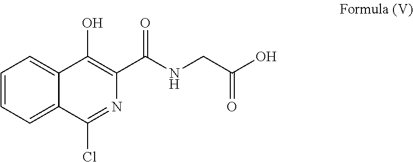 [SMILES: O=C(O)CNC(=O)c1nc(Cl)c2ccccc2c1O]

In some embodiments, the compound is N-[(1-Chloro-4-hydroxy-3-isoquinolinyl)carbonyl]glycine.

In some embodiments, the HIF-PH inhibitor is a hypoxia-inducible factor-1 alpha (HIF-1α) prolyl hydroxylase inhibitor. Non-limiting examples of HIF-1α-PH inhibitors can be found in U.S. Pat. No. 8,999,971, which is hereby incorporated by reference in its entirety. In certain embodiments, the HIF-1α-PH inhibitor is a compound having the formula:

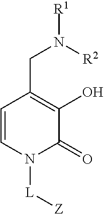

wherein L is chosen from CH₂ or SO₂;
Z has the formula:

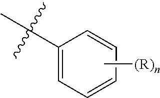

R represents from 0 to 5 substitutions for hydrogen;
the index n is an integer from 0 to 5;
R¹ and R² are each independently chosen from:
i) hydrogen;
ii) substituted or unsubstituted C₁-C₁₀ linear, C₃-C₁₀ branched, or C₃-C₁₀ cyclic alkyl;
iii) substituted or unsubstituted C₂-C₁₀ linear, C₃-C₁₀ branched, or C₃-C₁₀ cyclic alkenyl;
iv) substituted or unsubstituted C₂-C₁₀ linear or C₃-C₁₀ branched alkynyl;
v) substituted or unsubstituted C₆ or C₁₀ aryl;
vi) substituted or unsubstituted C₁-C₉ heterocyclic;
vii) substituted or unsubstituted C₁-C₉ heteroaryl; or
viii) R¹ and R² can be taken together to form a substituted or unsubstituted heterocyclic or substituted or unsubstituted heteroaryl ring having from form 2 to 20 carbon atoms and from 1 to 7 heteroatoms; or
a pharmaceutically acceptable salt thereof.

In various embodiments, L is CH₂.

In other various embodiments, L is SO₂.

In various embodiments, each R is a substitution for hydrogen independently chosen from:
i) substituted or unsubstituted C₁-C₁₂ linear, C₃-C₁₂ branched, or C₃-C₁₂ cyclic alkyl;
ii) substituted or unsubstituted C₁-C₁₂ linear, C₃-C₁₂ branched, or C₃-C₁₂ cyclic alkenyl;
iii) substituted or unsubstituted C₂-C₁₂ linear or C₃-C₁₂ branched alkynyl;
iv) C₆ or C₁₀ substituted or unsubstituted aryl;
v) C₁-C₉ substituted or unsubstituted heterocyclic;
vi) substituted or unsubstituted heteroaryl;
vii) halogen;
viii) —[C(R^{23a})(R^{23b})]ₓOR¹⁰;
R¹⁰ is chosen from:
   a) —H;
   b) substituted or unsubstituted C₁-C₁₂ linear, C₃-C₁₂ branched, or C₃-C₁₂ cyclic alkyl;
   c) C₆ or C₁₀ substituted or unsubstituted aryl or alkylenearyl;
   d) C₁-C₉ substituted or unsubstituted heterocyclic;
   e) substituted or unsubstituted heteroaryl;
ix) —[C(R^{23a})(R^{23b})]ₓN(R^{11a})(R^{11b});
R^{11a} and R^{11b} are each independently chosen from:
   a) —H;
   b) —OR¹²;
R¹² is hydrogen or C₁-C₄ linear alkyl;
   c) substituted or unsubstituted C₁-C₁₂ linear, C₃-C₁₂ branched, or C₃-C₁₂ cyclic alkyl;
   d) C₆ or C₁₀ substituted or unsubstituted aryl;
   e) C₁-C₉ substituted or unsubstituted heterocyclic;
   f) C₁-C₁₁ substituted or unsubstituted heteroaryl; or
   g) R^{11a} and R^{11b} can be taken together to form a substituted or unsubstituted ring having from 3 to 10 carbon atoms and from 0 to 3 heteroatoms chosen from oxygen, nitrogen, and sulfur;
x) —[C(R^{23a})(R^{23b})]^{x}C(O)R¹³;
R¹³ is:
   a) substituted or unsubstituted C₁-C₁₂ linear, C₃-C₁₂ branched, or C₃-C₁₂ cyclic alkyl;
   b) —OR¹⁴;
      R¹⁴ is hydrogen, substituted or unsubstituted C₁-C₄ linear alkyl, C₆ or C₁₀ substituted or unsubstituted aryl, C₁-C₉ substituted or unsubstituted heterocyclic, C₁-C₁₁ substituted or unsubstituted heteroaryl;
   c) —N(R^{15a})(R^{15b});
      R^{15a} and R^{15b} are each independently hydrogen, substituted or unsubstituted C₁-C₁₂ linear, C₃-C₁₂ branched, or C₃-C₁₂ cyclic alkyl; C₆ or C₁₀ substituted or unsubstituted aryl; C₁-C₆ substituted or unsubstituted heterocyclic; C₁-C₁₁ substituted or unsubstituted heteroaryl; or R^{15a} and R^{15b} can be taken together to form a substituted or unsubstituted ring having from 3 to 10 carbon atoms and from 0 to 3 heteroatoms chosen from oxygen, nitrogen, and sulfur;
xi) —[C(R^{23a})(R^{23b})]ₓOC(O)R¹⁶;
   R¹⁶ is:
      a) substituted or unsubstituted C₁-C₁₂ linear, C₃-C₁₂ branched, or C₃-C₁₂ cyclic alkyl;
      b) —N(R^{17a})(R^{17b});

R^{17a} and R^{17b} are each independently hydrogen, substituted or unsubstituted C₁-C₁₂ linear, C₃-C₁₂ branched, or C₃-C₁₂ cyclic alkyl; C₆ or C₁₀ substituted or unsubstituted aryl; C₁-C₆ substituted or unsubstituted heterocyclic; C₁-C₁₁ substituted or unsubstituted heteroaryl; or R^{17a} and R^{17b} can be taken together to form a substituted or unsubstituted ring having from 3 to 10 carbon atoms and from 0 to 3 heteroatoms chosen from oxygen, nitrogen, and sulfur;
xii) —[C(R^{23a})(R^{23b})]ₓNR¹⁸C(O)R¹⁹;
   R¹⁸ is:
      a) —H; or
      b) substituted or unsubstituted C₁-C₄ linear, C₃-C₄ branched, or C₃-C₄ cyclic alkyl;
   R¹⁹ is:
      a) substituted or unsubstituted C₁-C₁₂ linear, C₃-C₁₂ branched, or C₃-C₁₂ cyclic alkyl;
      b) N(R^{20a})(R^{20b});
         R^{20a} and R^{20b} are each independently hydrogen, substituted or unsubstituted C₁-C₁₂ linear, C₃-C₁₂ branched, or C₃-C₁₂ cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{20a} and R^{20b} can be taken together to form a substituted or unsubstituted ring having from 3 to 10 carbon atoms and from 0 to 3 heteroatoms chosen from oxygen, nitrogen, and sulfur;
xiii) —[C(R^{23a})(R^{23b})]ₓCN;
xiv) —[C(R^{23a})(R^{23b})]ₓNO₂;
xv) —[C(R^{23a})(R^{23b})]ₓR²¹;
   R²¹ is C₁-C₁₀ linear, branched, or cyclic alkyl substituted by from 1 to 21 halogen atoms chosen from —F, —Cl, —Br, or —I;
xvi) —[C(R^{23a})(R^{23b})]ₓSO₂R²²;
   R²² is hydrogen, hydroxyl, substituted or unsubstituted C₁-C₄ linear or C₃-C₄ branched alkyl; substituted or unsubstituted C₆, C₁₀, or C₁₋₄ aryl; C₇-C₁₅ alkylenearyl; C₁-C₉ substituted or unsubstituted heterocyclic; or C₁-C₁₁ substituted or unsubstituted heteroaryl;
   R^{23a} and R^{23b} are each independently hydrogen or C₁-C₄ alkyl; and
the index x is an integer from 0 to 5.
Z is 4-chlorophenyl.

In various embodiments, Z is chosen from 2-chlorophenyl, 3-chlorophenyl, 2-fluorophenyl, 3-fluorophenyl, or 4-fluorophenyl.

In certain embodiments, the HIF-1α prolyl hydroxylase inhibitor is chosen from:
1-Benzyl-3-hydroxy-4-(piperidin-1-ylmethyl)pyridin-2(1H)-one;
1-Benzyl-3-hydroxy-4-(morpholin-4-ylmethyl)pyridin-2(1H)-one;
1-Benzyl-3-hydroxy-4-(thiomorpholin-4-ylmethyl)pyridin-2(1H)-one;
1-Benzyl-3-hydroxy-4-(thiazolidin-3-ylmethyl)pyridin-2(1H)-one;
1-Benzyl-3-hydroxy-4-(pyrrolidin-1-ylmethyl)pyridin-2(1H)-one;
1-Benzyl-3-hydroxy-4-(4-benzylpiperidin-1-ylmethyl)pyridin-2(1H)-one;
1-Benzyl-3-hydroxy-4-(4-benzylpiperazin-1-ylmethyl)pyridin-2(1H)-one;
1-Benzyl-3-hydroxy-4-[(3-hydroxypyrrolidin-1-yl)methyl]pyridin-2(1H)-one;
1-Benzyl-3-hydroxy-4-(1,4-dioxa-8-azaspiro[4,5]dec-8-ylmethyl)pyridin-2(1H)-one;
1-Benzyl-3-hydroxy-4-azepan-1-ylmethylpyridin-2(1H)-one;
1-Benzyl-3-hydroxy-4-(azocan-1-ylmethyl)pyridin-2(1H)-one;
1-Benzyl-3-hydroxy-(1,4′-bipiperidinyl-1′-ylmethyl)pyridin-2(1H)-one;
1-Benzyl-3-hydroxy-4-[(3,4-dihydroquinolin-1(2H)-yl)methyl]pyridin-2(1H)-one;
Methyl 1-[(1-benzyl-3-hydroxy-2-oxo-1,2-dihydropyridin-4-yl)methyl]pyrrolidine-2-carboxylate;
1-Benzyl-3-hydroxy-4-{[2-(methoxymethyl)pyrrolidin-1-yl]methyl}pyridin-2(1H)-one;
1-Benzyl-3-hydroxy-4-{[2-(pyrdin-2-yl)pyrrolidin-1-yl]methyl}pyridin-2(1H)-one;
1-Benzyl-3-hydroxy-4-[4-(6-chloropyridazin-3-yl)piperazin-1-ylmethyl]pyridin-2(1H)-one;
1-Benzyl-3-hydroxy-4-[4-(2-methoxyphenyl)piperazin-1-ylmethyl]pyridin-2(1H)-one;
1-(3-Methoxybenzyl)-3-hydroxy-4-(piperidin-1-ylmethyl)pyridin-2(1H)-one;
1-Benzyl-3-hydroxy-4-{[3-(1-H-imidazol-1-yl)propylamino]methyl}pyridin-2(1H)-one;
1-Benzyl-3-hydroxy-4-(benzylaminomethyl)pyridin-2(1H)-one;
1-Benzyl-3-hydroxy-4-{[(2-(pyridin-2-yl)ethylamino]methyl}pyridin-2(1H)-one;
1-Benzyl-3-hydroxy-4-{[(tetrahydrofuran-2-ylmethyl)amino]methyl}pyridin-2(1H)-one;
1-Benzyl-3-hydroxy-4-[(2-methoxyethylamino)methyl]pyridin-2(1H)-one;
1-Benzyl-3-hydroxy-4-[(1-hydroxy-2-methylpropan-2-ylamino)methyl]pyridin-2(1H)-one;
1-Benzyl-3-hydroxy-4-[(pyridin-4-ylmethylamino)methyl]pyridin-2(1H)-one;
1-Benzyl-3-hydroxy 4-{[(furan-2-ylmethyl)amino]methyl}pyridin-2(1H)-one;
1-Benzyl-3-hydroxy-4-{[2-(methylthio)ethylamino]methyl}pyridin-2(1H)-one;
1-Benzyl-3-hydroxy-4-[(4-methoxybenzylamino)methyl]pyridin-2(1H)-one;
1-Benzyl-3-hydroxy-4-[(1-phenylethylamino)methyl]pyridin-2(1H)-one;
1-Benzyl-3-hydroxy-4-(cycloheptylaminomethyl)pyridin-2(1H)-one;
1-Benzyl-3-hydroxy-4-[(4-methylcyclohexylamino)methyl]pyridin-2(1H)-one;
1-Benzyl-3-hydroxy-4-[(1-benzylpiperidin-4-ylamino)methyl]pyridin-2(1H)-one;
3-[(1-Benzyl-3-hydroxy-2-oxo-1,2-dihydropyridin-4-yl)methylamino]azepan-2-one;
1-Benzyl-3-hydroxy-4-[(1-benzylpyrrolidin-3-ylamino)methyl]pyridin-2(1H)-one;
(R)-1-Benzyl-3-hydroxy-4-[(1-phenylethylamino)methyl]pyridin-2(1H)-one;
1-Benzyl-3-hydroxy-4-[([1,3]dioxolan-2-ylmethylmethylamino)methyl]pyridin-2(1H)-one;
1-(4′-Methylbenzenesulfonyl)-3-hydroxy-4-(pyrrolidin-1-ylmethyl)pyridin-2(1H)-one;
1-(4′-Methylbenzenesulfonyl)-3-hydroxy-4-thiazolidin-3-ylmethylpyridin-2(1H)-one;
1-(4′-Methylbenzenesulfonyl)-3-hydroxy-4-azocan-1ylmethylpyridin-2(1H)-one;
1-(4′-Methylbenzenesulfonyl)-3-hydroxy-4-(4-phenylpiperazin-1-ylmethyl)-pyridin-2(1H)-one;
1-(4′-Methylbenzenesulfonyl)-3-hydroxy-4-[1,4′]bipiperidinyl-1′-ylmethylpyridin-2(1H)-one;
1-(4′-Methylbenzenesulfonyl)-3-hydroxy-4-[4-(6-chloropyridazin-3-yl)piperazin-1-ylmethyl]pyridin-2(1H)-one;
1-(4′-Methylbenzenesulfonyl)-3-hydroxy-4-(benzylaminomethyl)pyridin-2(1H)-one; or
1-(4′-Methylbenzenesulfonyl)-3-hydroxy-4-[(2-methoxyethylamino)methyl]-pyridin-2(1H)-one.

In certain embodiments, the HIF-1α prolyl hydroxylase inhibitor has the formula:

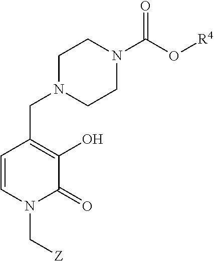

wherein Z is phenyl substituted with from 1 to 5 halogens chosen from fluorine and chlorine;
R⁴ is C₁-C₄ linear alkyl or C₃-C₄ branched alkyl;
or a pharmaceutically acceptable salt thereof.

In various embodiments, R4 is tert-butyl.

In various embodiments, Z is 4-chlorophenyl.

In certain embodiments, the one or more compounds is tert-butyl 4-{[1-(4-chlorobenzyl)-3-hydroxy-2-oxo-1,2-dihydropyridin-4-yl]methyl}-piperazine-1-carboxylate or a pharmaceutically acceptable salt chosen from hydrochloride salt, hydrogen sulfate salt, sulfate salt, p-toluenesulfonyl salt, methansulfonyl salt and mixtures thereof.

In some embodiments, the HIF-PH inhibitor is an iron chelator, 2-oxoglutarate mimetics, and modified amino acid, e.g., proline analogs, or compounds that function as 2-oxoglutarate mimetics. In certain embodiments, the 2-oxoglutarate mimetic is a heterocyclic carboxamide. In certain embodiments, the heterocyclic carboxamide is a structural mimetic of 2-oxoglutarate. In certain embodiments, the heterocyclic carboxamide compound is a heterocyclic carbonyl glycine compound. In various embodiments, the heterocyclic carboxamide is a quinoline carboxamide, an isoquinoline carboxamide, a pyridine carboxamide, a cinnoline carboxamide, or a beta-carboline carboxamide. Non-limiting examples of HIF-PH inhibitors can be found in U.S. Pat. No. 9,775,902, which is hereby incorporated by reference in its entirety. In certain embodiments, the HIF-PH inhibitor can include, but is not limited to: [(1-Chloro-4-hydroxy-isoquinoline-3-carbonyl)-amino]-acetic acid; [(7-Chloro-3-hydroxy-quinoline-2-carbonyl)-amino]-acetic acid; [(3-Hydroxy-6-phenoxy-quinoline-2-carbonyl)-amino]-acetic acid; [(1-Chloro-4-hydroxy-5-methyl-isoquinoline-3-carbonyl)-amino]-acetic acid; [(4-Hydroxy-7-phenylsulfanyl-isoquinoline-3-carbonyl)-amino]-acetic acid; {[4-Hydroxy-7-(4-methoxy-phenoxy)-isoquinoline-3-carbonyl]-amino}-acetic acid; {[7-(4-Fluoro-phenoxy)-4-hydroxy-isoquinoline-3-carbonyl]-amino}-acetic acid; {[1-Chloro-4-hydroxy-6-(4-methoxy-phenoxy)-isoquinoline-3-carbonyl]-amino}-acetic acid; 2-[(4-Hydroxy-7-phenoxy-isoquinoline-3-carbonyl)-amino]-propionic acid; [(4-Hydroxy-1-methyl-7-phenoxy-isoquinoline-3-carbonyl)-amino]-acetic acid; [(4-Benzyloxy-1-methyl-7-phenoxy-isoquinoline-3-carbonyl)-amino]-acetic acid; [(4-Chloro-7-hydroxy-thieno[3,2-c]pyridine-6-carbonyl)-amino]-acetic acid; [(7-Ethynyl-4-hydroxy-thieno[2,3-c]pyridine-5-carbonyl)-amino]-acetic acid; {[4-Hydroxy-7-(2-methyl-benzooxazol-6-yloxy)-isoquinoline-3-carbonyl]-amino}-acetic acid; {[7-(Benzo[1,3]dioxol-5-yloxy)-4-hydroxy-isoquinoline-3-carbonyl]-amino}-acetic acid; {[2-(4-Fluoro-phenyl)-4-hydroxy-7-methyl-thieno[2,3-c]pyridine-5-carbonyl]-amino}-acetic acid; {[2-(4-Chloro-phenyl)-6-hydroxy-thieno[3,2-b]pyridine-5-carbonyl]-amino}-acetic acid; {[1-Cyano-7-(4-fluoro-phenoxy)-4-hydroxy-isoquinoline-3-carbonyl]-amino}-acetic acid; [(7-Chloro-1-cyano-4-hydroxy-isoquinoline-3-carbonyl)-amino]-acetic acid; [(7-Chloro-3-hydroxy-4-iodo-quinoline-2-carbonyl)-amino]acetic acid; {[1-(4-Chloro-phenylsulfanyl)-4-hydroxy-isoquinoline-3-carbonyl]-amino}-acetic acid; [(7-Cyclohexylsulfanyl-4-hydroxy-isoquinoline-3-carbonyl)-amino]-acetic acid; [(1-Cyano-4-hydroxy-8-phenoxy-isoquinoline-3-carbonyl)-amino]-acetic acid; {[7-(2,3-Dihydro-benzofuran-5-yloxy)-4-hydroxy-isoquinoline-3-carbonyl]-amino}-acetic acid; 2-[(4-Hydroxy-7-phenylsulfanyl-isoquinoline-3-carbonyl)-amino]-propionic acid; {[1-(2-Fluoro-phenoxy)-4-hydroxy-isoquinoline-3-carbonyl]-amino}-acetic acid; [(4-Hydroxy-1-methyl-6-phenoxy-isoquinoline-3-carbonyl)-amino]-acetic acid; {[4-Hydroxy-6-(pyridin-2-ylsulfanyl)-isoquinoline-3-carbonyl]-amino}-acetic acid; [(4-Hydroxy-7-phenoxy-isoquinoline-3-carbonyl)-amino]acetic acid; [(2,4-Dibromo-7-hydroxy-thieno[3,2-c]pyridine-6-carbonyl)-amino]-acetic acid; [(4-Bromo-7-hydroxy-thieno[3,2-c]pyridine-6-carbonyl)-amino]-acetic acid; {[4-Hydroxy-1-methyl-7-(2-methyl-benzooxazol-6-yloxy)-isoquinoline-3-carbonyl]-amino }-acetic acid; [(7-Hydroxy-2-phenoxy-thieno[3,2-c]pyridine-6-carbonyl)-amino]-acetic acid; [(4-Cyano-7-hydroxy-thieno[3,2-c]pyridine-6-carbonyl)-amino]-acetic acid; [(4-Furan-3-yl-7-hydroxy-thieno[3,2-c]pyridine-6-carbonyl)-amino]-acetic acid; {[2,3-Bis-(4-fluoro-phenyl)-7-hydroxy-thieno[3,2-c]pyridine-6-carbonyl]-amino}-acetic acid; [(1-Formyl-4-hydroxy-6-phenoxy-isoquinoline-3-carbonyl)-amino]-acetic acid; {[1-Cyano-6-(2,6-dimethyl-phenoxy)-4-hydroxy-isoquinoline-3-carbonyl]-amino}-acetic acid; [(1-Cyano-4-hydroxy-5-phenoxy-isoquinoline-3-carbonyl)-amino]-acetic acid; {[6-(Benzo[1,3]dioxol-5-yloxy)-1-cyano-4-hydroxy-isoquinoline-3-carbonyl]-amino}-acetic acid; {[1-Cyano-6-(2,3-dihydro-benzofuran-5-yloxy)-4-hydroxy-isoquinoline-3-carbonyl]-amino}-acetic acid; {[1-Cyano-4-hydroxy-8-(3-methoxy-phenoxy)-isoquinoline-3-carbonyl]-amino}-acetic acid; {[1-Cyano-4-hydroxy-6-(2-methyl-benzooxazol-6-yloxy)-isoquinoline-3-carbonyl]-amino}-acetic acid; [(7-Benzyl-1-cyano-4-hydroxy-isoquinoline-3-carbonyl)-amino]-acetic acid; {[1-Cyano-5-(4-fluoro-phenoxy)-4-hydroxy-isoquinoline-3-carbonyl]-amino}-acetic acid; [(7-Chloro-4-ethyl-3-hydroxy-quinoline-2-carbonyl)-amino]-acetic acid; {[7-Chloro-3-hydroxy-4-(3-trifluoromethyl-phenyl)-quinoline-2-carbonyl]-amino}-acetic acid; [(6,7-Dichloro-4-hydroxy-isoquinoline-3-carbonyl)-amino]-acetic acid; [(4-Hydroxy-8-phenyl-isoquinoline-3-carbonyl)-amino]-acetic acid; [(4-Hydroxy-6,7-diphenoxy-isoquinoline-3-carbonyl)-amino]-acetic acid; {[7-(4-Fluoro-phenoxy)-4-hydroxy-1-methyl-isoquinoline-3-carbonyl]-amino}-acetic acid; [(1-Cyano-4-hydroxy-7-phenoxy-isoquinoline-3-carbonyl)-amino]-acetic acid; {[8-(4-Fluoro-phenoxy)-4-hydroxy-1-methyl-isoquinoline-3-carbonyl]-amino}-acetic acid; {[1-Cyano-8-(4-fluoro-phenoxy)-4-hydroxy-isoquinoline-3-carbonyl]-amino}-acetic acid; [(1-Cyano-4-hydroxy-6-phenoxy-isoquinoline-3-carbonyl)-amino]-acetic acid; and {[1-Cyano-6-(4-fluoro-phenoxy)-4-hydroxy-isoquinoline-3-carbonyl]-amino}-acetic acid.

In some embodiments, the HIF-PH inhibitor is a compound selected from: [(1-Chloro-4-hydroxy-isoquinoline-3-carbonyl)-amino]-acetic acid, [(4-Hydroxy-7-phenoxy-isoquinoline-3-carbonyl)-amino]-acetic acid, [(4-Hydroxy-7-phenylsulfanyl-isoquinoline-3-carbonyl)-amino]-acetic acid, and 3-{([4-(3,3-Dibenzyl-ureido)-benzenesulfonyl]-[2-(4-methoxy-phenyl)-ethyl]-amino}-N-hydroxy-propionamide. Non-limiting examples of HIF-PH inhibitors that can be used in the present disclosure can be found in U.S. Pat. No. 8,629,131, which is hereby incorporated by reference in its entirety.

In some embodiments, the HIF-PH inhibitor is 1-(6-(2,6-dimethylphenoxy)-7-fluoro-4-oxo-3,4-dihydroquinazolin-2-yl)-1H-pyrazole-4-carboxylic acid (JNJ-42905343). In certain embodiments, JNJ-42905343 comprises a compound of formula:

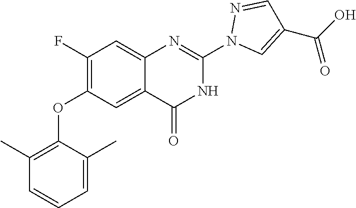 [SMILES: Cc1cccc(C)c1Oc1cc2c(=O)[nH]c(-n3cc(C(=O)O)cn3)nc2cc1F]

In some embodiments, the HIF-PH inhibitor is 1-(5-Chloro-6-(trifluoromethoxy)-1H-benzoimidazol-2-yl)-1H-pyrazole-4-carboxylic Acid (JNJ-42041935).

### 5.3.5. Pharmaceutical Composition

The HIF-PH inhibitor used in the methods described herein can be formulated in any appropriate pharmaceutical composition for administration by any suitable route of administration. Suitable routes of administration include, but are not limited to, oral and intravenous routes of administration. Suitable routes also include pulmonary administration, including by oral inhalation. The most suitable route may depend upon the condition and disorder of the recipient. The formulations may conveniently be presented in unit dosage form and may be prepared by any of the methods known in the art of pharmacy.

All methods include the step of bringing into association a HIF-PH inhibitor, or a salt thereof, with the carrier which constitutes one or more excipients. In general, the formulations are prepared by uniformly and intimately bringing into association the active ingredient with liquid carriers or finely divided solid carriers or both and then, if necessary, shaping the product into the desired formulation.

In certain embodiments, the route of administration for use in the methods described herein is parental administration. In certain embodiments, the route of administration for use in the methods described herein is intravenous administration. In certain embodiments, the route of administration for use in the methods described herein is oral administration.

Formulations of the present methods suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein.

Formulations for parenteral administration include aqueous and non-aqueous sterile injection solutions which may contain antioxidants, buffers, bacteriostats and solutes which render the formulation isotonic with the blood of the intended recipient. Formulations for parenteral administration also include aqueous and non-aqueous sterile suspensions, which may include suspending agents and thickening agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described.

The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, 8th Revised Ed. (2017), incorporated by reference in its entirety.

The term “pharmaceutically acceptable salt” refers to salts prepared from pharmaceutically acceptable non-toxic acids or bases including inorganic acids and bases and organic acids and bases. Non-limiting examples of pharmaceutically acceptable salts include, but are not limited to acid addition salts including mineral acid salts such as hydrochloride, hydrobromide, hydroiodide, phosphate, sulfate, and nitrate; sulfonic acid salts such as methanesulfonate, ethanesulfonate, benzenesulfonate, p-toluenesulfonate, and trifluoromethanesulfonate; organic acid salts such as oxalate, tartrate, citrate, maleate, succinate, acetate, trifluoroacetate, benzoate, mandelate, ascorbate, lactate, gluconate, and malate; amino acid salts such as glylcine salt, lysine salt, arginine salt, ornithine salt, glutamate, and aspartate; inorganic salts such as lithium salt, sodium salt, potassium salt, calcium salt, and magnesium salt; and salts with organic bases such as ammonium salt, triethylamine salt, diisopropylamine salt, and cyclohexylamine salt.

### 5.3.6. Dosage Regimens

In various embodiments, the HIF-PH inhibitor is administered at a dose sufficient to treat an age-related condition, such as, but not limited to, frailty, anemia, anemia with chronic kidney disease, anemia of aging, fatigue, fibrosis, inflammation, muscle aging, hip fracture/hip fracture functional recovery, post-ICU functional recovery sarcopenia, tissue injury, and ischemic damage.

In some embodiments, the HIF-PH inhibitor is administered in an amount of at least 0.001 mg/kg. In certain embodiments, the HIF-PH inhibitor is administered in an amount of at least 0.01 mg/kg. In certain embodiments, the HIF-PH inhibitor is administered in an amount of at least 0.05 mg/kg. In certain embodiments, the HIF-PH inhibitor is administered in an amount of at least 0.5 mg/kg. In certain embodiments, the HIF-PH inhibitor is administered orally in an amount of at least 1 mg/kg. In certain embodiments, the dose is at least 2 mg/kg, at least 3 mg/kg, at least 4 mg/kg, at least 5 mg/kg, at least 6 mg/kg, at least 7 mg/kg, at least 8 mg/kg, at least 9 mg/kg, or at least 10 mg/kg.

In various embodiments, the dose of the HIF-PH inhibitor is at least 0.01 mg/kg. In various embodiments, the dose of the HIF-PH inhibitor is at least 0.1 mg/kg. In various embodiments, the dose of the HIF-PH inhibitor is at least 0.5 mg/kg. In various embodiments, the dose of the HIF-PH inhibitor is at least 1 mg/kg. In various embodiments, the dose of the HIF-PH inhibitor is at least 1.5 mg/kg, at least 2 mg/kg, at least 2.5 mg/kg, at least 3 mg/kg, at least 3.5 mg/kg, at least 4 mg/kg, at least 4.5 mg/kg, at least 5 mg/kg, at least 5.5 mg/kg, at least 6 mg/kg, at least 6.5 mg/kg, at least 7 mg/kg, at least 7.5 mg/kg, at least 8 mg/kg, at least 8.5 mg/kg, at least 9 mg/kg, at least 9.5 mg/kg, or at least 10 mg/kg. In certain embodiments, the dose is at least 5 mg/kg, at least 10 mg/kg at least 15 mg/kg, at least 20 mg/kg, at least 25 mg/kg, 30 mg/kg, at least 35 mg/kg, at least 40 mg/kg, at least 45 mg/kg, at least 50 mg/kg, at least 55 mg/kg, at least 60 mg/kg, at least 65 mg/kg, at least 70 mg/kg, at least 75 mg/kg, at least 80 mg/kg, at least 85 mg/kg, at least 90 mg/kg, at least 95 mg/kg, at least 100 mg/kg, at least 125 mg/kg, at least 150 mg/kg, at least 160 mg/kg, at least 175 mg/kg, or at least 200 mg/kg. In certain embodiments, the dose is 250 mg/kg, 300 mg/kg, 350 mg/kg, 400 mg/kg, 450 mg/kg, 500 mg/kg, 600 mg/kg, 650 mg/kg, 700 mg/kg, 750 mg/kg, 800 mg/kg, 850 mg/kg, 900 mg/kg, 950 mg/kg, or 1000 mg/kg. In certain embodiments, the dose is 0.001 mg/kg to 100 mg/kg per day. In certain embodiments, the dose is 2 mg/kg to 100 mg/kg per day. In certain embodiments, the dose is 25 mg/kg to 1000 mg/kg per day.

In certain embodiments, the dose is at least 0.01 mg/kg PO per day. In certain embodiments, the dose is at least 0.1 mg/kg PO per day. In certain embodiments, the dose is at least 0.05 mg/kg PO per day. In certain embodiments, the dose is at least 0.5 mg/kg PO per day. In certain embodiments, the dose is at least 1 mg/kg PO per day. In certain embodiments, the dose is at least 2 mg/kg PO per day. In certain embodiments, the dose is at least 3 mg/kg PO per day. In certain embodiments, the dose is at least 4 mg/kg PO per day. In certain embodiments, the dose is at least 5 mg/kg PO per day. In certain embodiments, the dose is at least 6 mg/kg PO per day. In certain embodiments, the dose is at least 7 mg/kg PO per day. In certain embodiments, the dose is at least 8 mg/kg PO per day. In certain embodiments, the dose is at least 9 mg/kg PO per day. In certain embodiments, the dose is at least 10 mg/kg PO per day. In certain embodiments, the dose is at least 11 mg/kg PO per day. In certain embodiments, the dose is at least 12 mg/kg PO per day. In certain embodiments, the dose is at least 13 mg/kg PO per day.

In various embodiments, the dose of the HIF-PH inhibitor is at least 0.5 mg/kg. In certain embodiments, the dose is at least 1 mg/kg. In certain embodiments, the dose is at least 40 mg/kg, at least 40 mg/kg, at least 50 mg/kg, at least 100 mg/kg, at least 150 mg/kg, at least 175 mg/kg, or at least 200 mg/kg. In certain embodiments, the dose is 250 mg/kg, 500 mg/kg, 750 mg/kg, or 1000 mg/kg. In certain embodiments, the dose is 25 mg/kg to 1,000 mg/kg per day.

In some embodiments, the HIF-PH inhibitor is administered at a dose of 0.001 mg/kg, 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg or 0.1 mg/kg. In some embodiments, the HIF-PH inhibitor is administered at a dose of 0.1 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, or 1.0 mg/kg. In some embodiments, the HIF-PH inhibitor is administered at a dose of 1.5 mg/kg, 2 mg/kg, 2.5 mg/kg, 3 mg/kg, 3.5 mg/kg, 4 mg/kg, 4.5 mg/kg, or 5 mg/kg. In some embodiments, the HIF-PH inhibitor is administered at a dose of 6 mg/kg, 7 mg/kg, 8 mg/kg, 9 mg/kg, 10 mg/kg, 12 mg/kg, 15 mg/kg, 20 mg/kg, 30 mg/kg, 40 mg/kg, or 50 mg/kg. In some embodiments, the HIF-PH inhibitor is administered at a dose of 10 mg/kg, 50 mg/kg, 8 mg/kg, 100 mg/kg, 150 mg/kg, 200 mg/kg, 250 mg/kg, 300 mg/kg, 350 mg/kg, 400 mg/kg, or 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, or 1000 mg/kg.

In some embodiments, the HIF-PH inhibitor is administered in a dose that is independent of patient weight or surface area (flat dose).

In some embodiments, the flat dose is 0.001 mg, 0.01 mg, 0.1 mg, 0.2 mg, 0.3 mg, 0.4 mg, 0.5 mg, 0.6 mg, 0.7 mg, 0.8 mg, 0.9 mg, or 1 mg. In some embodiments, the flat dose is 1 mg, 2 mg, 3 mg, 4 mg, 5 mg, 6 mg, 7 mg, 8 mg, 9 mg, or 10 mg. In some embodiments, the flat dose is 11 mg, 12 mg, 13 mg, 14 mg, 15 mg, 16 mg, 17 mg, 18 mg, 19 mg, or 20 mg. In some embodiments, the flat dose is 25 mg, 30 mg, 35 mg, 40 mg, 45 mg, 46 mg, 47 mg, 48 mg, 49 mg, or 50 mg. In some embodiments, the flat dose is 40 mg, 42 mg, 44 mg, 46 mg, 48 mg, 50 mg, 60 mg, 70 mg, 80 mg, 90 mg, or 100 mg. In some embodiments, the flat dose is 200 mg, 300 mg, 400 mg, 500 mg, 600 mg, 700 mg, 800 mg, 900 mg, or 1000 mg. In some embodiments, the flat dose ranges from 0.1 to 40 mg. In some embodiments, the flat dose ranges from 12 to 30 mg. In some embodiments, the flat dose is 0.1-1 mg, 1-10 mg, 10-15 mg, 15-20 mg, 20-30 mg, 30-40 mg, or 40-50 mg. In some embodiments, the flat dose is 1-50 mg, 50-100 mg, 100 mg-200 mg, 200 mg-300 mg, 300 mg-400 mg, 400 mg-500 mg, 500 mg-600 mg, 600 mg-700 mg, 700 mg-800 mg, 800 mg-900 mg, or 900 mg-1000 mg.

In various embodiments, the dose is 1-5000 mg. In various embodiments, the flat dose is 12-30 mg. In various embodiments, the flat dose is 1-11 mg. In various embodiments, the flat dose is 12-40 mg. In certain embodiments, the dose is 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 450 mg, 500 mg, 550 mg, 600 mg, 650 mg, 700 mg, 750 mg, 800 mg, 850 mg, 900 mg, 950 mg, or 1000 mg. In certain embodiments, the dose is 1500 mg, 2000 mg, 2500 mg, 3000 mg, 3500 mg, 4000 mg, 4500 mg, or 5000 mg.

In various embodiments, the dose is 25-2000 mg. In certain embodiments, the dose is 25 mg, 50 mg, 75 mg, 100 mg, 125 mg, 150 mg, 175 mg, 200 mg, 225 mg, 250 mg, 275 mg, 300 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 900 mg, 925 mg, 950 mg, 975 mg, or 1000 mg.

The HIF-PH inhibitor can be administered in a single dose or in multiple doses. In various embodiments, the HIF-PH inhibitor is administered once a day, once every 2 days, once every 3 days, once every 4 days, once every 5 days, once every 6 days, once every 7 days, once every 14 days, once every 21 days, once every 28 days, or once a month. In various embodiments, HIF-PH inhibitor is administered twice a day, twice every 2 days, twice every 3 days, twice every 4 days, twice every 5 days, twice every 6 days, twice every 7 days, twice every 14 days, twice every 21 days, twice every 28 days, or twice a month. In various embodiments, the HIF-PH inhibitor is administered 1 time a week, 2 times a week, 3 times a week, four times a week, or five times a week.

### 5.3.7. Dosage Form

In some embodiments, a HIF-PH inhibitor or salt thereof is administered in a suspension. In other embodiments, a HIF-PH inhibitor or salt thereof is administered in a solution. In some embodiments, a HIF-PH inhibitor or salt thereof is administered in a solid dosage form. In particular embodiments, the solid dosage form is a capsule. In particular embodiments, the solid dosage form is a tablet. In specific embodiments, a HIF-PH inhibitor is in a crystalline or amorphous form. In particular embodiments, a HIF-PH inhibitor is in amorphous form.

### 5.4. EXAMPLES

Below are examples of specific embodiments for carrying out the present disclosure. The examples are offered for illustrative purposes only, and are not intended to limit the scope of the present disclosure in any way. Efforts have been made to ensure accuracy with respect to numbers used (e.g., amounts, temperatures, etc.), but some experimental error and deviation should, of course, be allowed for.

The practice of the present disclosure will employ, unless otherwise indicated, conventional methods of protein chemistry, biochemistry, recombinant DNA techniques and pharmacology, within the skill of the art. Such techniques are explained fully in the literature.

### 5.4.1. Example 1

### Bioinformatic Analyses Identify Relationships of HIF1α and HIF-PH to All-Cause Mortality (Survival) and to Mobility Decline Events in Human Healthy Aging Cohorts

A survival predictor model was used to examine the relationship between serum levels of HIF1α and HIF-PH and future risk of all-cause mortality in human healthy aging cohorts, using clinical outcome data from those cohorts and proteomics data generated on archived samples, based on survival modeling. Additionally, the relationship between HIF1α and HIF-PH levels and mobility decline events (e.g., a decrease in ability of walking, stair-climbing, or transferring activities indicated by self-reported difficulty of these activities) were examined using a Cox proportional hazards model, with a hazard ratio and associated p-value generated for each of HIF1α and HIF PH.

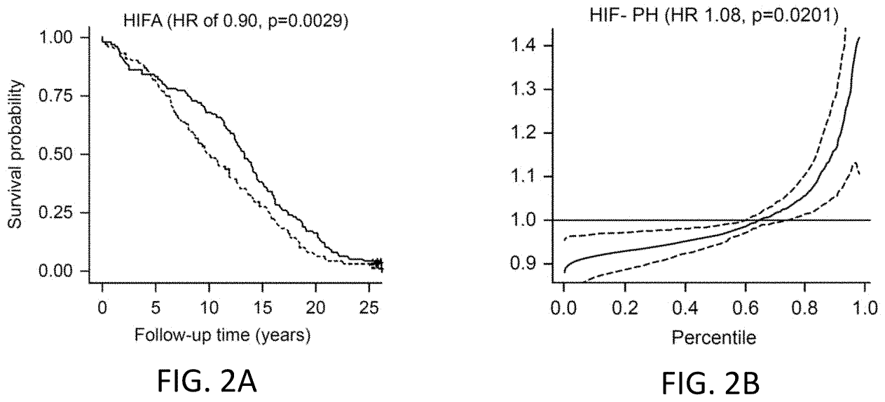

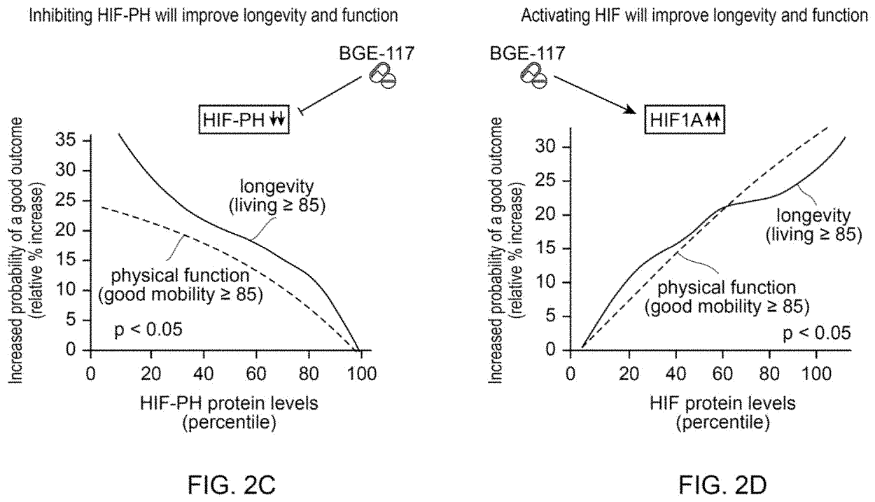

As shown in FIG. 2A, a Kaplan-Meier curve of survival probability was generated for humans in the top 20% (solid line) versus bottom 20% (dashed line) of HIF-1α protein levels. In humans, we have discovered that higher circulating levels of HIF1α are associated with decreased all-cause mortality (p=0.0029). FIG. 2B shows a similar model, used with restricted cubic splines to generate a non-linear fit of survival hazard ratio by protein levels, where we discovered that higher circulating levels of HIF-PH are associated with increased all-cause mortality (p=0.0201). The dashed lines show the 95% confidence interval for the solid line. The hazard ratio for HIF1α (0.90) and HIF PH (1.08) was generated using a Cox proportional hazards model. p-values in FIGS. 2A and 2B were calculated for these hazard ratios, based on testing the null hypothesis that the hazard ratio in each case equals 1.

The hazard ratio of HIF1α and HIF-PH of the survival probability model showed that higher levels of HIF1α are associated with better future physical function, and higher levels of HIF-PH are associated with worse future physical function.

Similarly, FIGS. 2C-2D demonstrate the HIF pathway involvement in the etiology of aging-related morbidity. FIG. 2D shows that higher levels of HIF1α (x axis) are correlated with improved longevity (living ≥85 years) and physical function (good mobility ≥85 years) as depicted by increased probability of good outcome (y axis). FIG. 2C shows that lower levels of HIF-PH (x axis) are correlated with improved longevity (living ≥85 years) and physical function (good mobility ≥85 years) as depicted by increased probability of good outcome (y axis).

### 5.4.2. Example 2

### Downstream HIF1α Target Genes are Affected by HIF-1α Levels in Healthy Aged Humans

Based on the discovery of the association of baseline HIF1α pathway protein levels with future aging outcomes in otherwise healthy, aged, humans as described in Example 1, the same cohort was used to determine the effect of age on protein serum levels of HIF-1α, and on protein serum levels of known HIF-1α target genes.

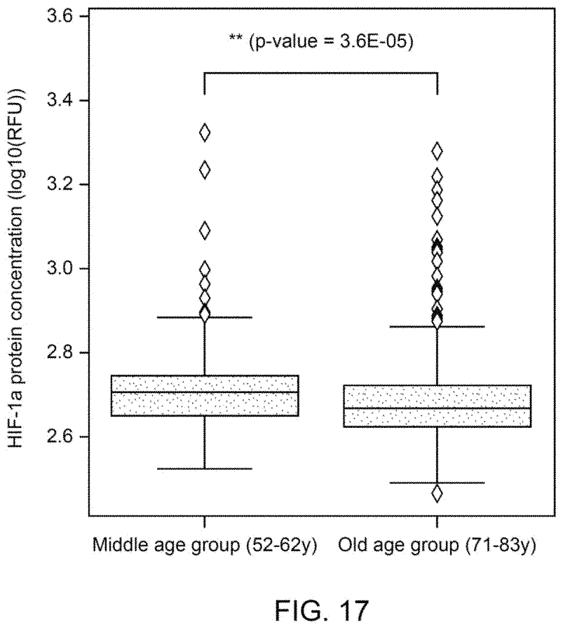

As shown in FIG. 17, HIF-1α serum protein concentration decreases with age in the human healthy aging cohorts of FIG. 2A (Middle aged group: 52-62 years old; and Old aged group: 71-83 years old).

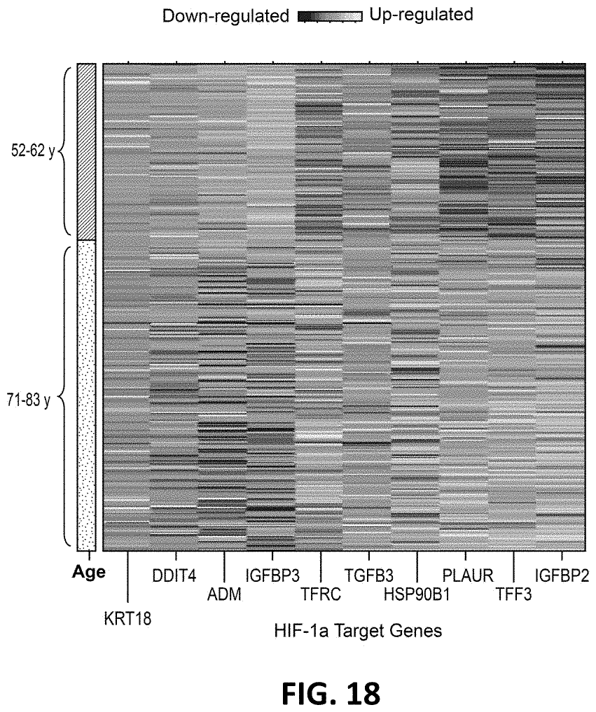

The heat map in FIG. 18 shows upregulation or downregulation of serum protein levels encoded by selected downstream target genes of HIF-1α (KRT18, DDT4, ADM, IGFBP3, TFRC, TGFB3, HSP0B1, PLAUR, TFF3, or IGFBP2) in the same individual during two timepoints: a timepoint between the ages of 52-62 years and a second timepoint between the ages of 71-83 years (timepoint is approximately 20 years apart). As can be seen in the heat map in FIG. 18, the difference of serum protein expression in the heat map shows that the levels (e.g., upregulation or downregulation) of downstream HIF-1α genes are affected when the level of HIF-1α changes with age. Thus, the HIF-1α signaling pathway is less activated in elderly humans due to a decrease in HIF-1α.

### 5.4.3. Example 3

### BGE-117 Improves Activity Levels in Old (Aged) Mice

Based on the discovery of (i) the association of baseline HIF1α pathway protein levels with future aging outcomes in otherwise healthy, aged, humans as described in Example 1 and (ii) decreases during aging in levels of HIF1α and its downstream targets as described in Example 2, an inhibitor of HIF prolyl hydroxylase was administered to elderly mice to assess the effects of the inhibitor on voluntary physical activity as compared to age-matched controls.

BGE-117 (also known as TP0463518 and TP518) has the structure shown below:

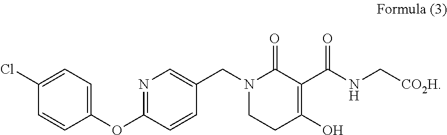 [SMILES: O=C(O)CNC(=O)C1=C(O)CCN(Cc2ccc(Oc3ccc(Cl)cc3)nc2)C1=O]

Although BGE-117 is known to inhibit HIF-PH and increase EPO production in patients with chronic kidney disease and in normal healthy human volunteers (Shinfuku et al., Am. J. Nephrol. 48(3):157-164 (2018)), and has been shown to increase hemoglobin levels in 5/6 nephrectomized rats (Kato et al., J. Pharmacol. Exp. Ther. 371:675-683 (2019), its effects on aged individuals with normal kidney function is unknown.

In this study, aged mice were treated with BGE-117 daily for 35 days. Daily voluntary running wheel activity levels, as well as hemoglobin levels at Days 0 and 14, were measured. The effects of the BGE-117 compound were examined for the reversal of frailty in mice.

### Study Design

In this experiment, 27-month-old mice (27 months old at the start of the experiment and 28 months old at the end since the experiment lasted 35 days), which were quite elderly and showed decreased Hb levels and activity levels relative to baseline, were used.

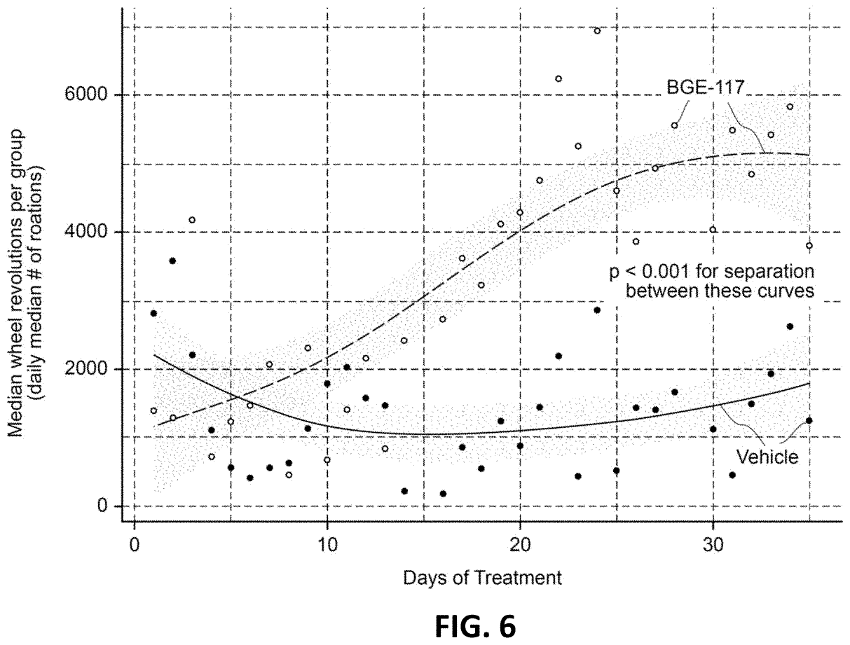

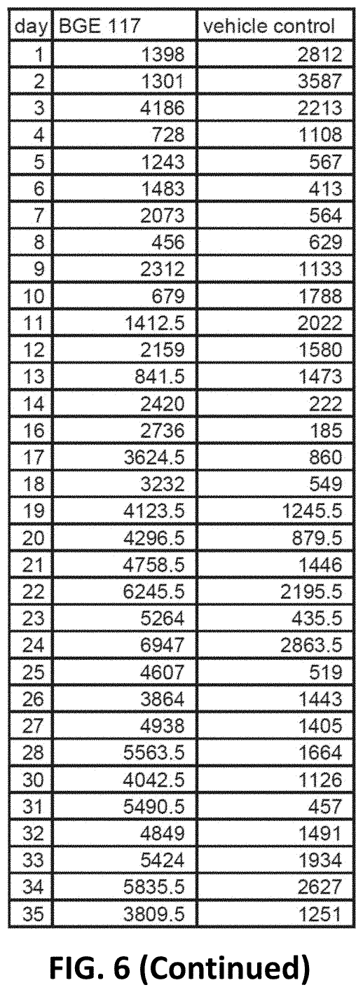

The mice were observed for 35 days after beginning to receive active compound (BGE-117) or vehicle, during which time they received daily gavage of active compound (BGE-117) or vehicle. The animals were housed with access to voluntary running wheels that wirelessly transmit running data to a computer for analysis. The outcome of interest was the median number of daily wheel rotations generated by mice in each group (n=9 per group), and this is shown as the dots in FIG. 6 (one median number of rotations per day per group) and also as the numbers on the right. A best-fit line (generated using LOESS smoothing) was generated for each group, and is shown in FIG. 6 along with the 95% confidence interval for the best-fit line. The p-value shown was generated by calculating the daily difference between the medians in the BGE-117 vs. vehicle groups.

Assessments were done to determine whether the daily differences show a clear directional trend. The formal test involved calculating a Spearman correlation coefficient between these daily differences and the day number (e.g. days 1, 2, 3, etc. of the experiment) and testing the null hypothesis that this correlation coefficient equals 0.

The first day of the study (Study Day 1) started with Animal Acclimation, followed by the BGE-117 treatment start date on Study Day 33 (Phase Day 1). The study concluded on Study Day 67. Activity wheel monitoring started on Study Day 33 (Phase Day 1) and ended on Study Day 67 (Phase Day 35). The total duration of BGE-117 treatment and activity wheel monitoring was 35 days. For the frailty portion of the study, mice were assessed using an activity monitoring wheel which was monitored passively with a computer monitoring system.

Animal welfare was monitored using body weight, clinical evaluation, and body composition score. Animals were evaluated per IACUC protocol and euthanized when criteria were met for euthanasia.

As shown in Table 1, the study included 27-month-old mice from strain C57BL/6. It is known that mice ranging from 18-24 months of age correlate with humans ranging from 56-69 years of age, with mice older than 24 months correlating with humans beyond 69 years old (Flurkey, Currer, and Harrison, 2007, “The mouse in biomedical research” in James G. Fox (ed.), American College of Laboratory Animal Medicine series, Elsevier, A P: Amsterdam; Boston). The latter age range meets the definition of “old,” or “aged” defined as the presence of senescent changes in biomarkers in animals.

TABLE 1
Animals

| Species | Strain | Category | Qty | Age | Source |
|---|---|---|---|---|---|
| Mouse | C57BL/6 | E | 20 ± 5 | ~27 month | Charles River |

The BGE-117 compound for the TA-1 treatment group was formulated in 0.5% carboxymethylcellulose (CMC) and 0.5% Tween 80 and constituted at a concentration of 1 mg/ml. The test article for the Control group included 0.5% CMC and 0.5% Tween 80, acting as a vehicle control. Mice were treated with BGE-117 at a concentration of 1 mg/ml at a volume of 300 μl/mouse (0.3 ml/mouse), for 0.3 mg/dose (TA-1); control group mice (TA-2) were administered a vehicle control at a dose volume of 0.3 ml/mouse. Administration was oral, once per day.

TABLE 2
Test Articles (TA)

| | | | | Dose |
|---|---|---|---|---|
| | Number of | Dose Regimen and | Dose | Concen- |
| Cohort | Animals | Route | Volume | tration |
| TA-1 | 30 ± 0 | Per Os (PO) -- Once | 0.3 ml/mouse | 1 mg/ml |
| (BGE-117, | | Daily (QD) | | |
| 1 mg/ml) | | | | |
| TA-2 | 30 ± 0 | Per Os (PO) -- Once | 0.3 ml/mouse | 0 mg/ml |
| (vehicle | | Daily (QD) | | |
| control) | | | | |

Treatment groups are shown in Table 3.

TABLE 3
Experimental Arm/Group

| Group Number | Number of Animals | Test Article | Animal Age |
|---|---|---|---|
| 1 | 10 ± 0 | TA-1 | 27 month |
| 2 | 10 ± 0 | TA-2 | 27 month |

The study parameters for Groups 1-2 are provided in Table 4. The study parameters for mice in Groups 1-2 included animal acclimation, animal welfare, such as checking the weight of the animal, clinical examination, administering the treatment, activity monitoring, and blood collection, on the particular Study Days and/or Phase Days.

TABLE 4
Procedure Schedule

| | | |
|---|---|---|
| Phase Day (PD)/Study Day (SD) | Experimental Arm/Group | Description |

Acclimation

| | | |
|---|---|---|
| SD 1 | Acclimation: | Weigh mice |
| | Groups 1-4 | Clinical Examination |
| | | Single house in Mouse Cages with |
| | | Activity Monitoring Wheel |
| | | Exclude Nestlets, Innodome, |
| | | Innowheel |
| SD 28 | Active: | Weigh mice |
| | All Groups | Clinical Examination |
| | | Grid Hang acclimation |
| | | 1 trial |
| SD 32 | Active: | Grid Hang Test |
| | All Groups | 1 trial |

Treatment

| | | |
|---|---|---|
| PD 1/SD 33 | Active: | Randomize animals with consideration |
| | All Groups | of behavior tests, Smart Wheel data and |
| | | Body Weights. |
| | | Weigh mice |
| | | Clinical Examination |
| | | Submental Blood Collection |
| | | 100 μL |
| | | i. Hemoglobin (Hb) |
| | | ii. Erythropoietin (EPO) |
| | | PO Dose: 0.3 ml |
| | | Ear Hole Punch |
| | | 1 mm |
| | | Activity Monitoring Wheel: |
| | | Begin monitoring 24 hour activity |
| | | monitoring wheel |
| PD 2-13/SD 34-45 | Active: | Clinical Examination |
| | All Groups | Activity Monitoring Wheel: |
| | | Continue activity monitoring |
| | | PO Dose: 0.3 ml |
| PD 14/SD 46 | Active: | Weigh mice |
| | All Groups | Clinical Examination |
| | | PO Dose: 0.3 ml |
| | | Activity Monitoring Wheel: |
| | | Continue activity monitoring |
| | | Submental Blood |
| | | Collection |
| | | 100 μL |
| | | i. Hb |
| | | EPO |
| PD 15-30/SD 47-62 | Active: | Clinical Examination |
| | All Groups | Activity Monitoring Wheel: |
| | | Continue activity monitoring |
| | | PO Dose: 0.3 ml |
| PD31/SD 63 | Active: | Weigh mice |
| | All Groups | Clinical Examination |
| | | Grid Hang acclimation |
| | | 1 trial |
| | | Activity Monitoring |
| | | Wheel: |
| | | Continue activity |
| | | monitoring |
| | | PO Dose: 0.3 ml |
| PD32-33/SD 64-65 | Active: | Clinical Examination |
| | All groups | Activity Monitoring Wheel: |
| | | Continue activity monitoring |
| | | PO Dose: 0.3 ml |
| PD34/SD 66 | Active: | Grid Hang Test |
| | All Groups | 1 trial |
| | | Activity Monitoring Wheel: |
| | | Continue activity monitoring |
| | | PO Dose: 0.3 ml |
| PD 35/SD 67 | Active: | Weigh mice |
| | All Groups | Clinical Examination |
| | | PO Dose: 0.3 ml |
| | | Activity Monitoring Wheel: |
| | | End activity monitoring |
| | | Submental Blood Collection |
| | | 100 μL |
| | | i. Hb |
| | | ii. EPO |
| | | Observe Ear Punch |
| | | Score |
| | | Open greater than 0.25 mm |
| | | Open less than 0.25 mm |
| | | Closed |
| | Humane Endpoint: | If animal meets humane endpoint: |
| | (criteria for | Notified study director and take |
| | euthanasia): | note of time and cage number and |
| | 1. Moribund | euthanize animal as appropriate. |
| | a. Indicated by: | |
| | BCS = 1 for 48 | |
| | consecutive hours | |
| | OR 25% weight loss | |
| | OR severely hunched | |
| | posture with | |
| | squinting eyes OR | |
| | inability to self-right | |
| | 2. Agonal breathing/ | |
| | cyanosis | |
| | 3. Inability to eat or | |
| | drink | |
| | 4. Severe diarrhea | |
| | 5. Severe bleeding | |
| | from any orifice | |
| | 6. Severe | |
| | convulsions from which the | |
| | animal does not recover (e.g. | |
| | status epilepticus) | |
| | 7. Self-induced trauma (e.g. | |
| | autotomy) | |
| | Animal Found Dead: | If an animal is found dead: |
| | | Notified study director and take |
| | | note of time and cage number |

### Activity Monitoring Wheel Test

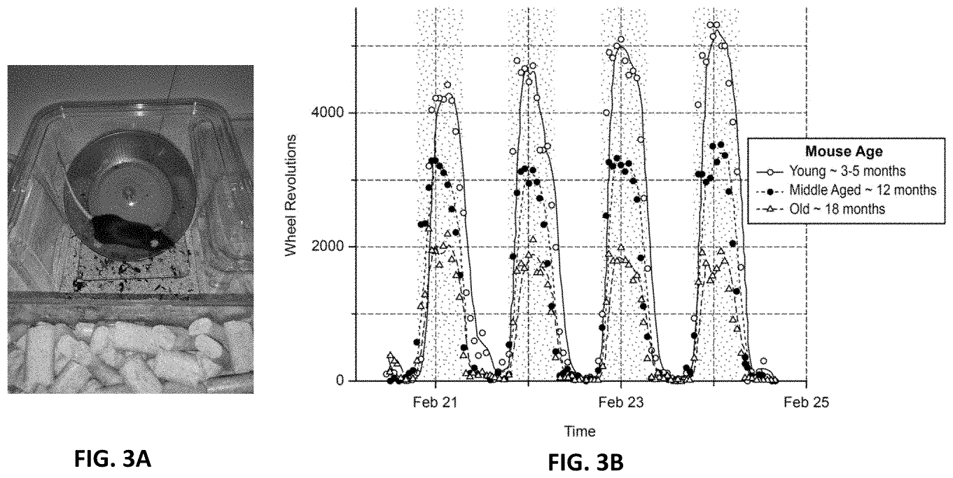

The activity monitoring wheel is a running disk that monitors rotations. See FIG. 3A. The wheel is capable of monitoring voluntary wheel running 24 hours a day. Activity was monitored passively and wirelessly with a computer monitoring system. Running wheel activity levels were monitored daily. The wheel data were daily median rotations in each group (BGE-117 treated vs. controls).

The running disk, as shown in FIG. 3A, included a wheel that was present in each mouse cage (each cage has one wheel and one mouse) for the entirety of the study. The wheels were electronically monitored and the number of rotations of the wheel per minute was recorded continuously throughout the entire study. These data are summarized in FIG. 6 as the total number of rotations per mouse per day. FIG. 6 shows the median of these rotations per day per experimental group.

### Blood Collection

Using a 4 mm lancet, approximately 150 microliters of whole blood was collected from the submental vein, utilizing the submandibular vein as back-up. Blood was collected using 500 microliter Serum Separator tubes and stored at room temperature for 45 minutes or until clotted. Blood was centrifuged at 4000 RPM for 10 minutes in at 4° C. If the serum was not analyzed immediately, the serum was apportioned into 0.5 ml aliquots, stored, and transported at −80° C.

### Hemoglobin (Hb) Assay Procedures

The following procedure was performed for the hemoglobin assay:
1. Mouse blood was collected in Li-heparin tubes.
2. Blood was briefly vortexed and 2 μl of whole blood was transferred into a 96-well clear plate containing 18 μl of distilled water.
3. 160 μl sample buffer was added to the 96-well clear plate from the Hemoglobin Assay Kit (ab234046).
4. 20 μl 1N NaOH was added to blood samples to the diluted sample in each well.
5. The standard curve was established with included hemoglobin standard (ab234046) as per the manufacturer's instruction.
6. The reaction was incubated at room temperature for 8-15 minutes. Read absorbance at 575 nm in end point mode.
7. Hb concentration was calculated as recommended by the assay kit.

### Study Results

As shown in FIG. 6, BGE-117 treated mice demonstrated significantly increased activity as compared to controls, as shown from the activity wheel test.

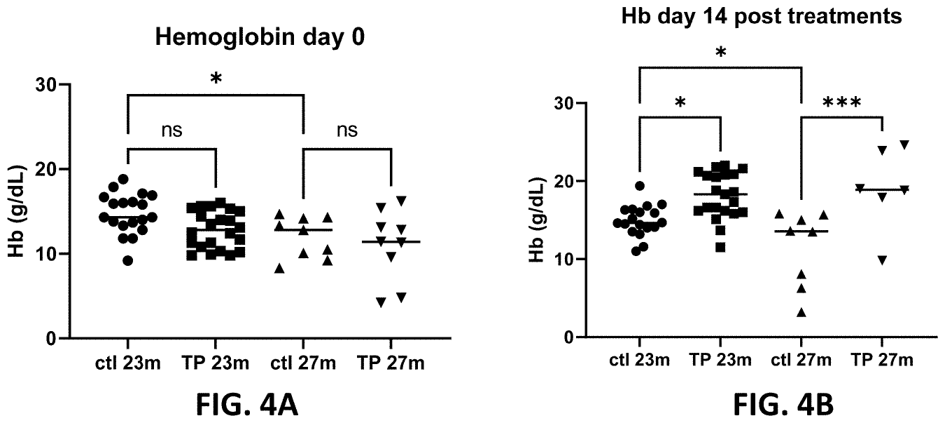

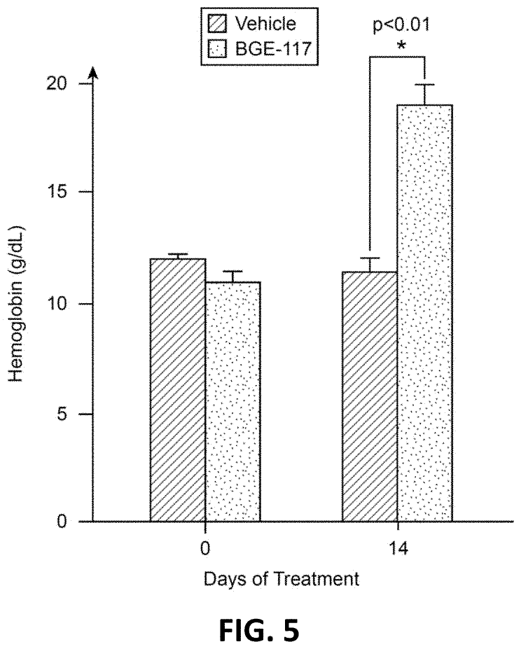

In addition, 27-month old mice treated with BGE-117 showed improved hemoglobin levels, as summarized in FIGS. 4 and 5 and Table 5 below.

TABLE 5
Hemoglobin levels in aged mice

| Week | Group | Mean (g/dl) | Standard error |
|---|---|---|---|
| 0 | BGE-117 | 10.97778 | 0.466362 |
| 2 | BGE-117 | 19 | 0.885877 |
| 0 | Placebo | 11.93333 | 0.269602 |
| 2 | Placebo | 11.4 | 0.605407 |

Thus, the compound BGE-117 can counteract age-related frailty, increasing physical performance, and can counter anemia in otherwise healthy old (aged) mice.

### 5.4.4. Example 4

### BGE-117 is Effective to Treat Unexplained Anemia of the Elderly and Anemia of Inflammation in Aged Mice

BGE-117 (BGE-117) is a competitive HIF prolyl hydroxylase (PHD) 1/2/3 pan-inhibitor which stabilizes HIF-1α, leading to increased EPO production in both mice and humans. Although BGE-117 is known to inhibit HIF-PH and increase EPO production in patients with chronic kidney disease and in normal healthy human volunteers (Shinfuku et al., Am. J. Nephrol. 48(3):157-164 (2018)), and has been shown to increase hemoglobin levels in 5/6 nephrectomized rats (Kato et al., J. Pharmacol. Exp. Ther. 371:675-683 (2019), the effect of BGE-117 on anemia of inflammation is still unknown. In addition, the effect of BGE-117 on unexplained anemia of aging, and anemia of inflammation in old aged subjects, is still unknown.

In Example 1, we demonstrated that BGE-117 was able to increase hemoglobin levels in old, but otherwise healthy, mice. The goal of this study was to assess the effect of BGE-117 on unexplained anemia of aging and anemia of inflammation in aged mice having higher circulating levels of inflammatory cytokines (130% or higher of the median values).

### Study Design

The study included 23-month-old mice (n=39) (23-months old at the start of the experiment, and 24 months old at the end since the experiment lasted 35 days) and 27-month old mice (n=14) (27-months old at the start of the experiment, and 28 months old at the end since the experiment lasted 35 days) from strain C57BL/6 that were administered BGE-117 or vehicle once daily for a duration of 14 days.

### Materials and Methods

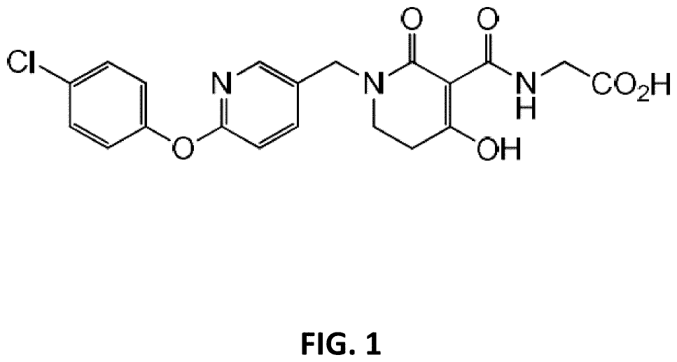

Reagents: 2-[[1-[[6-(4-Chlorophenoxy)pyridin-3-yl]methyl]-4-hydroxy-6-oxo-2,3-dihydropyridine-5-carbonyl]amino]acetic acid (BGE-117, FIG. 1) was synthesized according to a previously described method and Example 1.

Animal Welfare: Animal welfare was monitored as described in Example 1.

### Measurement of Hemoglobin and Inflammatory Markers

In order to determine if 23-month old and 27-month old C57BL/6 mice spontaneously developed anemia, and to assess the contribution of inflammation to spontaneous anemia, mice were tested for hemoglobin levels and inflammatory markers TNFα and IL-6.

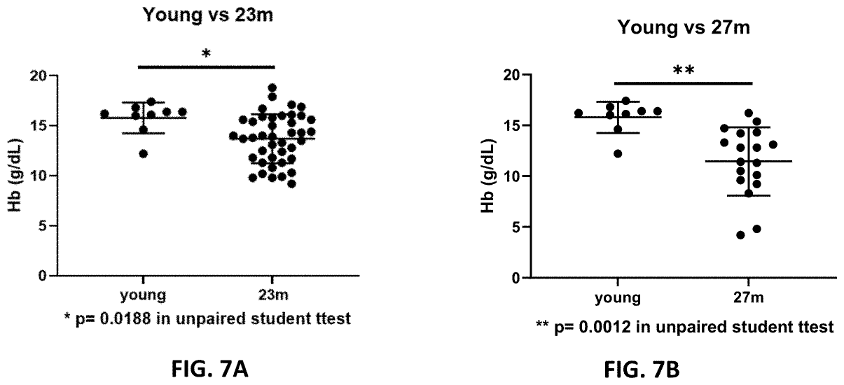

Hemoglobin levels were measured in 23-month old mice at 23 months. 23-month-old mice were found to have statistically significantly lower hemoglobin concentration as compared to hemoglobin concentration of young (3-6 month old) mice, as shown in FIG. 7A (p=0.0188). Hemoglobin levels were measured in 27-month old mice at 27 months. 27-month-old mice were found to have statistically significantly lower hemoglobin concentration as compared to hemoglobin concentration of young (3-6 month old) mice, as shown in FIG. 7B (p=0.0012).

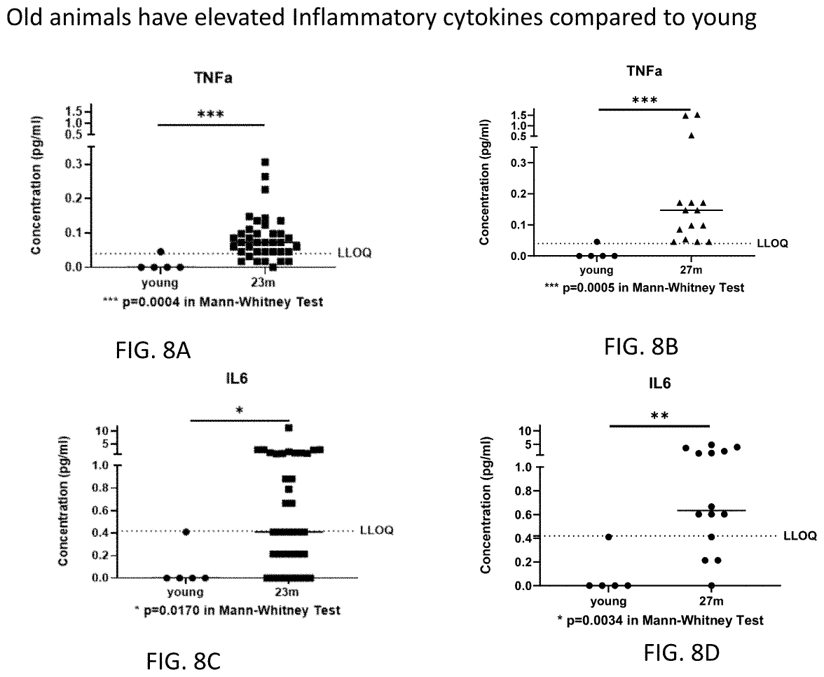

Inflammatory cytokine levels were measured in 23-month old mice at 23 months (±2 weeks). 23-month old mice were found to have statistically significantly elevated levels of both TNFα (p=0.0004) and IL-6 (p=0.0170) in comparison to normal young animals (3-6 months old) as shown in FIGS. 8A and 8C. Inflammatory cytokine levels were also measured in 27-month old mice at 27 months (±2 weeks). 27-month old mice were found to have statistically significantly elevated levels of both TNFα (p=0.0005) and IL-6 (p=0.0034) in comparison to normal young animals (3-6 months old) as shown in FIGS. 8B and 8D.

These findings demonstrate natural development of anemia of aging in otherwise healthy mice, as evidenced by low hemoglobin levels in 23-month old and 27-month old mice compared to 3-6 month old young mice, and demonstrate that the spontaneous anemia of aging was accompanied by inflammation, as evidenced by elevated IL-6 and TNFα levels, in 23-month old and 27-month old mice in comparison to normal young animals.

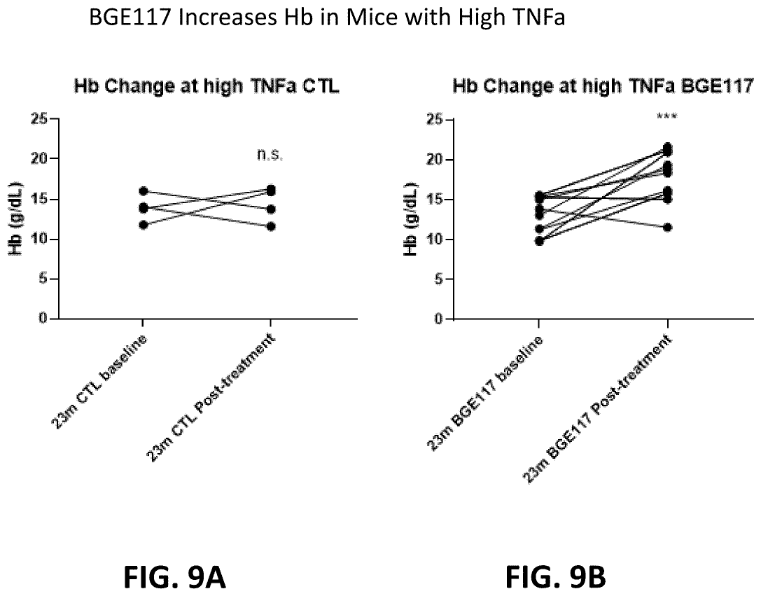

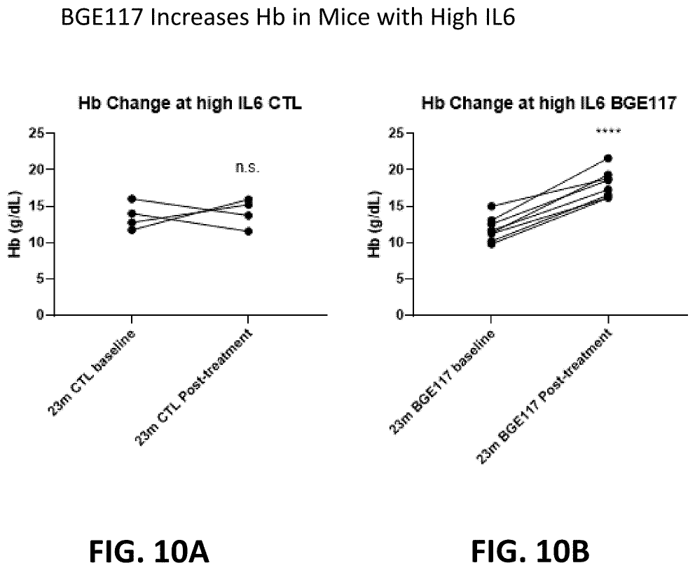

Once the 23 month old mice developed anemia of inflammation at 23-months (±2 weeks), mice were randomly divided into two groups: Anemia of inflammation vehicle-treated Control group 1 (“23-month Control Group” (CG1)) (n=18) and Anemia of inflammation BGE-117-treated group (“BGE-117 23-month Test Group” (BGE-117 TG1)) (n=21), while ensuring that the mean hemoglobin levels and inflammatory cytokine levels remained balanced among the groups. The mice in the two “Anemia of inflammation” pre-treatment (baseline) groups had high TNFα and/or IL-6 as indicated in FIGS. 9-10.

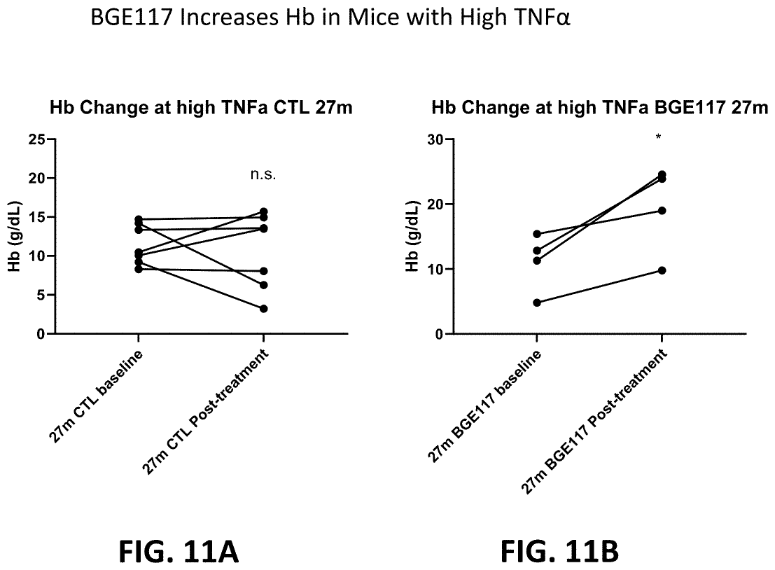

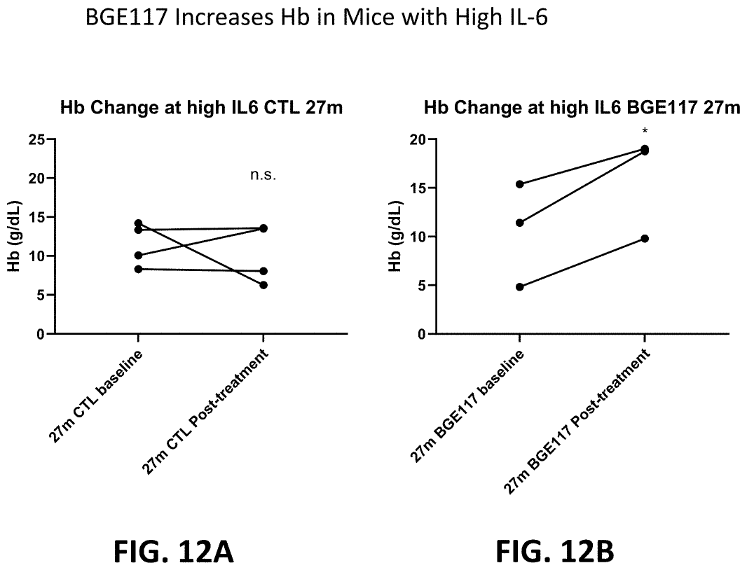

Once the 27-month old mice developed anemia of inflammation at 27-months (±2 weeks), mice were randomly divided into two groups: Anemia of inflammation vehicle-treated Control group (“27-month Control Group” (CG2)) (n=8), Anemia of inflammation BGE-117-treated group (“BGE-117 27-month Test Group” (BGE-117 TG2)) (n=6), while ensuring that the mean hemoglobin levels remained balanced among the groups. The mice in the two “Anemia of inflammation” pre-treatment (baseline) groups had high (130% or higher of the median values) TNFα and/or IL-6 as indicated in FIGS. 11-12.

As shown in FIGS. 8A-8D, 23-month old mice and 27-month old mice had elevated levels of IL-6 and TNFα relative to 3-6 month old young mice.

The 23-month old and 27-month old Test Groups BGE-117 TG1 and BGE-117 TG2 were administered 10 mg/kg of BGE-117 once daily for 5 weeks by oral gavage in 0.5% carboxymethylcellulose (CMC) and 0.5% Tween 80. The 23-month old and 27-month old Control Groups CG1 and CG2 were administered vehicle of 0.5% carboxymethylcellulose (CMC) and 0.5% Tween 80 by oral gavage on the same schedule.

Blood was collected from the submandibular vein at the start of the study immediately before drug administration, and after 14 days of drug administration. The blood samples was mixed with EDTA and analyzed using the Hemoglobin assay kit from Abcam (ab234046) with modification. The signal was read on Molecular Device SpectraMax 340PC-384. The inflammatory markers were measured by Luminex Mouse Magnetic Assay (5-PLEX, LXSAMSM-05 by R&D Systems) on Luminex 200. At day 35 post drug initiation the mice were anesthetized with isoflurane and blood samples were collected from abdominal vein.

The blood samples are mixed with EDTA and the samples were then centrifuged (4° C., 2130×g for 10 min) to obtain plasma.

### Results

Anemia developed naturally in 23-month-old and 27-month old C57BL/6 mice, mimicking unexplained anemia of aging in humans. The 23-month old and 27-month old anemic mice had elevated levels of pro-inflammatory cytokines TNFα and IL-6 relative to young 3-6 month old mice (FIGS. 8A-8D) suggesting that the underlying etiology of the anemia was anemia of inflammation.

Administration of BGE-117 in the 23-month old and 27-month old Test Groups significantly increased Hb levels (FIGS. 9B and 10B for 23-month old mice, and FIGS. 11B and 12B for 27 month-old mice), as compared to Hb levels of the 23-month old and 27-month old Control Groups (FIGS. 9A and 10A for 23-month old mice, FIGS. 11A and 12A for 27-month old mice) administered vehicle alone.

Thus, administration of BGE-117 was effective to reduce anemia of inflammation, despite the presence of elevated TNFα and IL-6, which is known to increase hepcidin levels and cause sequestration of iron within cells and functional anemia. Moreover, these effects were observed in 23-month-old mice and 27-month old mice for BGE-117, which correspond to elderly human subjects.

### 5.4.5. Example 5

### Roxadustat is Effective to Treat Unexplained Anemia of the Elderly and Anemia of Inflammation in Old Mice

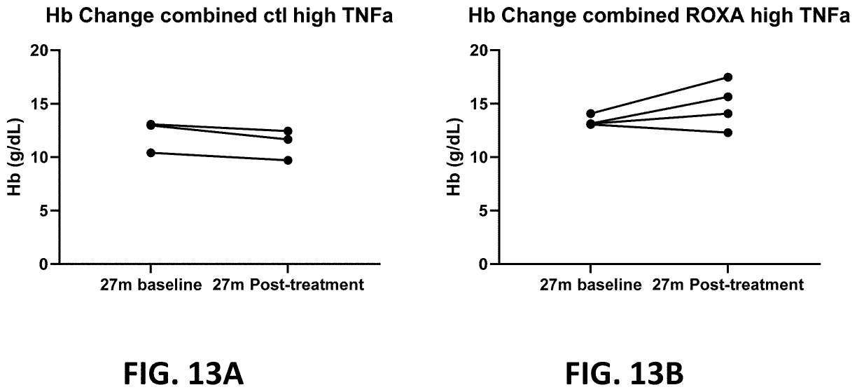

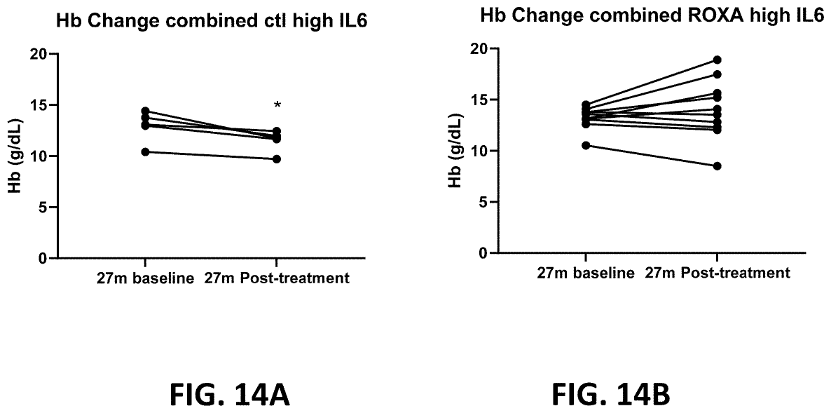

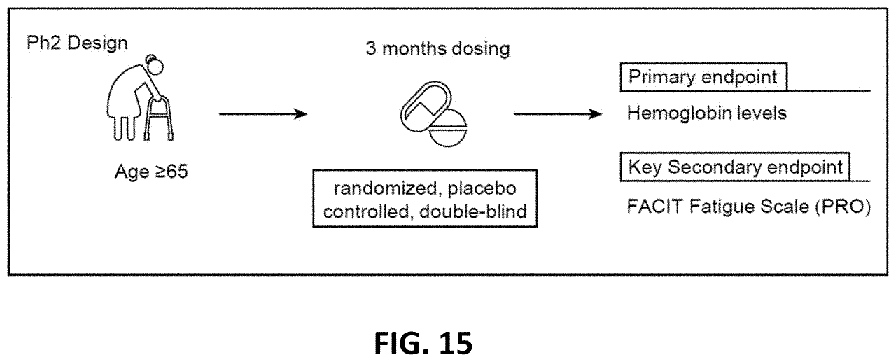

The goal of this study was to assess the effect of Roxadustat, a HIF-PH inhibitor, on unexplained anemia of aging and anemia of inflammation in old mice with higher inflammatory cytokines (130% or higher of the median values) as shown in pretreatment (baseline) groups of FIGS. 13-14.

### Study Design

The study included 27-month old mice (n=32) (27-months old at the start of the experiment, and 28 months old at the end since the experiment lasted 35 days) from strain C57BL/6 that were administered roxadustat or vehicle once daily for a duration of 14 days.

### Materials and Methods

Reagents: Roxadustat, at a concentration of 4 mg/ml, was prepared by slowing dropping powder (MedKoo Biosciences, Inc CAT #: 317133) in stirred aqueous solution of 0.5% carboxymethyl cellulose and 0.5% Tween-80. The dosing suspension was continually stirred for 2 hours, aliquoted, and stored at −20° C. till use. The dosing suspension, which had been thawed at room temperature and vortexed immediately before each day's dose.

Animal Welfare: Animal welfare was monitored as described in Example 1.

### Measurement of Hemoglobin and Inflammatory Markers

In order to determine if 27-month old C57BL/6 mice spontaneously developed anemia, and to assess the contribution of inflammation to spontaneous anemia, mice were tested for hemoglobin levels and inflammatory markers TNFα and IL-6.

Hemoglobin levels were measured in 27-month old mice at 27 months. 27-month-old mice were found to have low hemoglobin concentration as compared to hemoglobin concentration of young (3-6 month old) mice, as shown in FIG. 7B.

Inflammatory cytokine levels were also measured in 27-month old mice at 27 months (±2 weeks). 27-month old mice were found to have elevated levels of both TNFα and IL-6 in comparison to normal young animals (3-6 months old) as shown in FIGS. 8B and 8D.

These findings demonstrate natural development of anemia of aging in otherwise healthy mice, as evidenced by low hemoglobin levels in 27-month old mice compared to 3-6 month old young mice, and that the spontaneous anemia of aging was accompanied by inflammation, as evidenced by elevated IL-6 and TNFα levels in 27-month old mice in comparison to normal young animals.

Once the 27-month old mice developed anemia of inflammation at 27-months (±2 weeks), mice were randomly divided into two groups: Anemia of inflammation vehicle-treated Control group (“27-month Control Group” (CG2)) (n=9) and Anemia of inflammation Roxadustat-treated group (“27-month old Roxa Test Group” (Roxa TG1)) (n=23), while ensuring that the mean hemoglobin levels remained balanced among the groups. The mice in the two “Anemia of inflammation” pre-treatment (baseline) groups had high (130% or higher of the median values) TNFα and/or IL-6 as indicated in FIGS. 13-14.

The 27-month old Test Group Roxa TG1 was administered 40 mg/kg of Roxadustat once daily (10 ml/kg dosing suspension) for 2 weeks by oral gavage.

Blood was collected from the submandibular vein at the start of the study immediately before drug administration, and after 14 days of drug administration. The blood samples were mixed with EDTA and analyzed using the Hemoglobin assay kit from Abcam (ab234046) with modification. The signal was read on Molecular Device SpectraMax 340PC-384. The inflammatory markers were measured by Luminex Mouse Magnetic Assay (5-PLEX, LXSAMSM-05 by R&D Systems) on Luminex 200. At day 35 post drug initiation the mice were anesthetized with isoflurane and blood samples were collected from abdominal vein.

The blood samples are mixed with EDTA and the samples were then centrifuged (4° C., 2130×g for 10 min) to obtain plasma.

### Results

Anemia developed naturally in 27-month old C57BL/6 mice, mimicking unexplained anemia of aging in humans. The 27-month old anemic mice had elevated levels of pro-inflammatory cytokines TNFα and IL-6 relative to young 3-6 month old mice (FIGS. 8A-8D) suggesting that the underlying etiology of the anemia was anemia of inflammation.

Administration of Roxadustat in the 27-month old Test Groups increased Hb levels (FIG. 13B for 27-month old mice with elevated levels of TNFα, and FIG. 14B for elevated levels of IL-6), as compared to Hb levels of the 27-month control group (FIG. 13A and FIG. 14A) administered with vehicle alone. Although not statistically significant, administration of 40 mg/kg of Roxadustat was effective to improve hemoglobin levels in 27-month old mice with elevated levels of pro-inflammatory cytokines TNFα and IL-6, whereas untreated animals showed a significant decline in hemoglobin over the study period (FIG. 14A).

Thus, administration of Roxadustat was effective to reduce anemia of inflammation, despite the presence of elevated TNFα and IL-6. Moreover, these effects were observed in 27-month old mice for Roxadustat, which correspond to aged human subjects.

### 5.4.6. Example 6

### BGE-117 Improves Activity Levels and Hemoglobin Levels in a Phase 2 Study

Study Title: A Phase 2a, 12-Week, Randomized, Double-Blind, Placebo-Controlled, Multi-Center Study to Evaluate Efficacy and Safety of BGE-117 in the Treatment of Unexplained Anemia of Aging (UAA)

### Study Design Summary

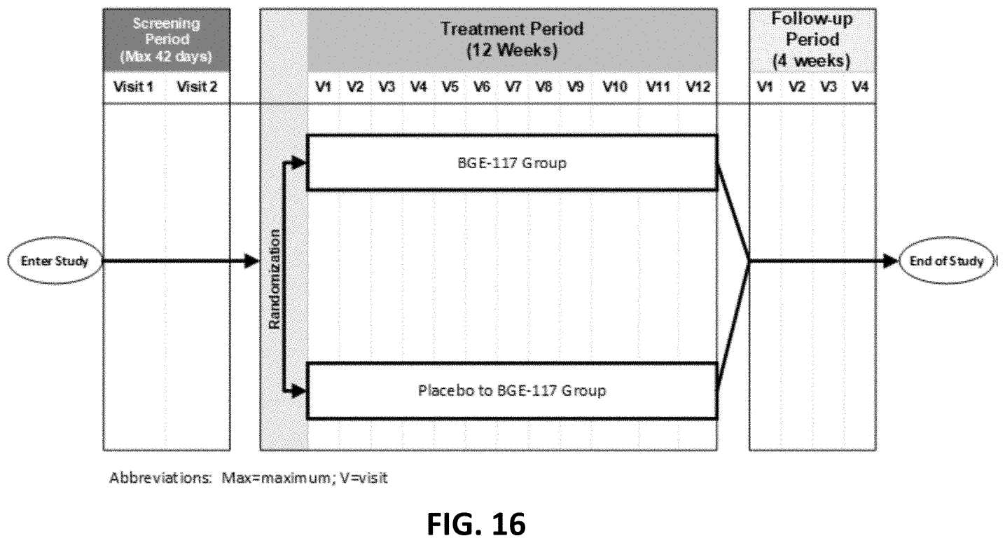

This is a Phase II, randomized, double-blind, placebo-controlled multi-center study to compare the safety of BGE-117 when administered in patients over 65 years of age for 12 weeks. The study consists of 3 periods: Screening, Treatment, and Follow-up. FIG. 16 provides a summary of the study design.

The study size for this study is 160 evaluable subjects (80 subjects randomized to BGE-117 and 80 subjects randomized to placebo). The subject's maximum duration of participation is approximately 154 days. This includes 6 weeks for the Screening Period, 12 weeks for the Treatment Period, and 4 weeks for the Follow-up Period.

When the subject enters the Screening Period, subject eligibility is confirmed at a minimum of 2 screening visits during the Screening Period. Subjects who satisfied all eligibility criteria during the Screening Period (Day −42 to Day −1) may be randomized at the beginning of Visit 3 (Day 1).

### Objectives

Primary Objectives:
To evaluate the efficacy of BGE-117 in the treatment of UAA.

Secondary Objectives:
To evaluate the safety and tolerability of BGE-117 in the treatment of UAA
To evaluate the overall Hb control of BGE-117 in the treatment of UAA
To evaluate the appropriateness of the starting dose of BGE-117 in the treatment of UAA
To characterize the population PK of BGE-117 in the treatment of UAA
To evaluate the use clinical outcome assessments (scales) in the treatment of UAA

Exploratory
To explore nonspecific proteomics, metabolomics and transcriptomics in peripheral blood of elderly subjects with UAA in response to, before, during, and after BGE-117 treatment
To explore activity levels and sleep quality of elderly subjects with UAA in response to, before, during, and after BGE-117 treatment

### Entry Criteria

Inclusion Criteria
1. Ability to voluntarily provide written, signed, and dated informed consent to participate in the study
2. An understanding, ability, and willingness to fully comply with study procedures and restrictions
3. Is 65 years of age or older at the time of the first screening visit (this inclusion criterion is only assessed at the first screening visit)
4. UAA defined as:
   Average Hb≥9.0 g/dL but ≤11.0 g/dL. Two laboratory measures are obtained, at least 7 days apart, during the Screening Period. The Hb results are to be consistent with the following:
      At least one Hb measurement ≥9.0 g/dL but ≤11.0 g/dL
      Both Hb measurements are within 0.5 g of each other
   Thyroid-stimulating hormone (TSH) ≥0.1 mcU/mL but ≤10.0 mcU/mL
   Serum iron ≥60 μg/dL, transferrin saturation ≥15.0% and serum ferritin≥30.0 ng/mL
   Mean corpuscular volume (MCV) ≤100.0 fL with no other cause and/or additional cytopenias (platelet count <130.0 K/μl, white blood cell [WBC] count <4 K/μl)
   Folic acid and vitamin B12 levels greater than the lower limit of normal range
5. eGFR as measured by Modification of Diet in Renal Disease (MDRD) ≥30.0 mL/m/1.73 m²
6. The FACIT-Fatigue Scale with a score ≤35.0
7. Weight at the first screening visit is ≥40.0 kg

Exclusion Criteria
1. History or diagnosis of any of the following:
   Anemia due to pernicious anemia, thalassemia, sickle cell anemia, sickle trait, or myelodysplastic syndromes
   Clinically diagnosed chronic inflammatory disease that could impact erythropoiesis (e.g., systemic lupus erythematosus, rheumatoid arthritis, celiac disease) even if it is currently in remission
   Bone-marrow hypoplasia or pure red cell aplasia
   Androgen deprivation therapy within the prior 12 months or radiation treatment for prostate cancer
   Intravenous iron within 12 weeks before the first screening visit or during the Screening Period
      Note: Oral iron is acceptable. However, the same dose regimen must be used throughout the Screening and the Treatment Period.
   Myocardial infarction, acute coronary syndrome, stroke, transient ischemic attack, or pro-thrombotic arrhythmia or condition (e.g., untreated atrial fibrillation) within 6 months before screening or during the Screening Period
   Cancer diagnosis with active disease (i.e. active malignancy), or are receiving active treatment within 12 weeks before the Screening Period (squamous cell or basal cell carcinoma of the skin are excluded from this criterion)
2. Suspected or hematologic malignancy defined as:
   Previous diagnosis with confirmation by bone marrow examination
   MCV ≥100 fL with no other cause, and/or additional cytopenias (platelet count <130 K/μl, WBC count <4 K/μl)
   Previously observed dysplasia on blood smear or otherwise unexplained monocytosis
3. History of hypertension including:
   Difficult-to-control hypertension (unless approved by the investigator and the Medical Monitor)
   Malignant hypertension (unless approved by the investigator and the Medical Monitor)
   Systolic blood pressure≥160 mmHg or diastolic blood pressure≥95 mmHg (confirmed by repeated measurement) within 2 weeks before randomization.
      Note:
         Subjects on hypertension medication should have been on a stable dose and medication for at least 8 weeks before randomization
         Subjects may be rescreened once blood pressure is controlled
4. Current evidence of gastrointestinal bleeding within 12 weeks before the first screening visit, as judged by the investigator
5. Class III heart failure, as defined by the New York Heart Association functional classification system
6. QT interval corrected for heart rate using Fridericia's formula (QTcF) >500 msec or QTcF >530 msec in subjects with bundle branch block
   Note: This evaluation is to be done at only the first screening visit; ECG and corresponding intervals and overall interpretation can be mechanically or manually read by an appropriately designated and trained personnel.
7. ALT and AST ≥3× the upper limit of normal (ULN)
8. Bilirubin >1.5×ULN (isolated bilirubin >1.5×ULN is acceptable if bilirubin is fractionated and direct bilirubin is <35%)
   Note: Bilirubin increases associated with Gilbert's syndrome are permitted.
9. A reported average intake of alcohol of ≥80 g/day (i.e., equivalent of 6 cans of beer or 5 shots of hard liquor)
10. Hb increase to the target range (12.0-13.0 g/dL) would pose an unacceptable medical risk to the subject, as judged by the investigator
11. History of severe allergic or anaphylactic reactions or hypersensitivity to excipients in the IP
12. Use of an investigational agent within 30 days or 5 half-lives of the investigational agent; whichever is longer
13. Prior randomization in BGE-117-201
14. Any current unstable medical condition that the investigator considers would put the subject at unacceptable risk, affect study compliance, or prevent the understanding of the study's objectives or investigational procedures or possible consequences. This includes:
   Current, unstable active liver or biliary disease (generally defined by the onset of ascites, encephalopathy, coagulopathy, hypoalbuminemia, esophageal/gastric varices, persistent jaundice, or cirrhosis)
      Note: Stable liver disease (including asymptomatic gallstones, asymptomatic chronic hepatitis B/C, or Gilbert's syndrome) is acceptable if the subject otherwise meets entry criteria and the investigator and Sponsor approve entry into the study.
15. Current or relevant history of psychiatric illness that may require treatment or make the subject unlikely to complete the study

### Methodology

Safety: Safety endpoints are summarized by treatment group and visit. Descriptive statistics, including potential clinical importance, change from baseline, and out of range values, are calculated for quantitative safety data.

Pharmacokinetics: Blood samples for PK analysis are drawn at the times described in the protocol. Attempts are made to develop a Population PK model based on the data. The results of the Population PK analysis are presented in a separate report.

Proteomics, metabolomics and transcriptomics: Peripheral blood samples are obtained at specified times for possible proteomic and transcriptomic assessments.

Activity levels and sleep quality: Patients are provided with wearable activity monitors which will continuously capture accelerometry data allowing for subsequent calculation of activity levels and sleep quality.

### Administration of Investigational Product(s)

### Dosing

Subjects are advised to take IP with water once a day, recommended that the subject take the study medication 1 hour before their morning meal, starting on Visit 3 (Day 1), for 84 consecutive days, through Visit 15 (Day 85). Capsules are taken whole, not crushed, chewed, or cut.

This study is a double-blind, placebo-controlled study. Subjects are randomized to receive either of the following:
BGE-117: a hypoxia inducible factor-prolyl hydroxylase (HIF-PHD) inhibitor in 4 mg and 12 mg capsules; or
Placebo: matching capsule without active drug
The starting dose and maximum dose for BGE-117/placebo is based on a subject's screening renal function results:
The starting dose for subjects with an estimated glomerular filtration rate (eGFR) ≥60 mL/min/1.73 m2 is 12 mg/placebo. The maximum dose is 24 mg/placebo.
The starting dose for subjects with an eGFR between ≥30 and <60 mL/min/1.73 m²is 4 mg/placebo. The maximum dose is 16 mg/placebo.

### Treatment Period

The treatment period has 3 dosing periods, described in Table 6 below:

| | | Comment |
|---|---|---|
| | | Note: dose reductions are allowed |
| Dosing | Visit Numbers | for safety or tolerability concerns |
| Periods | (Study Days) | at any time during the study |
| Initiation | Visits 3 and 4 | The subject starts taking IP according |
| | (Days 1-14) | to the dosing algorithm. |
| Adjustment | Visits 5, 6, 7, 8, 9, | The IP dose is adjusted so that the |
| | 10, & 11 | subject's Hb is within the target |
| | (Days 15-57) | range. |
| Maintenance | Visits 12, 13, 14, & | Subjects remain at the same dose |
| | 15 | until the EOTP. |
| | (Days 58-85) | |

### Dose Adjustment Guidelines

From Visits 5 through 11 (Days 15 through 57), dose adjustment opportunities are scheduled approximately every 14 days to achieve and maintain Hb within the target range. Dose adjustments are based on the local laboratory's results, with the goal of titrating Hb levels to a range of ≥12.0 to ≤13.0 g/dL. In the event of excessively rapid Hb increases or unacceptably high Hb levels or trends, in the investigator's opinion, the dose may be adjusted at any time, even on visits without a dose adjustment review.

The allowable dose levels for subjects with an eGFR ≥60 mL/min/1.73 m2 are 0 (discontinuation), 4, 8, 12, 16, 20, and 24 mg. The maximum dose is 24 mg/placebo.

The allowable dose levels for subjects with an eGFR between ≥30 and <60 mL/min/1.73 m2 are 0 (discontinuation), 4, 8, 12, and 16 mg. The maximum dose is 16 mg/placebo.

A “one-step” escalation or reduction in dose refers to, respectively, an increase or decrease of 4 mg. The only dose increase that is permitted at any one visit is “one step”, i.e., 4 mg. Dose reductions may exceed 4 mg based on the reason for the reduction and the investigator's judgment.

The specific dose adjustment guidelines are as follows:
One-step dose escalations may occur at Visits 5, 7, 9, and 11 (Days 15, 29, 43, and 57) if needed, with no additional dose escalations permitted after Visit 11 (Day 57), if the subject's Hb increase in the previous 2 weeks is ≤0.5 g/dL and the subject has Hb <12.5 g/dL.
No dose escalations are permitted if Hb >12.5 g/dL.

Dose decreases may be made at any time during the study. If a decrease is made at or after Visit 11 (Day 57), the dose may not be subsequently increased again to that same level.

If a subject's Hb exceeds 13.0 g/dL at any time, or if a subject's Hb increased >1.0 g/dL in the previous 2 weeks or >1.5 g/dL in the previous 4 weeks, the dose can be decreased following these guidelines:
A dose of 24 mg is reduced to 12 mg.
A dose of 20 mg is reduced to 8 mg.
A dose of 16 mg is reduced to 8 mg.
A dose of 12 mg is reduced to 4 mg.
A dose of 8 mg is reduced to 4 mg.
A dose of 4 mg is reduced to 0 mg (discontinuation).

If a subject's Hb exceeds 13.5 g/dL at any time, dosing is discontinued, and Hb levels is followed until they have returned to below 13.0 g/dL.

There is a possibility that the investigator is be able to recommend a dose adjustment at the time of a subject's visit due to the local Hb results not being known. Once the investigator had an opportunity to review the Hb result, the investigator consults the subject about the Hb level and the dose to be taken until the next visit. It is anticipated that it may take 24 to 96 hours after the subject's visit and when the subject may start taking the new, adjusted dose.

### Physical Examination (Including Height and Weight)

A complete physical examination is performed at various timepoints by a qualified physician, physician's assistant, or nurse practitioner

The physical examination includes a review of the following body systems:
General appearance
Spine/neck/thyroid
Cardiovascular
Skin
Musculoskeletal
Neurological
Head, eyes, ears, nose, and throat
Respiratory
Abdomen (including liver and kidneys)

Clinically significant abnormalities identified at the screening visit are documented in the subject's source documents and in the Medical History eCRF/CRF. Clinically relevant changes after the initial Screening Period visit are captured on the AE eCRF/CRF page, as deemed by the investigator. Note: height is only collected at the initial examination.

An abbreviated physical examination is performed at various time points, and include:
Height
Weight
Vital signs (blood pressure and pulse rate)
Clinical laboratory tests
Complete blood count:
   Basophils (absolute)
   Eosinophils (absolute)
   Hematocrit
   Hemoglobin
   HbA1C (4 mL) collected only at Visit 3 (Day 1) and EOTP
   Reticulocyte count (absolute)
   Red blood cells
   Total neutrophils (absolute)
   White blood cell count, total and differential

### Clinical Chemistries

Blood samples (6 mL) for serum clinical chemistries are collected. The following parameters are assessed:
Alanine aminotransferase (ALT)
Carbon dioxide
Phosphate
Albumin
Chloride
Potassium
Alkaline phosphatase
Creatinine
Protein
Aspartate aminotransferase (AST)
Gamma-glutamyl transferase (GGT)
Urea nitrogen□
Calcium
Glucose
Uric acid

### Coagulation and D-Dimer

Blood samples (3 mL) for coagulation are collected. The following parameters are assessed:
Prothrombin time (PT)
D-Dimer
Activated partial thromboplastin time (aPTT)
International normalized ratio (INR)

### Erythropoietin

Blood samples (2 mL) for serum EPO levels are collected.

### Hemoglobin Electrophoresis (Hemoglobinopathy Fractionation)

Blood samples (1 mL) for hemoglobin electrophoresis (hemoglobinopathy fractionation) are collected.

### Inflammation Panel

Blood samples (2 mL) for an inflammation panel are collected. The following parameters are assessed:
C-reactive protein (CRP)
Interleukin (IL-6)
Hepcidin

### Iron Panel

Blood samples (2 mL) for a serum iron panel are collected. The following parameters are assessed:
Serum iron
Total iron-binding capacity
Transferrin saturation
Serum transferrin
Serum ferritin

### Folate and Vitamin B12

Blood samples (2 mL) for folate and vitamin B12 are collected. The following parameters are assessed:
Folate (folic acid)
Vitamin B12

### Lipid Panel

Blood samples (2 mL) for non-fasting lipid levels are collected. The following parameters are assessed:
HDL
Cholesterol
Total Triglycerides
Low-density lipoprotein (calculation)

### Fecal Occult Blood Test

Consenting subjects provide a stool specimen collected from one bowel movement or as described in the Laboratory Manual.

### Ophthalmic Examination

An ophthalmic examination is conducted by an appropriately delegated ophthalmologist or optometrist. Each assessment includes a comprehensive eye examination with at least the following components: measurement of best-corrected visual acuity, intraocular pressure, anterior aqueous chamber examination, and a fundoscopic examination. These examinations are used for the assessment of ocular AEs.

### Vascular Doppler Ultrasound for Deep Vein Thrombosis

Vascular Doppler ultrasound is used to scan for the presence of deep vein thrombosis (DVT). DVT scans are performed at various time points by an appropriately delegated employee.

### Pharmacokinetic Collection

Pharmacokinetic blood samples (4 mL) are taken at various time points. The name and address of the bioanalytical laboratory performing the PK assessment for this study are maintained in the investigator's files at each site and in the Trial Master File at the Sponsor.

Actual PK blood sample collection times versus the time of dosing is monitored. The Sponsor expects that the investigator will ensure that every reasonable effort is made to collect all PK blood samples at the protocol scheduled time.

### Endpoints and Statistical Analysis

### Sample Size Calculation and Power Consideration

Change in hemoglobin at 12 weeks are the primary study endpoint and is used to calculate the study sample size. Group sample sizes of 80 and 80 achieve 94% power to reject the null hypothesis of equal means when the mean difference is 1.0 mmHg with a standard deviation for both groups of 1.8 mmHg. The test statistic is a two-sample t-test at a two-sided significance level (alpha) of 0.05.

### Study Population and Analysis Sets

The “Safety Set” is separately defined for subjects who have received at least 1 dose of BGE-117 or who have received only placebo. The safety population, analyzed as treated is used to report safety.

The “Full Analysis Set/Intent-to-treat population (SAF/ITT)” is defined as subjects who were randomized to the study and received at least one dose of study drug and had one post-baseline assessment. The FAS/ITT analyzed as randomized is used to analyze efficacy endpoints.

The “Per Protocol Set (PPS)” is defined as subjects in the FAS/ITT set with no major protocol deviations. This set may be formed if >5% of subjects in FAS/ITT. Efficacy analyses may be repeated for the PPS set, analyzed as treated.

The “PK” set consists of subjects who have received at least 1 dose of BGE-117 and have at least 1 evaluable post-dose PK concentration value. This set is used to analyze PK.

The “Proteomics, Metabolomic and Transcriptomic” set consists of subjects who have received at least 1 dose of BGE-117 and have at least 1 evaluable post-dose proteomic and transcriptomic value. This set is used to analyze proteomics, metabolomic and transcriptomic.

### Endpoints

### Primary Endpoint:

Improvement in Hb at Visit 15 (Day 85/EOTP) compared to baseline

### Key Secondary Endpoint:

Improvement in FACIT-Fatigue Score at Visit 15 (Day 85/EOTP) compared to baseline

### Secondary Endpoints:

Change in Hb at Visits 7, and 11 (Days 29, and 57) and Follow-up Visit compared to baseline
Improvement in FACIT-Fatigue score at Visits 7 and 11 (Day 29 and 57) and the Follow-up Visit compared to baseline
Change in SPPB Score at Visits 7, 11, and 15 (Day 29, 57, and 85), and Follow-up Visit compared to baseline
Change in 6MWT Distance at Visits 7, 11, and 15 (Days 29, 57, and 85) and the Follow-up Visit compared to baseline
Change in SF-36 at Visits 7, 11, and 15 (Days 29, 57, and 85) and the Follow-up Visit compared to baseline
Evaluate the starting dose of BGE-117 in the treatment of UAA for subjects with eGFR ≥60 mL/min/1.73 m² and subjects with eGFR ≥30 and <60 mL/min/1.73 m²

### Efficacy Statistical Analysis

Change in Hb is the primary endpoint and is a direct assessment of the disease of unexplained anemia in aging patients. Changes in FACIT-Fatigue is the key secondary endpoint. Analysis of both of these endpoints is an ANCOVA with baseline covariates. Testing for FACIT-Fatigue is done only after achieving statistical significance for the change in Hb.

Primary analysis is an analysis of covariance (ANCOVA) with baseline Hb as a covariate. Additional potential covariates are investigated. Details regarding analysis of this primary and all secondary endpoints are given in the SAP.

### Safety Analysis

Safety analyses is performed based on the corresponding Safety Set. Subjects who receive a placebo may be pooled per study part in the safety analyses.

Adverse events are coded using the Medical Dictionary for Regulatory Activities (MedDRA). The number of events, incidence, and percentage of treatment-emergent AEs (TEAEs) are calculated overall by system organ class, preferred term, and treatment group for each cohort and treatment group. The number and percentage of subjects with TEAEs are further summarized by severity and relationship to the IP. Adverse events related to IP, AEs leading to withdrawal, SAEs, and deaths are similarly summarized/listed.

Clinical laboratory tests, vital signs, ophthalmology examinations, vascular Doppler findings, and ECG findings are summarized by treatment group and visit. Descriptive statistics are calculated for quantitative safety data as well as for the difference from baseline, if applicable. Frequency counts are compiled for the classification of qualitative safety data. The baseline for safety data are defined as the last value prior to the first dose of IP. Potentially clinically important findings are summarized or listed.

### Clinical Outcomes Assessments

Clinical outcomes assessments are assessed at various time points.

### Functional Assessment of Chronic Illness Therapy-Fatigue Scale (FACIT-Fatigue)

FACIT-Fatigue assessments are collected from consenting subjects. The FACIT-Fatigue scale is a 13-item questionnaire that assesses self-reported fatigue and its impact on daily activities and function.

### Item Short Form Survey Instrument (SF-36)

SF-36 assessments are collected from consenting subjects. The SF-36 acute version is a general health status questionnaire designed to elucidate the subject's perception of health on several domains, including physical functioning, role physical, bodily pain, vitality, social functioning, role emotional, mental health, and general health over the past 7 days. The questionnaire contains 36 questions that ask the subject to recall how he/she felt during the past 7 days.

### Short Physical Performance Battery (SPPB)

SPPB assessments are collected from consenting subjects. The SPPB consists of 3 parts: a Balance Test, a Gait Speed Test, and a Chair Stand Test.

### Six-Minute Walk Test (6MWT)

6MWT assessments are collected from consenting subjects. The 6MWT consists of the subject walking for a total of 6 minutes on a hard, flat surface.

### Clinical Global Impression (CGI)

CGI assessments are collected from consenting subjects. The CGI is a questionnaire-based instrument for making global assessments of subjects' progress and treatment response over time. This study will collect 2 subscales of the CGI:
Change of Condition
Therapeutic Efficacy

### CGI-Change of Condition

The CGI-Change of Condition measures is to be completed by the subject. The subject is to measure any change in their energy level using a 7-point scale consisting of the following categories:
1—Very much improved
2—Much improved
3—Minimally improved
4—No change
5—Minimally worse
6—Much worse
7—Very much worse

### CGI-Therapeutic Efficacy

The CGI-Therapeutic Efficacy is measured, by the subject, as to the perceived therapeutic efficacy of treatment using a 4-point scale consisting of the following categories:
1—Very good
2—Moderate
3—Slight
4—Unchanged or worse

### Grip Strength Test (Upper Body Strength Test)

The Jamar Hand Dynamometer grip strength test is collected from consenting subjects. This assessment is to record the subject's maximal handgrip strength. Each participant performs 3 maximum voluntary tests for each hand, as instructed.

The test starts with the dominant hand. The test is supervised by an investigator or appropriately delegated employee. Where possible, the subject is assessed by the same person throughout a subject's study participation. The results of each test are measured, in kg, to 1 decimal point.

### Results

Administration of BGE-117 is effective to:
1) Increase hemoglobin levels as compared to baseline
2) Reduce fatigue, as demonstrated by the improvement in FACIT-Fatigue Score, as compared to baseline
3) Improve physical activity as demonstrated by improved short physical performance battery (SPPB) scores, 6-minute walk test (6MWT), and grip strength test, as compared to baseline
4) Improve oxygen delivery, mobility, QOL, and energy levels
5) Improve activity levels and sleep quality as compared to baseline.

### 6. EQUIVALENTS AND INCORPORATION BY REFERENCE

While the disclosure has been particularly shown and described with reference to a preferred embodiment and various alternate embodiments, it will be understood by persons skilled in the relevant art that various changes in form and details can be made therein without departing from the spirit and scope of the disclosure.

All references, issued patents and patent applications cited within the body of the instant specification are hereby incorporated by reference in their entirety, for all purposes.

1. A method of treating an aging-related morbidity, comprising:
administering a therapeutically effective amount of a hypoxia-inducible factor prolyl hydroxylase (HIF-PH) inhibitor to a human subject who is at least 65 years old who has, or is at risk for developing, an aging-related morbidity.

2. The method of claim 1, wherein the aging-related morbidity is anemia of aging.

3. The method of claim 2, wherein the anemia of aging is anemia of inflammation (AI) in the elderly.

4. The method of claim 2, wherein the age-related morbidity is anemia induced by acute medical events, processes, or hospital admissions.

5. The method of claim 3, wherein the human subject has a CRP level of greater than 2 mg/L.

6. (canceled)

7. (canceled)

8. (canceled)

9. (canceled)

10. The method of claim 2, wherein the anemia is an unexplained anemia in the elderly (UAE).

11. The method of claim 10, wherein the human subject has a CRP level of less than 10 mg/L.

12. (canceled)

13. (canceled)

14. (canceled)

15. (canceled)

16. (canceled)

17. The method of claim 1, wherein the human subject has a pre-treatment IL-6 level of greater than 2.5 pg/ml.

18. (canceled)

19. (canceled)

20. The method of claim 1, wherein the human subject has a pre-treatment TNFα level of greater than 7 pg/ml.

21. (canceled)

22. (canceled)

23. (canceled)

24. The method of claim 1, wherein the human subject has a pre-treatment hemoglobin level below 12 g/dL.

25. (canceled)

26. (canceled)

27. (canceled)

28. (canceled)

29. The method of claim 1, wherein the human subject has an eGFR of greater than 30 ml/min.

30. (canceled)

31. (canceled)

32. The method of claim 1, wherein the human subject has serum ferritin (SF) greater than 100 and/or tumor inflammation signature (TIS) greater than 20%.

33. The method of claim 1, wherein the human subject does not have a renal disease or chronic kidney disease (CKD).

34. (canceled)

35. (canceled)

36. The method of claim 1, wherein the human subject is not on hemodialysis.

37. (canceled)

38. The method of claim 1, wherein the age-related morbidity is frailty.

39. The method of claim 1, wherein the human subject has:
reduced muscle strength and/or reduced hand grip strength;
reduced muscle force;
reduction in lower limb muscle mass;
upper limb muscle mass; or
a reduction in muscle volume.

40. (canceled)

41. (canceled)

42. (canceled)

43. (canceled)

44. (canceled)

45. The method of claim 1, wherein the human subject has not been diagnosed with any disease except age-related frailty.

46. The method of claim 39, wherein the human subject has sarcopenia.

47. The method of claim 1, wherein the human subject has a reduction in capillary density.

48. The method of claim 1, wherein the age-related morbidity is fatigue.

49. (canceled)

50. (canceled)

51. (canceled)

52. (canceled)

53. (canceled)

54. (canceled)

55. (canceled)

56. (canceled)

57. The method of claim 1, wherein the HIF-PH inhibitor is a compound represented by the following general formula (I′): 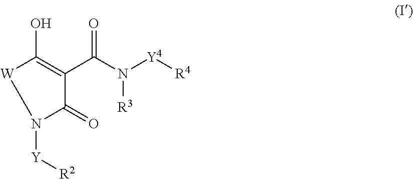
wherein in formula (I′), W represents the formula —CR¹¹R¹²CR¹³R¹⁴;
R¹¹ represents a hydrogen atom, C₁₋₄ alkyl, or phenyl;
R¹² represents a hydrogen atom, a fluoride atom or C₁₋₄ alkyl; or
R¹¹ and R¹², together with the adjacent carbon atom, form a C₃₋₈ cycloalkane or a 4- to 8-membered saturated heterocycle containing an oxygen atom;
R¹³ represents a hydrogen atom, carbamoyl, C₁₋₄ alkyl, wherein the C₁₋₄ alkyl is optionally substituted by one group selected from the group consisting of hydroxy, C₁₋₃ alkoxy, and di-C₁₋₃ alkylamino, halo-C₁₋₄ alkyl, phenyl, pyridyl, benzyl, or phenethyl;
R¹⁴ represents a hydrogen atom, C₁₋₄ alkyl, or halo-C₁₋₄ alkyl; or
R¹³ and R¹⁴, together with the adjacent carbon atom, form a C₃₋₈ cycloalkane, a 4- to 8-membered saturated heterocycle containing an oxygen atom, or a 4- to 8-membered saturated heterocycle containing a nitrogen atom, wherein the 4- to 8-membered saturated heterocycle containing a nitrogen atom is optionally substituted by one or two groups which are the same or different and are selected from the group consisting of methyl, benzyl, phenylcarbonyl, and oxo; or
R¹² and R¹³, together with the adjacent carbon atoms, form a C₃₋₈ cycloalkane;
Y represents a single bond or C₁₋₆ alkanediyl, wherein the C₁₋₆ alkanediyl is optonally substituted by one hydroxyl, and one of the carbon atoms in the C₁₋₆ alkanediyl is optionally substituted by C₃₋₆ cycloalkane1,1-diyl;
R² represents:
a hydrogen atom,
C₁₋₆ akyl,
C₃₋₈ cycloalkyl, wherein the C₃₋₈ cycloalkyl is optionally substituted by one or two groups which are the same or different and are selected from the group consisting of C₁₋₆ alkyl which is optionally substituted by one phenyl, phenyl which is optionally substituted by one group selected from the group consisting of a halogen atom and halo-C₁₋₆ alkyl, C₁₋₆ alkoxy which is optionally substituted by one group selected from the group consisting of C₃₋₈ cycloalkyl, phenyl optionally substituted by one group selected from the group consisting of a halogen atom and C₁₋₆ alkyl, and pyridyl optionally substituted by one halogen atom, C₃₋₈ cycloalkoxy, phenoxy which is optionally substituted by one group selected from the group consisting of a halogen atom, C₁₋₆ alkyl, C₃₋₈ cycloalkyl, and halo-C₁₋₆ alkyl, and pyridyloxy which is optionally substituted by one group selected from the group consisting of a halogen atom, C₁₋₆ alkyl, C₃₋₈ cycloalkyl, and halo-C₁₋₆ alkyl,
phenyl, wherein the phenyl is optionally substituted by one to three groups which are the same or different and are selected from group α3 of substituents,
naphthyl,
indanyl,
tetrahydronaphthyl,
pyrazolyl,
imidazolyl,
isoxazolyl,
oxazolyl, wherein the pyrazolyl, imidazolyl, isoxazolyl, and oxazolyl are optionally substituted by one or two groups which are the same or different and are selected from the group consisting of C₁₋₆ alkyl and phenyl which is optionally substituted by one group selected from the group consisting of a halogen atom and C₁₋₆ alkyl,
thiazoyl, wherein the thiazoyl is optionally substituted by one or two groups which are the same or different and are selected from the group consisting of C₁₋₆ alkyl, phenyl which is optionally substituted by one group selected from the group consisting of a halogen atom and C₁₋₆ alkyl, and morpholino,
pyridyl, wherein the pyridyl is optionally substituted by one or two groups which are the same or different and are selected from group α5 of substituents,
pyridazinyl,
pyrimidinyl,
pyrazinyl,
wherein the pyridazinyl, pyrimidinyl, and pyrazinyl are optionally substituted by one group selected from the group consisting of C₁₋₆ alkyl, halo-C₁₋₆ alkyl, C₃₋₈ cycloalkyl, phenyl, C₁₋₆ alkoxy which is optionally substituted by one C₃₋₈ cycloalkyl, and phenoxy which is optionally substituted by one group selected from the group consisting of a halogen atom, C₁₋₆ alkyl, and C₃₋₈ cycloalkyl,
benzothiophenyl,
quinolyl,
methylenedioxyphenyl, wherein the methylenedioxyphenyl is optionally substituted by one or two fluorine atoms,
4- to 8-membered saturated heterocyclyl containing a nitrogen atom, wherein the 4- to 8-membered saturated heterocyclyl containing a nitrogen atom is optionally substituted by one group selected from the group consisting of pyrimidinyl, phenyl-C₁₋₃ alkyl, C₃₋₈ cycloalkyl-C₁₋₃ alkylcarbonyl, and phenyl-C₁₋₃ alkoxycarbonyl, or
the following formula (I″)
—CONR⁵CH₂—R⁶ (I″), wherein in formula (I″):
R⁵ represents a hydrogen atom or C₁₋₃ alkyl, and R⁶ represents phenyl which is optionally substituted by one group selected from the group consisting of a halogen atom, C₁₋₆ alkyl, halo-C₁₋₆ alkyl, and phenyl,
group α3 of substituents consists of:
hydroxy,
cyano,
carboxy,
a halogen atom,
C₁₋₆ alkyl, wherein the C₁₋₆ alkyl is optionally substituted by one group selected from the group consisting of C₃₋₈ cycloalkyl, phenyl, C₁₋₆ alkoxy which is optionally substituted by one C₃₋₈ cycloalkyl optionally substituted by one C₁₋₆ alkyl , phenoxy which is optionally substituted by one C₁₋₆ alkyl, and pyridyloxy which is optionally substituted by one group selected from the group consisting of C₁₋₆ alkyl and halo-C₁₋₆ alkyl,
halo-C₁₋₆ alkyl,
C₃₋₈ cycloalkyl, wherein the C₃₋₈ cycloalkyl is optionally substituted by one or two halogen atoms,
C₃₋₈ cycloalkenyl, wherein the C₃₋₈ cycloalkenyl is optionally substituted by one or two halogen atoms,
phenyl, wherein the phenyl is optionally substituted by one to three groups which are the same or different and are selected from group α4 of substituents,
thienyl, wherein the thienyl is optionally substituted by one C₁₋₆ alkyl,
pyrazolyl, wherein the pyrazolyl is optionally substituted by one C₁₋₆ alkyl,
isoxazolyl,
thiazoyl, wherein the thiazoyl is optionally substituted by one or two groups which are the same or different and are selected from the group consisting of hydroxy, C₁₋₆ alkyl, and C₁₋₆ alkoxy,
pyridyl, wherein the pyridyl is optionally substituted by one group selected from the group consisting of carboxy, hydroxy, amino, a halogen atom, C₁₋₆ alkyl, halo-C₁₋₆ alkyl, C₃₋₈ cycloalkyl, C₁₋₆ alkoxy, halo-C₁₋₆ alkoxy, and C₁₋₆ alkylsulfonyl,
pyrimidinyl, wherein the pyrimidinyl is optionally substituted by one amino,
quinolyl,
C₁₋₆ alkoxy, wherein the C₁₋₆ alkoxy is optionally substituted by one group selected from the group consisting of carboxy, hydroxy, carbamoyl, C₃₋₈ cycloalkyl which is optionally substituted by one C₁₋₆ alkyl, phenyl which is optionally substituted by one group selected from the group consisting of hydroxy, a halogen atom, C₁₋₆ alkyl, halo-C₁₋₆ alkyl, C₁₋₆ alkoxy, halo-C₁₋₆ alkoxy, and di-C₁₋₆ alkylamino, pyridyl which is optionally substituted by one group selected from the group consisting of a halogen atom and C₁₋₆ alkyl, benzotriazolyl, imidazothiazoyl, di-C₁₋₆ alkylamino, oxazolyl which is optionally substituted by one or two C₁₋₆ alkyls, pyrazolyl, which is optionally substituted by one or two C₁₋₆ alkyls, thiazoyl which is optionally substituted by one C₁₋₆ alkyl, and indazolyl which is optionally substituted by one C₁₋₆ alkyl,
halo-C₁₋₆ alkoxy,
C₂₋₆ alkenyloxy,
C₃₋₈ cycloalkoxy,
phenoxy, wherein the phenoxy is optionally substituted by one or two groups which are the same or different and are selected from the group consisting of a halogen atom, C₁₋₆ alkyl, halo-C₁₋₆ alkyl, C₁₋₆ alkoxy, and halo-C₁₋₆ alkoxy,
pyridyloxy, wherein the pyridyloxy is optionally substituted by one group selected from the group consisting of a halogen atom, C₁₋₆ alkyl, halo-C₁₋₆ alkyl, and C₃₋₈ cycloalkyl,
pyrimidinyloxy,
piperazinyl, wherein the piperazinyl is optionally substituted by one C₁₋₆ alkyl,
mono-C₁₋₆ alkylaminocarbonyl, wherein the C₁₋₆ alkyl in the mono-C₁₋₆ alkylaminocarbonyl is optionally substituted by one group selected from the group consisting of carboxy, hydroxy, di-C₁₋₆ alkylamino, pyridyl, phenyl, and 2-oxopyrrolidinyl,
di-C₁₋₆ alkylaminocarbonyl, wherein the two C₁₋₆ alkyls in the di-C₁₋₆ alkylaminocarbonyl, together with the adjacent nitrogen atom, optionally form a 4- to 8-membered saturated heterocycle containing a nitrogen atom,
C₁₋₆ alkylsulfanyl, and
C₁₋₆ alkylsulfonyl;
group α4 of substituents consists of:
carboxy,
cyano,
hydroxy,
sulfamoyl,
a halogen atom,
C₁₋₆ alkyl,
halo-C₁₋₆ alkyl,
C₃₋₈ cycloalkyl,
phenyl,
C_{1.6} alkoxy,
halo-C₁₋₆ alkoxy,
C₁₋₆ alkylcarbonyl,
di-C₁₋₆ alkylaminocarbonyl,
C₁₋₆ alkylsulfonyl,
mono-C₁₋₆ alkylaminosulfonyl, wherein the C₁₋₆ alkyl in the mono-C₁₋₆ alkylaminosulfonyl is optionally substituted by one hydroxy, and di-C₁₋₆ alkylaminosulfonyl;
group α5 of substituents consist of:
a halogen atom,
C₁₋₆ alkyl,
halo-C₁₋₆ alkyl,
C₁₋₆ alkoxy, wherein the C₁₋₆ alkoxy is optionally substituted by one group selected from the group consisting of C₃₋₈ cycloalkyl which is optionally substituted by one C₁₋₆ alkyl and phenyl which is optionally substituted by one group selected from the group consisting of a halogen atom and C₁₋₆ alkyl,
halo-C₁₋₆ alkoxy,
phenyl, wherein the phenyl is optionally substituted by one group selected from group α6 of substituents,
pyridyl,
phenoxy, wherein the phenoxy is optionally substituted by one or two groups which are the same or different and are selected from the group consisting of a halogen atom, cyano, C₁₋₆ alkyl, halo-C₁₋₆ alkyl, C₃₋₈ cycloalkyl, halo-C₁₋₆ alkoxy and C₁₋₆ alkoxy which is optionally substituted by one phenyl, and,
pyridyloxy, wherein the pyridyloxy is optionally substituted by one C₁₋₆ alkyl, and
phenylsulfanyl, wherein the phenylsulfanyl is optionally substituted by one halogen atom;
group α6 of substituents consists of:
a halogen atom,
C₁₋₆ alkyl,
halo-C₁₋₆ alkyl,
C₃₋₈ cycloalkyl,
C₁₋₆ alkoxy, and
halo-C₁₋₆ alkoxy;
Y⁴ represents C₁₋₄ alkanediyl;
R³ represents a hydrogen atom or methyl;
R⁴ represents —COOH, —CONHOH, or tetrazolyl;
or a pharmaceutically acceptable salt thereof.

58. The method of claim 57, wherein in the aforementioned general formula (I′):
Y⁴ is methanediyl,
R³ is a hydrogen atom,
R⁴ is —COOH,
or a pharmaceutically acceptable salt thereof.

59. The method of claim 58, wherein in the aforementioned general formula (I′), the compound is represented by general formula (I′-2): 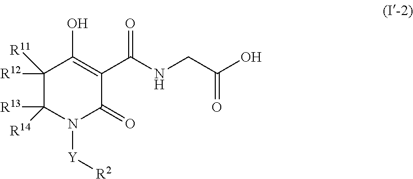
wherein in formula (I′-2):
R¹¹ is a hydrogen atom, a fluorine atom, C₁₋₄ alkyl, or phenyl,
R¹² is a hydrogen atom, a fluorine atom, or C₁₋₄ alkyl, or
R¹¹ and R¹², together with the adjacent carbon atom, form a C₃₋₈ cycloalkane or a 4- to 8-membered saturated heterocycle containing an oxygen atom;
R¹³ is a hydrogen atom, carbamoyl, C₁₋₄ alkyl, wherein the C₁₋₄ alkyl is optionally substituted by one group selected from the group consisting of hydroxy, C₁₋₃ alkoxy, and di-C₁₋₃ alkylamino, halo-C₁₋₄ alkyl, phenyl, pyridyl, benzyl, or phenethyl;
R¹⁴ is a hydrogen atom, C₁₋₄ alkyl, or halo-C₁₋₄ alkyl, or
R¹³ and R¹⁴, together with the adjacent carbon atom, form a C₃₋₈ cycloalkane, a 4- to 8-membered saturated heterocycle containing an oxygen atom, or a 4- to 8-membered saturated heterocycle containing a nitrogen atom, wherein the 4- to 8-membered saturated heterocycle containing a nitrogen atom is optionally substituted by one or two groups which are the same or different and are selected from the group consisting of methyl, benzyl, phenylcarbonyl, and oxo, or
R¹² and R¹³, together with the adjacent carbon atoms, form a C₃₋₈ cycloalkane,
or a pharmaceutically acceptable salt thereof.

60. The method of claim 59, wherein in the aforementioned general formula (I′-2):
Y is a single bond or C₁₋₆ alkanediyl, wherein one of the carbon atoms in the C₁₋₆ alkanediyl is optionally substituted by C₃₋₆ cycloalkane-1,1-diyl,
R² is:
C₃₋₈ cycloalkyl, wherein the C₃₋₈ cycloalkyl is optionally substituted by one or two groups which are the same or different and are selected from the group consisting of C₁₋₆ alkyl which is optionally substituted by one phenyl, phenyl which is optionally substituted by one halo-C₁₋₆ alkyl, C₁₋₆ alkoxy which is optionally substituted by one group selected from the group consisting of C₃₋₈ cycloalkyl, phenyl optionally substituted by one group selected from the group consisting of a halogen atom and C₁₋₆ alkyl, and pyridyl optionally substituted by one halogen atom, C₃₋₈ cycloalkoxy, phenoxy which is optionally substituted by one group selected from the group consisting of a halogen atom, C₁₋₆ alkyl, C₃₋₈ cycloalkyl, and halo-C₁₋₆ alkyl, and pyridyloxy which is optionally substituted by one group selected from the group consisting of a halogen atom, C₁₋₆ alkyl, C₃₋₈ cycloalkyl, and halo-C₁₋₆ alkyl,
phenyl, wherein the phenyl is optionally substituted by one to three groups which are the same or different and are selected from the aforementioned group α3 of substituents,
naphthyl,
indanyl,
tetrahydronaphthyl,
pyrazolyl, wherein the pyrazolyl is optionally substituted by one or two groups which are the same or different and are selected from the group consisting of C₁₋₆ alkyl and phenyl which is optionally substituted by one C₁₋₆ alkyl,
imidazolyl, wherein the imidazolyl is optionally substituted by one group selected from the group consisting of C₁₋₆ alkyl and phenyl,
isoxazolyl, wherein the isoxazolyl is optionally substituted by one phenyl which is optionally substituted by one halogen atom,
oxazolyl, wherein the oxazolyl is optionally substituted by one or two groups which are the same or different and are selected from the group consisting of C₁₋₆ alkyl and phenyl,
thiazoyl, wherein the thiazoyl is optionally substituted by one group selected from the group consisting of C₁₋₆ alkyl, phenyl, and morpholino,
pyridyl, wherein the pyridyl is optionally substituted by one or two groups which are the same or different and are selected from the aforementioned group α5 of substituents,
pyridazinyl, wherein the pyridazinyl is optionally substituted by one C₁₋₆ alkoxy which is optionally substituted by one C₃₋₈ cycloalkyl,
pyrimidinyl, wherein the pyrimidinyl is optionally substituted by one group selected from the group consisting of halo-C₁₋₆ alkyl, C₃₋₈ cycloalkyl, phenyl, and phenoxy which is optionally substituted by one C₁₋₆ alkyl,
pyrazinyl, wherein the pyrazinyl is optionally substituted by one group selected from the group consisting of C₁₋₆ alkoxy which is optionally substituted by one C₃₋₈ cycloalkyl and phenoxy which is optionally substituted by one group selected from the group consisting of a halogen atom, C₁₋₆ alkyl, and C₃₋₈ cycloalkyl,
benzothiophenyl,
quinolyl, or
methylenedioxyphenyl, wherein the methylenedioxyphenyl is optionally substituted by one or two fluorine atoms,
or a pharmaceutically acceptable salt thereof.

61. The method of claim 60, wherein in the aforementioned general formula (I′-2):
R¹¹ is a hydrogen atom,
R¹² is a hydrogen atom,
R¹³ is a hydrogen atom,
R¹⁴ is a hydrogen atom,
Y is methanediyl,
R² is:
phenyl, wherein the phenyl is substituted by one group selected from the group consisting of phenyl which is optionally substituted by one or two groups which are the same or different and are selected from the group consisting of carboxy, cyano, hydroxy, sulfamoyl, a halogen atom, C₁₋₆ alkyl, halo-C₁₋₆ alkyl, C₃₋₈ cycloalkyl, phenyl, C₁₋₆ alkoxy, halo-C₁₋₆ alkoxy, C₁₋₆ alkylcarbonyl, di-C₁₋₆ alkylaminocarbonyl, C₁₋₆ alkyl sulfonyl, di-C₁₋₆ alkylaminosulfonyl and mono-C₁₋₆ alkylaminosulfonyl, wherein the C₁₋₆ alkyl in the mono-C₁₋₆alkylaminosulfonyl is optionally substituted by one hydroxy, pyridyl which is optionally substituted by one group selected from the group consisting of carboxy, hydroxy, amino, a halogen atom, C₁₋₆ alkyl, halo-C₁₋₆ alkyl, C₃₋₈ cycloalkyl, C₁₋₆ alkoxy, and C₁₋₆ alkylsulfonyl, phenoxy which is optionally substituted by one or two groups which are the same or different and are selected from the group consisting of a halogen atom, C₁₋₆ alkyl, C₁₋₆ alkoxy, and halo-C₁₋₆ alkoxy, and pyridyloxy which is optionally substituted by one group selected from the group consisting of a halogen atom, C₁₋₆ alkyl, halo-C₁₋₆ alkyl, and C₃₋₈ cycloalkyl, and said substituted phenyl represented by R² may further be substituted by one halogen atom;
pyridyl, wherein the pyridyl is substituted by one group selected from the group consisting of phenyl which is optionally substituted by one group selected from the group consisting of a halogen atom, C₁₋₆ alkyl, halo-C₁₋₆ alkyl, C₃₋₈ cycloalkyl, C₁₋₆ alkoxy, and halo-C₁₋₆ alkoxy, pyridyl, phenoxy which is optionally substituted by one or two groups which are the same or different and are selected from the group consisting of a halogen atom, cyano, C₁₋₆ alkyl, halo-C₁₋₆ alkyl, C₃₋₈ cycloalkyl, halo-C₁₋₆ alkoxy and C₁₋₆ alkoxy optionally substituted by one phenyl, and pyridyloxy which is optionally substituted by one C₁₋₆ alkyl, and said substituted pyridyl represented by R² may further be substituted by one group selected from the group consisting of a halogen atom and C₁₋₆ alkyl; or
pyrazinyl which is substituted by one phenoxy wherein the phenoxy is optionally substituted by one group selected from the group consisting of a halogen atom, C₁₋₆ alkyl, and C₃₋₈ cycloalkyl,
or a pharmaceutically acceptable salt thereof.

62. The method of claim 57, wherein the compound is selected from:
N-{[4-hydroxy-2-oxo-1-(4-phenoxybenzyl)-1,2,5,6-tetrahydro-3-pyridinyl]carbonyl}glycine;
N-[(4-hydroxy-1-{1[6-(4-methylphenoxy)-3-pyridinyl]methyl}-2-oxo-1,2,5,6-tetrahydro-3-pyridinyl)carbonyl]glycine;
N-({4-hydroxy-2-oxo-1-[(6-phenoxy-3-pyridinyl)methyl]-1,2,5,6-tetrahydro-3-pyridinyl}carbonyl)glycine;
N-({1-[4-(4-fluorophenoxy)benzyl]-4-hydroxy-2-oxo-1,2,5,6-tetrahydro-3-pyridinyl}carbonyl)glycine;
N-({4-hydroxy-1-[4-(4-methylphenoxy)benzyl]-2-oxo-1,2,5,6-tetrahydro-3-pyridinyl}carbonyl)glycine;
N-[(1-{[6-(4-cyanophenoxy)-3-pyridinyl]methyl}-4-hydroxy-2-oxo-1,2,5,6-tetrahydro-3-pyridinyl)carbonyl]glycine;
N-({4-hydroxy-2-oxo-1-[4-(2-pyrimidinyloxy)benzyl]-1,2,5,6-tetrahydro-3-pyridinyl}carbonyl)glycine;
N-[(1-{[6-(4-fluorophenoxy)-3-pyridinyl]methyl}-4-hydroxy-2-oxo-1,2,5,6-tetrahydro-3-pyridinyl)carbonyl]glycine;
N-[(1{[-6-(4-chlorophenoxy)-3-pyridinyl]methyl}-4-hydroxy-2-oxo-1,2,5,6-tetrahydro-3-pyridinyl)carbonyl]glycine;
N-{[4-hydroxy-2-oxo-1-({6-[4-(trifluoromethyl)phenoxy]-3-pyridinyl}methyl)-1,2,5,6-tetrahydro-3-pyridinyl]carbonyl}glycine;
N-[(4-hydroxy-1-{[6-(3-methylphenoxy)-3-pyridinyl]methyl}-2-oxo-1,2,5,6-tetrahydro-3-pyridinyl)carbonyl]glycine;
N-[(1-{[6-(3-fluorophenoxy)-3-pyridinyl]methyl}-4-hydroxy-2-oxo-1,2,5,6-tetrahydro-3-pyridinyl)carbonyl]glycine;
N-({4-hydroxy-1-[4-(3-methylphenoxy)benzyl]-2-oxo-1,2,5,6-tetrahydro-3-pyridinyl}carbonyl)glycine;
N-({1-[4-(3-fluorophenoxy)benzyl]-4-hydroxy-2-oxo-1,2,5,6-tetrahydro-3-pyridinyl}carbonyl)glycine;
N-[1-{[5-(4-fluorophenoxy)-2-pyridinyl]methyl}-4-hydroxy-2-oxo-1,2,5,6-tetrahydro-3-pyridinyl)carbonyl]glycine;
N-[(4-hydroxy-1-{[5-(4-methylphenoxy)-2-pyridinyl]methyl}-2-oxo-1,2,5,6-tetrahydro-3-pyridinyl)carbonyl]glycine;
N-({1-[4-(4-chlorophenoxy)benzyl]-4-hydroxy-2-oxo-1,2,5,6-tetrahydro-3-pyridinyl}carbonyl)glycine;
N-[(4-hydroxy-1-{4-[(6-methyl-3-pyridinyl)oxy]benzyl}-2-oxo-1,2,5,6-tetrahydro-3-pyridinyl)carbonyl]glycine;
N-[(1-{[6-(2-fluorophenoxy)-3-pyridinyl]methyl}-4-hydroxy-2-oxo-1,2,5,6-tetrahydro-3-pyridinyl)carbonyl]glycine;
N-[(4-hydroxy-1-{[6-(2-methylphenoxy)-3-pyridinyl]methyl}-2-oxo-1,2,5,6-tetrahydro-3-pyridinyl)carbonyl]glycine;
N-({1-[4-(2-fluorophenoxy)benzyl]-4-hydroxy-2-oxo-1,2,5,6-tetrahydro-3-pyridinyl}carbonyl)glycine;
N-({4-hydroxy-1-[4-(2-methylphenoxy)benzyl]-2-oxo-1,2,5,6-tetrahydro-3-pyridinyl}carbonyl)glycine;
N-[(1-{[6-(3-chlorophenoxy)-3-pyridinyl]methyl}-4-hydroxy-2-oxo-1,2,5,6-tetrahydro-3-pyridinyl)carbonyl]glycine;
N-{[4-hydroxy-2-oxo-1-({6-[3-(trifluoromethyl)phenoxy]-3-pyridinyl}methyl)-1,2,5,6-tetrahydro-3-pyridinyl]carbonyl}glycine;
N-({4-hydroxy-1-[4-(3-methoxyphenoxy)benzyl]-2-oxo-1,2,5,6-tetrahydro-3-pyridinyl}carbonyl)glycine;
N-{[4-hydroxy-2-oxo-1-({6-[3-(trifluoromethoxy)phenoxy]-3-pyridinyl}methyl)-1,2,5,6-tetrahydro-3-pyridinyl]carbonyl}glycine;
N-[(1-{4-[(5-fluoro-2-pyridinyl)oxy]benzyl}-4-hydroxy-2-oxo-1,2,5,6-tetrahydro-3-pyridinyl)carbonyl]glycine;
N-[(1-{4-[(5-chloro-2-pyridinyl)oxy]benzyl}-4-hydroxy-2-oxo-1,2,5,6-tetrahydro-3-pyridinyl)carbonyl]glycine;
N-[1-{[(6-(4-cyclopropylphenoxy)-3-pyridinyl]methyl}-4-hydroxy-2-oxo-1,2,5,6-tetrahydro-3-pyridinyl)carbonyl]glycine;
N-[(4-hydroxy-1-{4-[(5-methyl-2-pyridinyl)oxy]benzyl}-2-oxo-1,2,5,6-tetrahydro-3-pyridinyl)carbonyl]glycine;
N-{[4-hydroxy-2-oxo-1-(4-{[5-(trifluoromethyl)-2-pyridinyl]oxy}benzyl)-1,2,5,6-tetrahydro-3-pyridinyl]carbonyl}glycine;
N-{[4-hydroxy-1-({5-methyl-6-[(6-methyl-3-pyridinyl)oxy]-3-pyridinyl}methyl)-2-oxo-1,2,5,6-tetrahydro-3-pyridinyl]carbonyl}glycine;
N-[(1-{[5-(4-chlorophenoxy)-2-pyridinyl]methyl}-4-hydroxy-2-oxo-1,2,5,6-tetrahydro-3-pyridinyl)carbonyl]glycine;
N-[(4-hydroxy-1-{[6-(3-methoxyphenoxy)-3-pyridinyl]methyl}-2-oxo-1,2,5,6-tetrahydro-3-pyridinyl)carbonyl]glycine;
N-[(1-{4-[(6-chloro-3-pyridinyl)oxy]benzyl}-4-hydroxy-2-oxo-1,2,5,6-tetrahydro-3-pyridinyl)carbonyl]glycine;
N-{[4-hydroxy-2-oxo-1-({5-[4-(trifluoromethyl)phenoxy]-2-pyridinyl}methyl)-1,2,5,6-tetrahydro-3-pyridinyl]carbonyl}glycine;
N-{[4-hydroxy-2-oxo-1-(4-{[6-(trifluoromethyl)-3-pyridinyl]oxy}benzyl)-1,2,5,6-tetrahydro-3-pyridinyl]carbonyl}glycine;
N-[(1-{[6-(3-chloro-4-methylphenoxy)-3-pyridinyl]methyl}-4-hydroxy-2-oxo-1,2,5,6-tetrahydro-3-pyridinyl)carbonyl]glycine;
N-[(1-{[6-(3-fluoro-4-methylphenoxy)-3-pyridinyl]methyl}-4-hydroxy-2-oxo-1,2,5,6-tetrahydro-3-pyridinyl)carbonyl]glycine;
N-[(1-{[6-(4-fluoro-3-methylphenoxy)-3-pyridinyl]methyl}-4-hydroxy-2-oxo-1,2,5,6-tetrahydro-3-pyridinyl)carbonyl]glycine;
N-[(1-{[6-(4-ethylphenoxy)-3-pyridinyl]methyl}-4-hydroxy-2-oxo-1,2,5,6-tetrahydro-3-pyridinyl)carbonyl]glycine;
N-[(4-hydroxy-2-oxo-1-{[6-(4-propylphenoxy)-3-pyridinyl]methyl}-1,2,5,6-tetrahydro-3pyridinyl)carbonyl]glycine;
N-[(4-hydroxy-1-{[6-(4-isopropylphenoxy)-3-pyridinyl]methyl}-2-oxo-1,2,5,6-tetrahydro-3-pyridinyl)carbonyl]glycine;
N-[(4-hydroxy-1-{[5-(4-methylphenoxy)-2-pyrazinyl]methyl}-2-oxo-1,2,5,6-tetrahydro-3-pyridinyl)carbonyl]glycine;
N-({1-[4-(3,4-dimethylphenoxy)benzyl]-4-hydroxy-2-oxo-1,2,5,6-tetrahydro-3-pyridinyl}carbonyl)glycine;
N-[(1-{[5-chloro-6-(4-methylphenoxy)-3-pyridinyl]methyl}-4-hydroxy-2-oxo-1,2,5,6-tetrahydro-3-pyridinyl)carbonyl]glycine;
N-[(1-{[5-fluoro-6-(4-methylphenoxy)-3-pyridinyl]methyl}-4-hydroxy-2-oxo-1,2,5,6-tetrahydro-3-pyridinyl)carbonyl]glycine;
N-[(1-{4-[(5-cyclopropyl-2-pyridinyl)oxy]benzyl}-4-hydroxy-2-oxo-1,2,5,6-tetrahydro-3-pyridinyl)carbonyl]glycine;
N-[(4-hydroxy-1-{[2-(4-methylphenoxy)-5-pyrimidinyl]methyl}-2-oxo-1,2,5,6-tetrahydro-3-pyridinyl)carbonyl]glycine; N-[(1-{[6-(4-chlorophenoxy)-5-methyl-3-pyridinyl]methyl}-4-hydroxy-2-oxo-1,2,5,6-tetrahydro-3-pyridinyl)carbonyl]glycine;
N-[(1-{[5-(4-chlorophenoxy)-2-pyrazinyl]methyl}-4-hydroxy-2-oxo-1,2,5,6-tetrahydro-3-pyridinyl)carbonyl]glycine; and
N-[(1-{[5-(4-cyclopropylphenoxy)-2-pyrazinyl]methyl}-4-hydroxy-2-oxo-1,2,5,6-tetrahydro-3-pyridinyl)carbonyl]glycine, or
a pharmaceutically acceptable salt thereof.

63. The method of claim 57, wherein the compound of formula (I′) is represented by general formula (I): 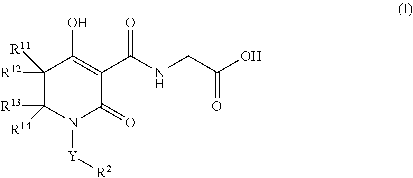
wherein in formula (I):
R¹¹ is a hydrogen atom, C₁₋₄ alkyl, or phenyl,
R¹² is a hydrogen atom or C₁₋₄ alkyl, or
R¹¹ and R¹², together with the adjacent carbon atom, form a C₃₋₈ cycloalkane or a 4- to 8-membered saturated heterocycle containing an oxygen atom;
R¹³ is a hydrogen atom, C₁₋₄ alkyl, halo-C₁₋₄ alkyl, phenyl, benzyl, or phenethyl,
R¹⁴ is a hydrogen atom or C₁₋₄ alkyl, or
R¹³and R¹⁴, together with the adjacent carbon atom, form a C₃₋₈ cycloalkane or a 4- to 8-membered saturated heterocycle containing an oxygen atom, or
R¹² and R¹³, together with the adjacent carbon atoms, form a C₃₋₈ cycloalkane;
Y is a single bond or C₁₋₆ alkanediyl, wherein one of the carbon atoms in the C₁₋₆ alkanediyl is optionally substituted by C₃₋₆ cycloalkane-1,1-diyl;
R² is:
C₃₋₈ cycloalkyl, wherein the C₃₋₈ cycloalkyl is optionally substituted by one group selected from the group consisting of phenyl and benzyl,
phenyl, wherein the phenyl is optionally substituted by one to three groups which are the same or different and are selected from group α1 of substituents,
naphthyl,
indanyl,
tetrahydronaphthyl,
pyrazolyl, wherein the pyrazolyl is substituted by one phenyl, which is optionally substituted by one C₁₋₆ alkyl and may further be substituted by one C₁₋₆ alkyl,
imidazolyl, wherein the imidazolyl is substituted by one phenyl,
isoxazolyl, wherein the isoxazolyl is substituted by one phenyl which is optionally substituted by one halogen atom,
oxazolyl, wherein the oxazolyl is substituted by one phenyl and may further be substituted by one C₁₋₆ alkyl,
thiazoyl, wherein the thiazoyl is substituted by one phenyl,
pyridyl, wherein the pyridyl is substituted by one group selected from the group consisting of phenyl, phenoxy which is optionally substituted by one group selected from the group consisting of a halogen atom, cyano, C₁₋₆ alkyl, halo-C₁₋₆ alkyl, C₃₋₈ cycloalkyl, C₁₋₆ alkoxy, and halo-C₁₋₆ alkoxy), and phenylsulfanyl which is optionally substituted by one halogen atom,
pyrimidinyl, wherein the pyrimidinyl is substituted by one group selected from the group consisting of cyclohexyl and phenyl,
benzothiophenyl,
quinolyl, or
methylenedioxyphenyl, wherein the methylenedioxyphenyl is optionally
substituted by one or two fluorine atoms;
group α1 of substituents consists of:
a halogen atom,
C₁₋₆ alkyl, wherein the C₁₋₆ alkyl is optionally substituted by one group selected from the group consisting of C₃₋₈ cycloalkyl, phenyl, and C₁₋₆ alkoxy which is optionally substituted by one C₃₋₈ cycloalkyl optionally substituted by one C₁₋₆ alkyl,
halo-C₁₋₆ alkyl,
C₃₋₈ cycloalkyl,
phenyl, wherein the phenyl is optionally substituted by one to three groups which are the same or different and are selected from group α2 of substituents,
thienyl,
pyrazolyl, wherein the pyrazolyl is optionally substituted by one C₁₋₆ alkyl,
isoxazolyl,
thiazoyl, wherein the thiazoyl is optionally substituted by one or two C₁₋₆ alkyls,
pyridyl, wherein the pyridyl is optionally substituted by one group selected from the group consisting of C₁₋₆ alkyl, halo-C₁₋₆ alkyl, C₁₋₆ alkoxy, and halo-C₁₋₆ alkoxy,
quinolyl,
C₁₋₆ alkoxy, wherein the C₁₋₆ alkoxy is optionally substituted by one group selected from the group consisting of C₃₋₈ cycloalkyl and phenyl which is optionally substituted by one group selected from the group consisting of a halogen atom and C₁₋₆ alkyl,
halo-C₁₋₆ alkoxy,
C₂₋₆ alkenyloxy,
C₃₋₈ cycloalkoxy,
phenoxy, wherein the phenoxy is optionally substituted by one group selected from the group consisting of a halogen atom, C₁₋₆ alkyl, halo-C₁₋₆ alkyl, C₁₋₆ alkoxy, and halo-C₁₋₆ alkoxy,
pyridyloxy, wherein the pyridyloxy is optionally substituted by one group selected from the group consisting of a halogen atom, C₁₋₆ alkyl, and halo-C₁₋₆ alkyl, and
C₁₋₆alkylsulfanyl;
group α2 of substituents consists of a halogen atom, cyano, hydroxy, C₁₋₆ alkyl, halo-C₁₋₆ alkyl, phenyl, C₁₋₆ alkoxy, halo-C₁₋₆ alkoxy, C₁₋₆ alkylcarbonyl, and di-C₁₋₆ alkylaminosulfonyl,
or a pharmaceutically acceptable salt thereof.

64. The method of claim 62, wherein the compound is N-[(1{[6-(4-chlorophenoxy)-3-pyridinyl]methyl}-4-hydroxy-2-oxo-1,2,5,6-tetrahydro-3-pyridinyl)carbonyl]glycine, or a pharmaceutically acceptable salt thereof.

65. (canceled)

66. (canceled)

67. (canceled)

68. (canceled)

69. (canceled)

70. The method of claim 1, wherein the HIF-PH inhibitor is:
Desidustat, or a pharmaceutically acceptable salt thereof;
Enarodustat, or a pharmaceutically acceptable salt thereof;
Molidustat, or a pharmaceutically acceptable salt thereof;
Roxadustat, or a pharmaceutically acceptable salt thereof;
Daprodustat, or a pharmaceutically acceptable salt thereof;
Vadadustat, or a pharmaceutically acceptable salt thereof;
1-(6-(2,6-dimethylphenoxy)-7-fluoro-4-oxo-3,4-dihydroquinazolin-2-yl)-1H-pyrazole-4-carboxylic acid (JNJ-42905343); or
JNJ-42041935.

71. (canceled)

72. (canceled)

73. (canceled)

74. (canceled)

75. (canceled)

76. (canceled)

77. (canceled)

78. (canceled)

79. (canceled)

80. (canceled)

81. The method of claim 1, wherein the dose of HIF-PH inhibitor is at least 2 mg/kg PO per day.

82. (canceled)

83. (canceled)

84. (canceled)

85. (canceled)

86. (canceled)

87. (canceled)

88. (canceled)

89. (canceled)

90. (canceled)

91. (canceled)

92. The method of claim 81, wherein the dose is 1 to 30 mg.

93. (canceled)

94. (canceled)

95. (canceled)

96. (canceled)

97. (canceled)

98. (canceled)

99. The method of claim 1, wherein the HIF-PH inhibitor is administered orally.

100. The method of claim 81, wherein the dose is administered daily.

101. The method of claim 81, wherein the dose is administered as a plurality of equally or unequally divided sub-doses.
